SEQUENCE LISTING (18 sequences)

SEQ ID NO: 1 - DNA, 10005 bp, synthetic construct
  source 1..10005: mol_type = other DNA
  misc_feature 1: note = Complete Sequence and Map of PNI V101 Cloning Vector
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacgccaccaagtctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 2 - DNA, 3822 bp, Severe acute respiratory syndrome coronavirus 2
  source 1..3822: mol_type = genomic DNA; note = Severe acute respiratory syndrome coronavirus 2 isolate Wuhan-Hu-1
  misc_feature 1: note = Original nucleotide sequence encoding SARS-CoV-2 Spike protein
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 3822 bp, synthetic construct
  source 1..3822: mol_type = other DNA
  misc_feature 1: note = PNI A1 Codon optimized nucleotide sequence encoding SARS-CoV-2 Spike protein
atgttcgtgttcctggtgctgctgcccctggtgtcctcacagtgcgtgaatctgaccactcgtactcagctgcccccagcatacaccaattctttcaccagaggggtctactaccccgacaaagtgttcaggagttccgtcttgcattccacacaggacctgttcctgccattcttcagcaatgtcacctggttccacgcgatccacgtttctggcaccaacgggacaaagcggttcgacaaccctgttctgccgttcaacgatggcgtgtactttgcgtccaccgagaaaagcaacatcatccgcggctggatcttcgggactaccctggactcgaagactcaatcgctgctgatcgtcaacaacgcaacgaacgtggtgatcaaggtgtgcgaattccagttctgcaacgacccgttccttggcgtctactaccacaagaacaacaagagttggatggagtccgagttccgcgtttactcttccgccaacaactgcacattcgagtacgtgtcgcaacctttcctgatggacctggagggcaagcagggcaacaaaaagaacctgcgcgagttcgtcttcaagaacattgtcggctacttcaagatttactccaagcacaccccgatcaaccttgtgcgcgatctacctcagggattctctgctctggagccactggtggacctgcccattgggatcaatataacgcgtttccagaccctgctggctttacaccgctcctatttgacccccgccgattcttcctccggctggacggccggcgccgcagcctattacgtaggctacctgcagccgcgcaccttccttttaaagtacaacgagaacggcactatcaccgacgcagtagattgtgctctggaccctctgagcgagaccaaatgcactctgaagtcttttacagtggagaagggaatctatcagggtgccaacttccgcgtgcagcccaccgagagcatcgacaggttccccaacatcacgaatctgtgcccattcggcgaggtgttcaacgccacccgcttcgcatccgtgtacgcgtggaaccggaagcgcatctccaattgcgtggcggactactctgttctcgtaaactccgcttcgttctccacgttcaagtgctatggcgtctcccctaccaaactaaacgacctgtgctttaccaacgtatacgcggactcgtttgtgattcgcggcgtcgaggtgcgccagatcgctccgggccagactggcaagattgcagactacaactacaagctgcccgacgacttcaccggctgcgtgatcgcgtgggttggaaacaacctggacagcaaggtgggggggaacgtaaactacctgtaccgtctgttccgcaagatgaacctgaggccgggagagcgcgatatttctaccgagatttaccaggccggctccaccccttgcaacggcgtggagggcttcaactgctacttccccctgcagagctacggcttccagccgacgatcggtgtcggctatcagccataccgggtggtggtgttgagtttcgagctcctgcacgcccccgccactgtctgtggacccaagaagtccaccaacctttacaagaacaagtgcgtgaacttcaacttcaacgggctgaccggcacgggtgtgctgaccgaaagcaacaagaaatttctccccttccagcagtttggacgagacatcgcggacacgaccgacgctgtgagggaccctcagaccctggagatcctggacatcactccttgctcctttggtggcgtgtccgtgatcacgcccggtaccaaccgaagcaaccaggtcgccgtgctgtaccaagacgtgaactgcaccgaggtgcccgtggcgatccacgccgaccagttgaccccaacctggcgagtgtactcgaccgggtccaacgtgttccagactcgcgccggctgcttgatcggtgccgagcacgtcaacaacagctacgagtgcgacatccccatcggtgcgggtatttgtgcctcctaccagacccagaccaactccccgcggcgtgcgcgcagtgtggcttcccagagcatcatcgcctacaccatgtctctgggtgcggagaattctgtcgccacgagcaacaattcgatcgctatccccaccaacttcaccatcagcgtcaccaccgaaatcctccccgtgtccatgaccaagacctcggtcgattgcactatgtatatttgcggggactcaaccgaatgctccaaccttctgctccagtacggctctttttgtacgcaactcaaccgagctttgactgggatcgcggtcgagcaggacaaaaatacccaggaggtgttcgcgcaggtgaagcagatatacaagaccccccccatcaaggatttcggcggcttcaacttctcacagatcctacccgacccgtccaaaccctcgaagcgcagcttcatcgaggacctgctgttcaacaaagtgacactggccgacgctggatttatcaagcaatacggcgattgcctgggcgacatcgcggcccgcgacctgatctgcgcccagaaattcaacgggctaaccgtgcttcccccgctgctgaccgacgagatgatcgcccagtacacgtcggcccttctggctggcacgatcaccagcggctggaccttcggcgctggcgccgcgctccagatcccctttgccatgcagatggcctacagattcaacggcatcggcgtgacacagaatgtgctgtacgagaaccagaagttgatcgccaatcagttcaacagcgcgataggcaagatccaggattcattgtcatccaccgcaagcgccctgggcaagctgcaggacgtggtcaaccagaacgctcaggctctcaacacactggtgcctcagttgtccagcaactttggtgccatctcctccccactcaacgacatcctttcgcgtctggacaaagtcgaggccgaggtgcagatcgacaggctgatcaccggacgcttgcagagcttgcagacttacgtgacccaacagctgatacgcgctgcagaaatccgggcgtccgccaacctggcagcgaccaagatgtccgagtgcgtgctgggacagtccaagcgagacgacttctgtggaaaaggctaccacctgatgtcctttcctcagtccgccccccatggagtggtgtttctgcacgtcacctacgtgcccgcccaagagaagaacttcaccactgcgcctgcgatctgtcacgacgggaaggctcactttccgcgcgagggggtgttcgtctccaacggcacccactggttcgtgactcagcgcaacttttacgagccccagatcatcaccaccgacaacactttcgtatccggcaattgcgatgtggtaattggtattgtgaacaacaccgtgtacgaccccttgcagccagaactcgactctttcaaggaggagctcgacaagtacttcaagaaccatacatcgccagatgtggacttaggcgacatctcaggaatcaacgcctccgtggtcaacatccagaaggagatcgaccgcctgaacgaggtcgccaagaacctaaacgaaagcctgatcgacctgcaggagctgggcaagtacgagcagtacatcaaatggccgtggtacatctggcttggtttcatcgctggcctcattgccattgtgatggtgaccatcatgctatgctgtatgacctcctgctgttcttgcctgaagggctgttgttcttgcggctcctgttgcaaattcgacgaaggcgcttcagagccggtgttgaagggcgtcaagctgcactatacctga

SEQ ID NO: 4 - DNA, 3822 bp, synthetic construct
  source 1..3822: mol_type = other DNA
  misc_feature 1: note = PNI A2
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagcccgagttcagagtgtacagcagcgccaacaactgcaccttcgagtacgtgagccagcccttcctgatggacctggagggcaagcagggcaacaagaagaacctgagagagttcgtgttcaagaacatcgtgggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccgccgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagggcgccaacttcagagtgcagcccaccgagagcatcgacagattccccaacatcaccaacctgtgccccttcggcgaggtgttcaacgccaccagattcgccagcgtgtacgcctggaacagaaagagaatcagcaactgcgtggccgactacagcgtgctggtgaacagcgccagcttcagcaccttcaagtgctacggcgtgagccccaccaagctgaacgacctgtgcttcaccaacgtgtacgccgacagcttcgtgatcagaggcgtggaggtgagacagatcgcccccggccagaccggcaagatcgccgactacaactacaagctgcccgacgacttcaccggctgcgtgatcgcctgggtgggcaacaacctggacagcaaggtgggcggcaacgtgaactacctgtacagactgttcagaaagatgaacctgagacccggcgagagagacatcagcaccgagatctaccaggccggcagcaccccctgcaacggcgtggagggcttcaactgctacttccccctgcagagctacggcttccagcccaccatcggcgtgggctaccagccctacagagtggtggtgctgagcttcgagctgctgcacgcccccgccaccgtgtgcggccccaagaagagcaccaacctgtacaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacagaagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggccaccagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgccccagctgagcagcaacttcggcgccatcagcagccccctgaacgacatcctgagcagactggacccccccgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagacgacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgagggcgccagcgagcccgtgctgaagggcgtgaagctgcactacacctga

SEQ ID NO: 5 - DNA, 3822 bp, synthetic construct
  source 1..3822: mol_type = other DNA
  misc_feature 1: note = PNI A3
atgttcgtgttcctggtgctgctgcccctggtgtcctcacagtgcgtgaatctgaccactcgtactcagctgcccccagcatacaccaattctttcaccagaggggtctactaccccgacaaagtgttcaggagttccgtcttgcattccacacaggacctgttcctgccattcttcagcaatgtcacctggttccacgcgatccacgtttctggcaccaacgggacaaagcggttcgacaaccctgttctgccgttcaacgatggcgtgtactttgcgtccaccgagaaaagcaacatcatccgcggctggatcttcgggactaccctggactcgaagactcaatcgctgctgatcgtcaacaacgcaacgaacgtggtgatcaaggtgtgcgaattccagttctgcaacgacccgttccttggcgtctactaccacaagaacaacaagagttggatggagcccgagttccgcgtttactcttccgccaacaactgcacattcgagtacgtgtcgcaacctttcctgatggacctggagggcaagcagggcaacaaaaagaacctgcgcgagttcgtcttcaagaacattgtcggctacttcaagatttactccaagcacaccccgatcaaccttgtgcgcgatctacctcagggattctctgctctggagccactggtggacctgcccattgggatcaatataacgcgtttccagaccctgctggctttacaccgctcctatttgacccccgccgattcttcctccggctggacggccggcgccgcagcctattacgtaggctacctgcagccgcgcaccttccttttaaagtacaacgagaacggcactatcaccgacgcagtagattgtgctctggaccctctgagcgagaccaaatgcactctgaagtcttttacagtggagaagggaatctatcagggtgccaacttccgcgtgcagcccaccgagagcatcgacaggttccccaacatcacgaatctgtgcccattcggcgaggtgttcaacgccacccgcttcgcatccgtgtacgcgtggaaccggaagcgcatctccaattgcgtggcggactactctgttctcgtaaactccgcttcgttctccacgttcaagtgctatggcgtctcccctaccaaactaaacgacctgtgctttaccaacgtatacgcggactcgtttgtgattcgcggcgtcgaggtgcgccagatcgctccgggccagactggcaagattgcagactacaactacaagctgcccgacgacttcaccggctgcgtgatcgcgtgggttggaaacaacctggacagcaaggtgggggggaacgtaaactacctgtaccgtctgttccgcaagatgaacctgaggccgggagagcgcgatatttctaccgagatttaccaggccggctccaccccttgcaacggcgtggagggcttcaactgctacttccccctgcagagctacggcttccagccgacgatcggtgtcggctatcagccataccgggtggtggtgttgagtttcgagctcctgcacgcccccgccactgtctgtggacccaagaagtccaccaacctttacaagaacaagtgcgtgaacttcaacttcaacgggctgaccggcacgggtgtgctgaccgaaagcaacaagaaatttctccccttccagcagtttggacgagacatcgcggacacgaccgacgctgtgagggaccctcagaccctggagatcctggacatcactccttgctcctttggtggcgtgtccgtgatcacgcccggtaccaaccgaagcaaccaggtcgccgtgctgtaccaagacgtgaactgcaccgaggtgcccgtggcgatccacgccgaccagttgaccccaacctggcgagtgtactcgaccgggtccaacgtgttccagactcgcgccggctgcttgatcggtgccgagcacgtcaacaacagctacgagtgcgacatccccatcggtgcgggtatttgtgcctcctaccagacccagaccaactccccgcggcgtgcgcgcagtgtggcttcccagagcatcatcgcctacaccatgtctctgggtgcggagaattctgtcgccacgagcaacaattcgatcgctatccccaccaacttcaccatcagcgtcaccaccgaaatcctccccgtgtccatgaccaagacctcggtcgattgcactatgtatatttgcggggactcaaccgaatgctccaaccttctgctccagtacggctctttttgtacgcaactcaaccgagctttgactgggatcgcggtcgagcaggacaaaaatacccaggaggtgttcgcgcaggtgaagcagatatacaagaccccccccatcaaggatttcggcggcttcaacttctcacagatcctacccgacccgtccaaaccctcgaagcgcagcttcatcgaggacctgctgttcaacaaagtgacactggccgacgctggatttatcaagcaatacggcgattgcctgggcgacatcgcggcccgcgacctgatctgcgcccagaaattcaacgggctaaccgtgcttcccccgctgctgaccgacgagatgatcgcccagtacacgtcggcccttctggctggcacgatcaccagcggctggaccttcggcgctggcgccgcgctccagatcccctttgccatgcagatggcctacagattcaacggcatcggcgtgacacagaatgtgctgtacgagaaccagaagttgatcgccaatcagttcaacagcgcgataggcaagatccaggattcattgtcatccaccgcaagcgccctgggcaagctgcaggacgtggtcaaccagaacgctcaggctctcaacacactggtgcctcagttgtccagcaactttggtgccatctcctccccactcaacgacatcctttcgcgtctggacccccccgaggccgaggtgcagatcgacaggctgatcaccggacgcttgcagagcttgcagacttacgtgacccaacagctgatacgcgctgcagaaatccgggcgtccgccaacctggcagcgaccaagatgtccgagtgcgtgctgggacagtccaagcgagacgacttctgtggaaaaggctaccacctgatgtcctttcctcagtccgccccccatggagtggtgtttctgcacgtcacctacgtgcccgcccaagagaagaacttcaccactgcgcctgcgatctgtcacgacgggaaggctcactttccgcgcgagggggtgttcgtctccaacggcacccactggttcgtgactcagcgcaacttttacgagccccagatcatcaccaccgacaacactttcgtatccggcaattgcgatgtggtaattggtattgtgaacaacaccgtgtacgaccccttgcagccagaactcgactctttcaaggaggagctcgacaagtacttcaagaaccatacatcgccagatgtggacttaggcgacatctcaggaatcaacgcctccgtggtcaacatccagaaggagatcgaccgcctgaacgaggtcgccaagaacctaaacgaaagcctgatcgacctgcaggagctgggcaagtacgagcagtacatcaaatggccgtggtacatctggcttggtttcatcgctggcctcattgccattgtgatggtgaccatcatgctatgctgtatgacctcctgctgttcttgcctgaagggctgttgttcttgcggctcctgttgcaaattcgacgaaggcgcttcagagccggtgttgaagggcgtcaagctgcactatacctga

SEQ ID NO: 6 - DNA, 3822 bp, synthetic construct
  source 1..3822: mol_type = other DNA
  misc_feature 1: note = PNI A4
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 7 - DNA, 3822 bp, synthetic construct
  source 1..3822: mol_type = other DNA
  misc_feature 1: note = PNI A5
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaaaagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaaaagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaaaaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccaggagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 8 - DNA, 69 bp, synthetic construct
  source 1..69: mol_type = other DNA
  misc_feature 1: note = tPA signal sequence
atggacgccatgaagcggggcctctgctgtgttctgctgctctgcggcgccgtgttcgtgagtaactcg

SEQ ID NO: 9 - DNA, 798 bp, synthetic construct
  source 1..798: mol_type = other DNA
  misc_feature 1: note = P2A sfGFP sequence
atgggaagcggaatgggaagcggagccacgaacttctctctgttaaagcaagcaggagatgttgaagaaaaccccgggcctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 10 - DNA, 138 bp, synthetic construct
  source 1..138: mol_type = other DNA
  misc_feature 1: note = Linker-Fibritin foldon sequence
tctggtggcggaggctcgggcggaggtgggtcgggtggcggcggatcaggttatattcctgaagctccaagagatgggcaagcttacgttcgtaaagatggcgaatgggtattcctttctacctttttatcaccagca

SEQ ID NO: 11 - DNA, 101 bp, synthetic construct
  source 1..101: mol_type = other DNA
  misc_feature 1: note = Signal sequence (SS)
atgtttgtttttcttgttttattgccactagtctctagtcagtgtgttaatcttacaaccagaactcaattaccccctgcatacactaattctttcacacg

SEQ ID NO: 12 - DNA, 136 bp, synthetic construct
  source 1..136: mol_type = other DNA
  misc_feature 1: note = 3' UTR AES
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 13 - DNA, 142 bp, synthetic construct
  source 1..142: mol_type = other DNA
  misc_feature 1: note = 3' UTR mtRNR1
caagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 14 - DNA, 29903 bp, Severe acute respiratory syndrome coronavirus 2
  source 1..29903: mol_type = genomic DNA; note = Severe acute respiratory syndrome coronavirus 2 isolate Wuhan-Hu-1
  misc_feature 1: note = NCBI Reference Sequence: NC_045512.2 (complete genome)
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 15 - DNA, 11 bp, synthetic construct
  source 1..11: mol_type = other DNA
aaaagaagagc

SEQ ID NO: 16 - DNA, 11 bp, synthetic construct
  source 1..11: mol_type = other DNA
ttttcttctcg

SEQ ID NO: 17 - DNA, 11 bp, synthetic construct
  source 1..11: mol_type = other DNA
gctcttctttt

SEQ ID NO: 18 - DNA, 11 bp, synthetic construct
  source 1..11: mol_type = other DNA
cgagaagaaaa

(19) United States
(12) Patent Application Publication
(10) Pub. No.: US 2025/0228926 A1
(43) Pub. Date: Jul. 17, 2025

(54) RNA VACCINE LIPID NANOPARTICLES

(71) Applicant: GLOBAL LIFE SCIENCES SOLUTIONS CANADA ULC, Vancouver, British Columbia (CA)

(72) Inventors: Andy John Geall, Carlsbad, CA (US); Suraj Abraham, Laval, Quebec (CA); Anitha Thomas, New Westminster, British Columbia (CA); Sitalakshmi Radhakrishnan Thampatty, Vancouver (CA); Sijo Chemmannur, Vancouver (CA)

(21) Appl. No.: 18/690,988

(22) PCT Filed: Oct. 7, 2022
(86) PCT No.: PCT/IB2022/059616

§ 371 Date: Mar. 11, 2024

Related U.S. Application Data
(60) Provisional application No. 63/253,489, filed on Oct. 7, 2021.

(51) Int. Cl. A61K 39/215; A61K 9/1272; A61K 9/51; A61K 39/00; A61P 31/14; B82Y 5/00; C07K 14/005; C12N 15/86

(52) U.S. Cl. CPC A61K 39/215; A61K 9/1272; A61K 9/5123; A61P 31/14; C07K 14/005; C12N 15/86; A61K 2039/53; A61K 2039/55555; B82Y 5/00; C12N 2770/20022; C12N 2770/20034; C12N 2770/36122; C12N 2770/36134; C12N 2770/36143; C12N 2830/50

(57) ABSTRACT

Disclosed are recombinant expression vectors useful as RNA vaccines. Also disclosed are pharmaceutically acceptable carriers for the recombinant expression vectors, particularly lipid nanoparticles.

### CROSS REFERENCE TO RELATED APPLICATION

This patent application claims the benefit of U.S. Provisional Patent Application No. 63/253,489, filed Oct. 7, 2021, which is incorporated by reference in its entirety herein.

Incorporated by reference in its entirety herein is a computer-readable nucleotide/amino acid sequence listing submitted concurrently herewith and identified as follows: One 86,367 Byte XML file named “764862_ST26.XML,” dated Oct. 6, 2022.

### FIELD OF THE INVENTION

The field of the invention relates to methods and compositions for RNA vaccines, particularly those formulated in lipid nanoparticles.

### BACKGROUND OF THE INVENTION

RNA vaccines may be useful for the treatment or prevention of conditions caused by any of a variety of pathogens. Despite advancements in the field, challenges to RNA vaccine development may include providing and maintaining the stability of the pharmaceutically acceptable carrier, versatility of the recombinant expression vector, and practical manufacturing methods. Attempts to successfully lyophilize RNA vaccine compositions may present further challenges. Accordingly, there is a need for improved RNA vaccine compositions and methods of manufacturing them.

### BRIEF SUMMARY OF THE INVENTION

An aspect of the invention provides a recombinant expression vector comprising a nucleotide sequence comprising: (a) a Venezuelan Equine Encephalitis Virus (VEEV) 5′ untranslated region (5′-UTR); (b) a nucleotide sequence encoding VEEV non-structural proteins nsP1, nsP2, nsP3 and nsP4; (c) a VEEV 26S sub-genomic promoter; (d) an engineered multiple cloning site (MCS); (e) a VEEV 3′ untranslated region (3′-UTR); and (f) a nucleotide sequence encoding a VEEV poly A sequence.

Another aspect of the invention provides a pharmaceutical composition comprising the inventive recombinant expression vector and a pharmaceutically acceptable carrier.

### BRIEF DESCRIPTION OF THE SEVERAL VIEWS OF THE DRAWINGS

FIG. 1 is a map of a synthetic cloning vector (PNI V101) for self-amplifying mRNA according to an aspect of the invention. The vector includes self-replicating machinery along with Poly (A) tail with engineered multiple cloning site (MCS) incorporated within the cloning vector to allow synthesis of large size saRNA that can generate naked positive strand alphavirus RNA replicons containing the gene of interest.

FIG. 2 is a schematic illustrating the linearization of the PNI V101 vector with an insertion of a 3822 bp gene sequence encoding for SARS-COV-2 spike protein. PmeI linearization (top of figure) generates an 11-nucleotide blunt end overhang after the poly (A) tail. BspQI linearization right at the end of the poly (A) tail produces a staggered end of 3 thymine nucleotides.

FIG. 3A is a photograph of a gel illustrating in vitro expression levels for nCOV antigen encoded self-amplifying mRNAs (saRNAs) derived from PNI V101. The gel shows differences in SARS-COV-2 spike protein expression in the first and second generation self-amplifying mRNA (saRNA) transfected human embryonic kidney cells. Beta actin, no transfection, and a positive control commercial spike protein sequence nCov CleanCap AU in PNI516 VACCMIX-B LNP served as control.

FIG. 3B is a photograph of gels illustrating in vitro expression levels for nCOV antigen encoded saRNA and controls, as well levels of protein expression for VEEV nsp2.

FIG. 4 shows graphs showing size and PDI results (left graph) and encapsulation efficiency (right graph) of saRNAs synthesized from 1^{st} and 2^{nd} generation A1 and A3 as well as A4 and A5 type vaccine vector LNP.

FIG. 5 is a graph showing anti-SARS-COV-2 spike protein specific IgG levels following treatment with saRNAs encoding different nCOV spike protein antigen designs in PNI V101 Vector.

FIG. 6 is a graph showing anti-SARS-COV-2 spike protein specific IgG levels in vivo following treatment with saRNAs encoding different nCOV spike protein antigen designs in PNI V101 Vector.

FIGS. 7A-7B are graphs showing serum anti-SARS-COV-2 Spike Protein IgG Titer following treatment with saRNAs encoding different nCOV spike protein antigen designs in PNI V101 Vector.

FIG. 8 is a graph showing the results of the Pseudovirion Neutralization Assay (PNA).

FIG. 9 is a graph showing serum anti SARS-COV-2 spike protein specific IgG level data following treatment with saRNAs encoding different nCOV spike protein antigen designs in PNI V101 Vector.

FIG. 10 is a graph showing the frequency (percentage) of IFN gamma+, TNF alpha, and IL2+ cell with respect to CD4+ve T cell response against SARS-COV-2, Wuhan Strain virus.

FIG. 11 is a graph showing the CD8+ve T cell response against vaccine vector LNP, A4 and A5 type versus PBS or naked saRNA controls over a variety of markers (IL2+; IFN gamma+, and TNF alpha+).

FIG. 12 is a graph showing serum anti SARS-COV-2 spike protein specific IgG levels for NP6 and NP8 and various antigenic vector vaccines.

FIG. 13A is a graph showing the size and PDI of vaccine vector LNP for different ionizable lipids.

FIG. 13B is a graph showing the encapsulation efficiency for the samples described for FIG. 13A.

FIG. 14 is a graph showing SARS Cov-2 Spke protein specific IgG levels in sera of mice which had received one of eleven different ionizable lipid vaccine vector LNP at NP6 and NP8. The specific antigenic component was A3.

FIG. 15 are images showing the results of fluorescence microscopy study of HEK293 cells treated with positive control, negative control, eGFP PNI V101 saRNA, and eGFP B18R PNI V101 24 hours post electroporation.

FIG. 16 are images showing the results of fluorescence microscopy study of HEK293 cells treated with positive control, negative control, eGFP PNI V101 saRNA at 1 ug and 4 μg, at 24 hours post electroporation.

FIG. 17 are images showing the results of a bright field and green filter fluorescence microscopy study of Lipofectamine 2000 control treated BHK cells (top row) and eGFP saRNA.

FIG. 18 is a graph showing the results of a study of cell populations at various MFI levels for eGFP PNI V101 at 4 μg, at 1 μg, negative control at both concentrations, GFP positive control, and mock transfection.

FIG. 19 is a photograph of a gel illustrating in vitro expression levels for H1N1 HA antigen encoded saRNAs derived from PNI-v101.

FIG. 20A is a graph showing plaque reduction neutralization test at serum dilution 50 for the alpha variant of the SARS NCov-2 virus.

FIG. 20B is a graph showing plaque reduction neutralization test at serum dilution 90 for the alpha variant of the SARS NCov-2 virus.

FIG. 20C is a graph showing plaque reduction neutralization test at serum dilution 50 for the Beta variant of the SARS NCov-2 virus.

FIG. 20D is a graph showing plaque reduction neutralization test at serum dilution 50 for the delta variant of the SARS NCov-2 virus.

FIG. 20E is a graph showing plaque reduction neutralization test at serum dilution 90 for the delta variant of the SARS NCov-2 virus.

FIG. 21A is a graph showing the particle size of LNPs with various ionizable lipids after 10 weeks of storage about −80° C.

FIG. 21B is a graph showing the encapsulation efficiency of various LNPs with various ionizable lipids after 10 weeks of storage about −80° C.

FIG. 22 is an image of an electrophoresis gel showing the saRNA integrity of various LNP formulations after 10 weeks of storage at −80 Deg. C.

FIG. 23 is a graph showing SARS-COV-2 spike protein concentration after 0.25 μg/mL treatment of HEK 293 cells with various LNP formulations.

FIG. 24 is a graph showing the size and PDI of the vaccine vector LNPs of Table 34.

FIG. 25 is a graph showing the encapsulation efficiency of the vaccine vector LNPs of Table 34.

FIG. 26 is a graph showing the stability of the sizes and PDI of LNPs prepared with various ionizable lipids and structural lipids following no storage, after a 1 week storage, and after a 1 month storage.

FIG. 27 is a graph showing the stability of the encapsulation efficiency of LNPs prepared with various ionizable lipids and structural lipids following no storage, after a 1 week storage, and after a 1 month storage.

FIGS. 28-29 are photographs of gels illustrating A5 antigen extracted from LNPs prepared with various ionizable lipids (PNI 516, PNI 585, and PNI 560) and various structural lipids following 1 week of storage (28) and following 1 month of storage (29).

FIG. 30 shows photographs of gels illustrating A5 antigen extracted from LNPs prepared with various ionizable lipids (PNI 542, PNI 580, PNI 563, and PNI 586) and various structural lipids following 1 week of storage (left) and following 1 month of storage (right).

FIG. 31 is a graph showing the neutralizing antibodies against SARS-COV-2 in mice sera. Symbols and horizontal lines represent individual titers of each sample and mean titers of each group, respectively. Serum titers were calculated based on PRNT90 and expressed as reciprocals Log2 dilution.

FIG. 32 is a graph showing the impact of native and codon-optimized antigenic gene sequences on SARS-COV-2 Spike protein IgG levels in serum obtained from animals treated with different antigen encoded saRNA encapsulated in PNI 516 LNPs.

FIG. 33 is a graph showing the efficiency of neutralizing the SARS-COV-2 Delta variant strain in the serum obtained from animals treated with different antigen encoded saRNA PNI 516 LNPs.

FIG. 34 is a graph showing the efficiency of neutralizing the SARS-COV-2 Omicron variant strain in the serum obtained from animals treated with different antigen encoded saRNA PNI 516 LNPs.

FIG. 35 is a graph showing the levels of SARS-COV-2 specific IgG titer in non-human Primates (NHPs) treated with PNI A5 antigen encapsulated in lipid nanoparticles from two different ionizable lipid compositions.

FIG. 36 is a graph showing the efficiency of neutralizing the SARS-COV-2 Delta variant strain in the serum obtained from animals treated with PNI A5 antigen encapsulated in lipid nanoparticles from different types of ionizable lipid compositions.

FIG. 37 is a graph showing the efficiency of neutralizing the SARS-COV-2 Omicron variant strain in the serum obtained from animals treated with PNI A5 antigen encapsulated in lipid nanoparticles from different types of ionizable lipid compositions.

FIG. 38 is a graph showing the influenza A/California/07/2009 (H1N1) Hemagglutinin Inhibition (HAI) titer levels following treatment with H1N1 HA saRNA encapsulated in PNI 516 LNPs.

FIG. 39 is a graph showing the hydrodynamic particle size of influenza A/California/07/2009 (H1N1) HA PNI 516 LNP samples.

FIG. 40 is a graph show the Zeta potential of influenza A/California/07/2009 (H1N1) HA PNI 516 LNP samples.

FIG. 41 is a graph showing the encapsulation efficiency (% EE) of influenza A/California/07/2009 (H1N1) HA PNI 516 LNP samples.

### DETAILED DESCRIPTION OF THE INVENTION

The inventive compositions may provide any one or more of a variety of advantages. Because of the short production times, RNA vaccines may be ideal for responding quickly to new threats like the COVID-2019 virus. Furthermore, nucleic-acid-based vaccines may offer advantages over traditional vaccines in terms of safety and efficacy. Messenger RNA (“mRNA”) vaccines may compare favorably against vaccines based on DNA, which need to cross the nuclear membrane in order to work and carry the risk of integration into the host genome. Self-amplifying mRNA vaccines can be effective at even lower doses than mRNA vaccines, because each self-amplifying mRNA vaccine manufactures multiple mRNA units. Self-amplifying mRNA may provide the advantage of prolonged translation and high yield of target antigen compared to other mRNA vaccines.

An aspect of the invention provides a recombinant expression vector useful for encoding any desired mRNA target antigen for a vaccine. In an aspect of the invention, the recombinant expression vector is RNA. In some aspects, the vector is an RNA that encodes a target antigen. This target antigen elicits an immune response which recognizes the target antigen, to provide immunity against the target antigen.

The recombinant expression vector may comprise a nucleotide sequence comprising: (a) a Venezuelan Equine Encephalitis Virus (VEEV) 5′ untranslated region (5′-UTR); (b) a nucleotide sequence encoding VEEV non-structural proteins nsP1, nsP2, nsP3 and nsP4; (c) a VEEV 26S sub-genomic promoter; (d) an engineered multiple cloning site (MCS); (e) a VEEV 3′ untranslated region (3′-UTR); and (f) a nucleotide sequence encoding a VEEV poly A sequence.

In an aspect of the invention, the nucleotide sequence encoding the VEEV poly A sequence comprises from 38 to 40 base pairs, from 38 to 39 base pairs, from 39 to 40 base pairs, or 38, 39, or 40 base pairs. This Poly A base pair length is useful for in vitro cell-free synthesis of self-amplifying mRNA (saRNAs) encoding various genes of interest with a desired Poly (A) tail length. It also facilitates the stability of the replicon RNA that provides for the negative strand synthesis of the replicon RNA encoding various genes of interest within any in vivo mammalian cell system.

In an aspect of the invention, the VEEV 26S sub-genomic promoter is a VEEV TC83 strain 26S sub-genomic promoter.

In an aspect of the invention, a gene of interest (GOI) is insertable at the MCS. The GOI is not limited. The GOI may be any reporter or therapeutic target gene of interest either in a single gene element or a multiple gene element cassette format. The GOI may be a genetic element or elements intended for expression to achieve a therapeutic goal, e.g., immunization. In an aspect of the invention, the GOI encodes a target antigen for a vaccine. After administration of the vector, the RNA is translated in vivo into the target antigen polypeptide. The target antigen polypeptide can elicit an immune response in the recipient.

The target antigen may elicit an immune response against a pathogen (e.g. a bacterium, a virus, a fungus or a parasite) but, in some aspects, it elicits an immune response against an allergen or a tumor antigen. The immune response may comprise an antibody response (usually including IgG) and/or a cell mediated immune response. The target antigen polypeptide will typically elicit an immune response which recognizes the corresponding pathogen (or allergen or tumor) polypeptide, but in some aspects the polypeptide may act as a mimotope to elicit an immune response which recognizes a saccharide. The target antigen will typically be a surface polypeptide e.g., an adhesin, a hemagglutinin, an envelope glycoprotein, or a spike glycoprotein, etc. The RNA molecule can encode a single polypeptide target antigen or multiple polypeptides. Multiple target antigens can be presented as a single target antigen polypeptide (fusion polypeptide) or as separate polypeptides. If target antigens are expressed as separate polypeptides from a replicon, then one or more of these may be provided with an upstream IRES or an additional viral promoter element. Alternatively, multiple target antigens may be expressed from a polyprotein that encodes individual target antigens fused to a short autocatalytic protease (e.g., foot-and-mouth disease virus 2A protein), or as inteins. In certain aspects, target antigen polypeptides (e.g., 1, 2, 3, 4, 5, 6, 7, 8, 9, 10, or more target antigens) may be used, either alone or together with a RNA molecule, such as a self-replicating RNA, encoding one or more target antigens (either the same or different as the polypeptide target antigens).

In some aspects, the target antigen elicits an immune response against Coronavirus. Coronavirus target antigens include, but are not limited to, those derived from a SARS COV-1 and SARS-COV-2. In an aspect, the target antigen may be a full-length, pre-fusion form of the SARS-COV-2 spike protein. The GOI may be a nucleotide sequence encoding a SARS-COV-2 amino acid sequence, for example, the nucleotide sequence of any one of SEQ ID NOs: 2-7. In this regard, the recombinant expression vector may further comprise the nucleotide sequence of any one of SEQ ID NO: 2-7 inserted at the engineered MCS. In an aspect, the GOI may be a codon-optimized nucleotide sequence, for example, the nucleotide sequence of any one of SEQ ID NOs: 3-7. Codon-optimized SARS-COV-2 nucleotide sequence may provide any one or more of increased antigenic expression in mammalian cell systems, increased neutralizing antibody production, and improved long-term T-cell response against SARS-COV-2 infection.

In an aspect of the invention, the recombinant expression vector further comprises a bicistronic gene element inserted at the engineered MCS, wherein the bicistronic gene element comprises (i) the nucleotide sequence of any one of SEQ ID NO: 2-7 and (ii) the nucleotide sequence of SEQ ID NO: 9. Such a bicistronic gene element may provide for detection of SARS-COV-2 spike protein expression in mammalian cell systems using fluorescent based detection techniques.

In an aspect of the invention, the recombinant expression vector further comprises a bicistronic gene element inserted at the engineered MCS, wherein the bicistronic gene element comprises (i) the nucleotide sequence encoding a SARS-COV-2 spike protein amino acid sequence or a modified SARS-COV-2 spike protein amino acid sequence and (ii) a nucleotide sequence encoding a leader sequence. The nucleotide sequence encoding the modified SARS-COV-2 spike protein may be the same as that of the reference sequence NC_045512.2 (SEQ ID NO: 14) with the exception that the nucleotide sequence encoding the modified SARS-COV-2 spike protein incorporates one or more mutations relative to the reference sequence NC_045512.2 (SEQ ID NO: 14). The SARS-COV-2 spike protein may be a SARS-COV-2 spike protein from any strain of the SARS-COV-2 virus. The nucleotide sequence encoding the leader sequence may comprise the nucleotide sequence of SEQ ID NO: 8 or 11 or a sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleotide sequence of SEQ ID NO: 8 or 11. Such nucleotide sequences may potentiate enhanced antigenic expression and/or immune response in the translated antigenic protein.

In an aspect of the invention, the recombinant expression vector further comprises a bicistronic gene element inserted at the engineered MCS, wherein the bicistronic gene element comprises (i) the nucleotide sequence encoding a SARS-COV-2 spike protein amino acid sequence or a modified SARS-COV-2 spike protein amino acid sequence and (ii) a 3′ untranslated region (UTR). The nucleotide sequence encoding the modified SARS-CoV-2 spike protein may be as described herein with respect to other aspects of the invention. In an aspect, the 3′ UTR comprises the nucleotide sequence of SEQ ID NO: 12 or SEQ ID NO: 13. Such nucleotide sequences may potentiate enhanced post-transcriptional RNA stability and/or proper translation of the antigenic protein.

In some aspects, the target antigen elicits an immune response against human influenza virus. In some aspects, the target antigen elicits an immune response against Neisseria meningitides. Neisseria meningitides target antigens include, but are not limited to, membrane proteins such as adhesins, autotransporters, toxins, iron acquisition proteins, and factor H binding protein. A combination of three useful Neisseria meningitides polypeptides is disclosed in Giuliani et al., PNAS, 103 (29): 10834-9 (2006). In some aspects, the target antigen elicits an immune response against Streptococcus pneumonia. Streptococcus pneumonia target antigens include those disclosed in WO2009/016515, the RrgB pilus subunit, the beta-N-acetyl-hexosaminidase precursor (spr0057), spr0096, general stress protein GSP-781 (spr2021, SP2216), serine/threonine kinase StkP (SP1732), and pneumococcal surface adhesin PsaA.

In some aspects, the target antigen elicits an immune response against hepatitis viruses. Hepatitis virus target antigens can include hepatitis B virus surface antigen (HBsAg), hepatitis C virus, delta hepatitis virus, hepatitis E virus, or hepatitis G virus antigens. In some aspects, the target antigen elicits an immune response against Rhabdovirus. Rhabdovirus target antigens include, but are not limited to, those derived from a Rhabdovirus, such as a Lyssavirus (e.g. a Rabies virus) and Vesiculovirus (VSV). In some aspects, the target antigen elicits an immune response against Caliciviridae. Caliciviridae target antigens include, but are not limited to, those derived from Calciviridae, such as Norwalk virus (Norovirus), and Norwalk-like Viruses, such as Hawaii Virus and Snow Mountain Virus.

In some aspects, the target antigen elicits an immune response against avian infectious bronchitis (IBV), Mouse hepatitis virus (MHV), and Porcine transmissible gastroenteritis virus (TGEV). In some aspects, the target antigen elicits an immune response against Retrovirus. Retrovirus target antigens include those derived from an Oncovirus, a Lentivirus (e.g.HIV-I or HIV-2) or a Spumavirus. In some aspects, the target antigen elicits an immune response against Reovirus. Reovirus target antigens include, but are not limited to, those derived from an Orthoreovirus, a Rotavirus, an Orbivirus, or a Coltivirus. In some aspects, the target antigen elicits an immune response against Parvovirus, whose target antigens include those derived from Parvovirus B19.

In some aspects, the target antigen elicits an immune response against Herpesvirus, whose target antigens include those derived from a human herpesvirus, such as Herpes Simplex Viruses (HSV) (e.g.HSV types I and 2), Varicella-zoster virus (VZV), EpsteinBarr virus (EBV), Cytomegalovirus (CMV), Human Herpesvirus 6 (HHV6), Human Herpesvirus 7 (HHV7), and Human Herpesvirus 8 (HHV8).

In some aspects, the target antigen elicits an immune response against Papovaviruses, whose target antigens include those derived from Papillomaviruses and Adenovirus. In some aspects, the target antigen elicits an immune response to Chikungunya virus. I Some aspects, the target antigen elicits an immune response to Zika virus.

In some aspects, the target antigen elicits an immune response against a virus which infects fish.

Fungal target antigens may be derived from Dermatophytres and other opportunistic organisms.

In some aspects, the target antigen elicits an immune response against a parasite from the Plasmodium genus, such as P. falciparum, P. vivax, P. malariae or P. ovale. Thus the inventive compositions may be useful for immunizing against malaria. In some aspects, the target antigen elicits an immune response against a parasite from the Caligidae family, particularly those from the Lepeophtheirus and Caligus genera e.g., sea lice such as Lepeophtheirus salmonis or Caligus rogercresseyi.

In some aspects, the target antigen is a neoantigen specific to cancer cells or solid tumours. Peng, et al., Mol. Cancer, 18:128 (2019).

In some aspects the target antigen is a tumor antigen selected from: (a) cancer-testis antigens such as NY-ESO-I, SSX2, SCPI as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUMI (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLRFUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT I (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte antigens such as MART-1/Melan A, gp100, MCIR, melanocyte-stimulating hormone receptor, tyrosinase, tyrosinase related protein-I/TRPI and tyrosinase related protein-2/TRP2 (associated with, e.g., melanoma); (e) prostate associated antigens such as PAP, PSA, PSMA, PSH-PI, PSM-PI, PSM-P2, associated with e.g., prostate cancer; (f) immunoglobulin idiotypes (associated with myeloma and B cell lymphomas, for example). In certain aspects, tumor target antigens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23HI, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29&BCAA), CA 195, CA 242, CA-50, CAM43, CD68&KPI, CO-029, FGF-5, Ga733 (EpCAM), HTgp-175, M344, MA-50, MG7-Ag, MOV18, NB/70K, NY-CO-I, RCASI, SDCCAG16, TA-90 (Mac-2 binding protein/cyclophilin C-associated protein), TAAL6, TAG72, TLP, TPS, and the like.

In an aspect of the invention, the recombinant expression vector comprises, in order from 5′ to 3′, the following components: (a) the VEEV 5′ untranslated region (5′-UTR); (b) the nucleotide sequence encoding the VEEV non-structural proteins nsP1, nsP2, nsP3 and nsP4; (c) the VEEV 26S sub-genomic promoter; (d) the engineered MCS; (e) the VEEV 3′ untranslated region (3′-UTR); and (f) the nucleotide sequence encoding a VEEV poly A sequence. In an aspect of the invention, the MCS is positioned directly adjacent to the 5′ end or the 3′ end of the nucleotide sequence encoding the VEEV poly A sequence.

In an aspect of the invention, the recombinant expression vector comprises a vector backbone comprising one or more of ColE, an origin of replication (ori), a tet promoter, and one or more antibiotic resistance genes. In an aspect of the invention, the recombinant expression vector comprises a bacterial vector backbone or a modified bacterial vector backbone. In an aspect of the invention, the recombinant expression vector comprises a T7 promoter adjacent to the 5′ end of the 5′ UTR.

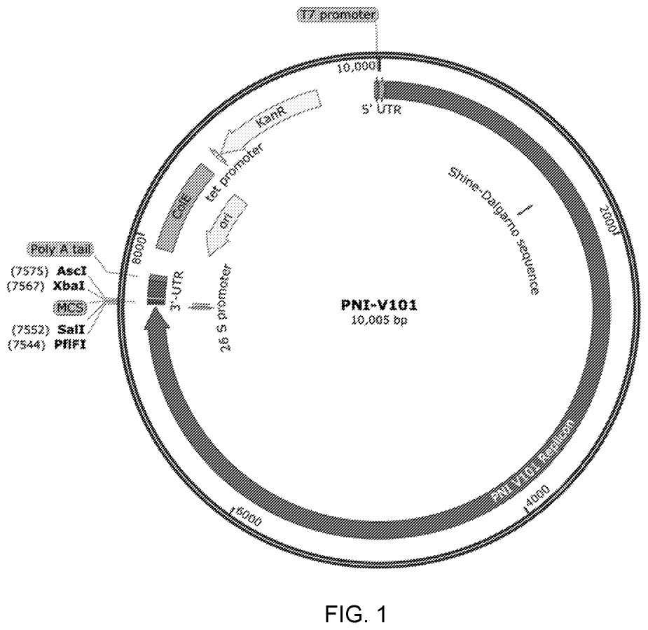

An example of a recombinant expression vector according to an aspect of the invention is shown in FIG. 1. FIG. 1 is a vector map of the vector named “PNI V101.” PNI V101 comprises a self-assembly replicon that may be used for any mRNA antigen. In a preferred aspect, the recombinant expression vector comprises a Venezuelan Equine Encephalitis Virus TC83 Replicon with a sub-genomic promoter containing a multiple cloning site to insert any GOI(s). The complete sequence of the PNI V101 cloning vector is set forth in SEQ ID NO: 1. In an aspect of the invention, the recombinant expression vector comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1.

In some aspects, the vector contains self-amplifying RNA (“saRNA”). In some aspects, nucleic acid refers to a vector including self-amplifying RNA. After in vivo administration of the recombinant expression vector, the delivered RNA is released and is translated inside a cell to provide the target antigen in situ. In certain aspects, the RNA is plus (“+”) stranded, so it can be translated by cells without needing any intervening replication steps such as reverse transcription. In certain aspects, the RNA is a self-replicating RNA. A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule is thus in certain aspects: a (+) strand molecule that can be directly translated after delivery to a cell, and this translation provides an RNA-dependent RNA polymerase which then produces both antisense and sense transcripts from the delivered RNA. Thus the delivered RNA leads to the production of multiple daughter RNAs. These daughter RNAs, as well as collinear sub-genomic transcripts, may be translated themselves to provide in situ expression of an encoded target antigen, or may be transcribed to provide further transcripts with the same sense as the delivered RNA which are translated to provide in situ expression of the target antigen. The overall result of this sequence of transcriptions is an amplification in the number of the introduced replicon RNAs, and so the encoded target antigen becomes a major polypeptide product of the host cells.

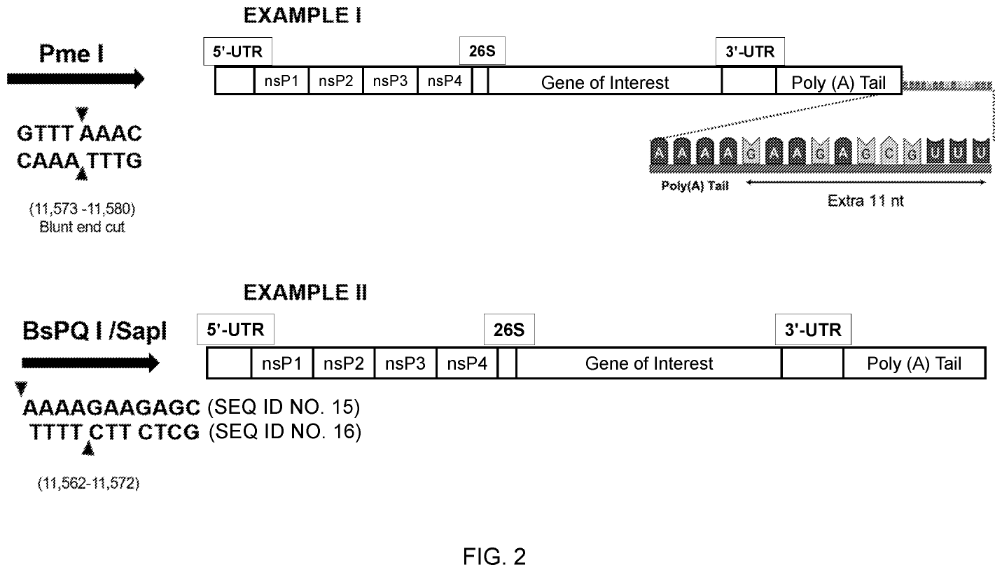

One suitable system for achieving self-replication is to use an alphavirus-based RNA replicon. These (+) stranded replicons are translated after delivery to a cell to yield a replicase (or replicase-transcriptase). The replicase is translated as a polyprotein which auto-cleaves to provide a replication complex which creates genomic (−) strand copies of the (+) strand delivered RNA. These (−) strand transcripts can themselves be transcribed to give further copies of the (+) stranded parent RNA, and also to give a sub-genomic transcript which encodes the target antigen. Translation of the sub-genomic transcript thus leads to in situ expression of the target antigen by the infected cell. Suitable alphavirus replicons can use a replicase from a Sindbis virus, a Semliki Forest virus, an eastern equine encephalitis virus, or more preferably, a Venezuelan equine encephalitis virus, etc. The system may be a hybrid or chimeric replicase in some aspects. FIG. 1 shows a replicon according to one aspect of the invention, showing a PNI V101 replicon capable of self-amplifying in mammalian cells and expressing, through mRNA assembled, immunogenic proteins such as SARS-COV-2 spike proteins. In FIG. 2, Pme I and BsPQI/Sap1 subtypes of the vaccine replicon are shown.

Mutant or wild-type virus sequences such as the attenuated TC83 mutant of VEEV can be used in replicons. A preferred self-replicating RNA molecule thus encodes (I) an RNA-dependent RNA polymerase which can transcribe RNA from the self-replicating RNA molecule and (ii) an target antigen. The polymerase can be an alphavirus replicase e.g. comprising one or more of alphavirus proteins nsPI, nsP2, nsP3 and nsP4. Whereas natural alphavirus genomes encode structural virion proteins in addition to the non-structural replicase polyprotein in particular aspects, a self-replicating RNA molecule of aspects of the invention does not encode alphavirus structural proteins. Thus a particular self-replicating RNA can lead to the production of genomic RNA copies of itself in a cell, but not to the production of RNA-containing virions. The alphavirus structural proteins which are necessary for perpetuation in wild-type viruses are absent from self-replicating RNAs of aspects of the invention and their place is taken by gene(s) encoding the target antigen of interest, such that the sub-genomic transcript encodes the target antigen rather than the structural alphavirus virion proteins. In later parts of the application, these two replicon types PME and BsPQI/Sap1 are noted by a “1^{st} Gen” or a “2^{nd} Gen” after the antigen.

Thus, a self-replicating RNA molecule useful with aspects of the invention may have two open reading frames: one encodes a replicase e.g., the first, (5′) open reading frame; the other open reading frame encodes an target antigen, e.g., the second, (3′) open reading frame. In some aspects, the RNA may have additional (e.g. downstream) open reading frames e.g. to encode further target antigens or to encode accessory polypeptides. A self-replicating RNA molecule can have a 5′ sequence which is compatible with the encoded replicase. Self-replicating RNA molecules can have various lengths, but they are typically about 5000-25000 nucleotides long e.g. 8000-15000 nucleotides, or 9000-12000 nucleotides. Thus, the RNA is longer than seen in conventional mRNA delivery. In some aspects, the self-replicating RNA is greater than about 2000 nucleotides, such as greater than about: 9,000, 12,000, 15,000, 18,000, 21,000, 24,000, or more nucleotides long.

Messenger RNA (mRNA) can be modified or unmodified, base modified, and may include different type of capping structures, such as Cap1. An RNA molecule may have a 5′ cap (e.g. a 7-methylguanosine). This cap can enhance in vivo translation of the RNA. The 5′ nucleotide of an RNA molecule useful with the invention may have a 5′ triphosphate group. In a capped RNA, this may be linked to a 7-methylguanosine via a 5′-to-5′ bridge. A 5′ triphosphate can enhance RIG-I binding and thus promote adjuvant effects. An RNA molecule may have a 3′ poly A tail. It may also include a poly A polymerase recognition sequence (e.g. AAUAAA) near its 3′ end. An RNA molecule useful with the invention for immunization purposes will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

RNA molecules can conveniently be prepared by in vitro transcription (IVT). IVT can use a (cDNA) template created and propagated in plasmid form in bacteria or created synthetically (for example by gene synthesis and/or polymerase chain-reaction (PCR) engineering methods). As discussed in WO2011/005799, the self-replicating RNA can include (in addition to any 5′ cap structure) one or more nucleotides having a modified nucleobase. For instance, a self-replicating RNA can include one or more modified pyrimidine nucleobases, such as pseudouridine and/or 5 methylcytosine residues. In some aspects, however, the RNA includes no modified nucleobases, and may include no modified nucleotides i.e., all of the nucleotides in the RNA are standard A, C, G and U ribonucleotides (except for any 5′ cap structure, which may include a 7′ methylguanosine). In other aspects, the RNA may include a 5′ cap comprising a 7′ methylguanosine, and the first 1, 2 or 3 5′ ribonucleotides may be methylated at the 2′ position of the ribose. An RNA may include only phosphodiester linkages between nucleosides, but in some aspects, it contains phosphoramidate, phosphorothioate, and/or methylphosphonate linkages. Multiple species of RNAs may be formulated within a single composition, such as two, three, four or more species of RNA, including different classes of RNA (such as mRNA, siRNA, self-replicating RNAs, and combinations thereof).

In accordance with one aspect of the invention, there is provided a vaccine including a replicon, a self-amplifying mRNA encoding a viral antigen and a synthetic lipid-based nanoparticle. In some aspects, the viral antigen incorporates a full-length codon optimized SARS-COV-2 spike protein sequence 3822 bp long targeting both original Wuhan strain of SARS-COV-2 virus as well as new variants of interests (VOIs) or variants of concern (VoCs) that arise from D614G dominant mutation of SARS-COV-2 virus. In some aspects, the viral antigen incorporates a full-length SARS-COV-2 spike protein sequence designs that are between 3813-4019 bp long targeting new variants of concern (VoCs) that arise from deletion and insertion mutations of SARS-COV-2 virus. In some aspects, the viral antigen incorporates full length SARS-COV-2 spike protein containing 63-71 bp leader sequences arising from N-terminal region of the mammalian tissue plasminogen activator (tPA), fibritin, fibronectin, or globulin proteins. In other aspects, the viral antigen incorporates a full-length codon optimized SARS-COV-2 spike protein sequence designs that are between 3813-4019 bp long targeting new variants of concern (VoCs) that arise from deletion and insertion mutations of SARS-COV-2 virus. In still other aspects, the viral antigen incorporates a full-length codon optimized SARS-COV-2 spike protein containing 63-71 bp leader sequences arising from N-terminal region of the mammalian tissue plasminogen activator (tPA), fibritin, fibronectin, or globulin proteins.

In aspects, the viral antigen incorporates full length codon optimized SARS-COV-2 spike protein containing 63-71 bp leader sequences arising from N-terminal region of the human and non-human primate tissue plasminogen activator (tPA), fibritin, fibronectin, or globulin proteins. In other aspects, the viral antigen incorporates codon-optimized truncated SARS-COV-2 spike protein sequence incorporating the N-terminal domain (NTD), Signal Sequence (SS), natural receptor binding domain (RBD), linker sequence, fibritin-foldon and transmembrane domain (TMD) to target SARS-COV-2 virus. In some aspects, the viral antigen incorporates codon-optimized truncated SARS-COV-2 spike protein sequence incorporating the N-terminal domain (NTD), Signal Sequence (SS), mutant receptor binding domain (mut-RBD), linker sequence, fibritin-foldon and transmembrane domain (TMD) to target new variants of interests (VoIs) and Variants of concerns (VoCs) that arise from deletion and/or insertion mutations of SARS-COV-2 viruses. In other aspects, the viral antigen incorporates codon-optimized truncated SARS-COV-2 spike protein sequence incorporating the N-terminal domain (NTD), Signal Sequence (SS), mutant receptor binding domain (mut-RBD), linker sequence, fibritin-foldon and transmembrane domain (TMD) to target new variants of interests (Vols) and Variants of concerns (VoCs) that arise from deletion and/or insertion mutations of SARS-COV-2 viruses.

In aspects of the invention, there is provided a vaccine wherein the viral antigen incorporates bicistronic gene cassette with truncated SARS-COV-2 spike protein sequence incorporating the N-terminal domain (NTD), Signal Sequence (SS), receptor binding domain (RBD), linker sequence, fibritin-foldon and transmembrane domain (TMD) with SAR-COV-2 Nucleoprotein sequence to target SARS-COV-2 virus

In aspects, the viral antigen incorporates bicistronic gene cassette with truncated SARS-COV-2 spike protein sequence incorporating the N-terminal domain (NTD), Signal Sequence (SS), mutant receptor binding domain (mut-RBD), linker sequence, fibritin-foldon and transmembrane domain (TMD) with SAR-COV-2 Nucleoprotein sequence to target new variants of interests (Vols) and Variants of concerns (VoCs) that arise from deletion and/or insertion mutations of SARS-COV-2 viruses. In aspects of the invention, the IVT transcribed self-amplifying mRNA based vaccine drug substance is purified using standard low shear rate tangential flow filtration or lithium chloride precipitation and resuspended in 0.5-2 mM Citrate buffer (pH 6.1-6.6). In still other aspects, the purified IVT transcribed self-amplifying mRNA based vaccine drug substance is resuspended in a cryoprotectant buffer composed of 0.5-2 mM Citrate buffer, 100 mM-500 mM Sucrose, 0.1-6% Mannitol (pH 6.1-6.6). In still other aspects, here is provided a vaccine wherein the purified IVT transcribed self-amplifying mRNA based vaccine drug substance is resuspended in a cryoprotectant buffer composed of 0.5-2 mM Citrate buffer, 100 mM-500 mM Sucrose, 0.1-6% Mannose (pH 6.1-6.6). In aspects, the purified IVT transcribed self-amplifying mRNA based vaccine drug substance resuspended in a cryoprotectant buffer composed of 0.5-2 mM Citrate buffer, 100 mM-500 mM Sucrose, 0.1-6% Mannitol (pH 6.1-6.6) freeze dried using primary drying temperature −45° C. to −60° C. and secondary drying temperature by ramping to 10° C.-25° C.

In aspects, the purified IVT transcribed self-amplifying mRNA based vaccine drug substance resuspended in a cryoprotectant buffer composed of 0.5-2 mM Citrate buffer, 100 mM-500 mM Sucrose, 0.1-6% Mannose (pH 6.1-6.6) freeze dried using primary drying temperature −45° C. to −60° C. and secondary drying temperature by ramping to 10° C.-25° C. In aspects of the invention, there is provided a synthetic lipid based nanoparticle comprises an ionizable lipid, a structural lipid, a sterol, and a stabilizer. In aspects, there is provided the described vaccine wherein a ratio of ionizable lipid to self-amplifying mRNA results in an N/P ratio of 4-9. Of 8. Of 6.

There is provided according to an aspect of the invention the vaccine described herein, wherein the vaccine is lyophilized.

The recombinant expression vectors of aspects of the invention may be provided with a pharmaceutically acceptable carrier. In an aspect of the invention, a pharmaceutical composition comprises the recombinant expression vector and a pharmaceutically acceptable carrier.

Pharmaceutically acceptable carriers for RNA vaccines may reduce or avoid degradation of the RNA vaccines by exonucleases and endonucleases in vivo. In an aspect of the invention, the pharmaceutically acceptable carrier is lipid nanoparticles (LNPs). LNPs may comprise a lipid or aqueous core surrounded by a lipid bilayer shell that is made of a combination of different lipids, each serving distinct functions. In an aspect of the invention, the lipid nanoparticle comprises: (a) an ionizable cationic lipid; (b) a structural lipid; (c) a stabilizer; and (d) a sterol. Ionizable cationic lipids spontaneously encapsulate negatively-charged mRNA VIA attractive electrostatic interactions with RNA and hydrophobic interactions. Structural lipids may reduce charge-related toxicity and maintain structure of the LNP. The sterol may stabilize the LNP and facilitate cell entry.

The properties of individual LNPs may be affected by how they are made. Diffusive or bulk mixing can lead to LNPs with variable compositions. Rapid mixing of the ethanol-lipid phase with mRNA in excess water may produce small, uniform LNPs. The Precision NanoSystems Inc. NANOASSEMBLR® line of mixers is recommended for producing LNPs.

The LNPs of aspects of the invention may be useful for the systemic or local delivery of recombinant expression vectors. The LNPs may have several advantages, including i) high biocompatibility and low toxicity in cell and tissue systems ii) relative ease of manufacture, and iii) an increased circulatory half-life in vivo due to their invisibility from the immune system.

Lipids are a structurally diverse group of organic compounds that are fatty acid derivatives or sterols. Lipids may include lipid like materials, such as lipidoids. Lipids are characterized by being insoluble in water but soluble in many organic solvents.

“Lipid mix compositions” refers to the types of components, ratios of components, and the ratio of the total components to the recombinant expression vector (nucleic acid payloads). For example, a lipid mix composition of 40 Mol % ionizable lipid, 20 Mol % structural lipid, 17 Mol % sterol, and 2.5 Mol % stabilizing agent would be a lipid mix composition. In preferred aspects, the lipid mix composition is 47.5 mol % IL/12.5 mol % DSPC/38.5 mol % Cholesterol/1.5 mol % PEG-DMG. In other preferred aspects, the 12.5 mol % DSPC is replaced with an equal amount of DOPE.

As used herein, “N/P” is the ratio of moles of the amine groups of ionizable lipids to those of the phosphate groups of nucleic acid. In aspects of the invention, N/P ratios are from 6 to 10, and most preferred ratios are from N/P 4-12. In one aspect the N/P ratio is 6, 8, or 10. The nucleic acid component is associated with this lipid mix composition to form a lipid nucleic acid particle, or LNP, in a premeditated ratio such as ionizable lipid amine (N) to nucleic acid phosphate ratio (P) of N/P 4, N/P 6, N/P 8, N/P 10, N/P 12 or another relevant particular N/P ratio. In preferred aspects, the N/P is 6.

Aspects of the invention provide LNPs (also referred to as “lipid particles,” “lipid nanoparticles,” or “lipid nucleic acid particles”) manufactured from the lipid mix compositions described herein. The LNP represents the physical organization of the lipid mix composition with the nucleic acid among the components. LNPs are generally spherical assemblies of lipids, nucleic acid, cholesterol, and stabilizing agents. Positive and negative charges, ratios, as well as hydrophilicity and hydrophobicity dictate the physical structure of the LNPs in terms of size and orientation of components. The structural organization of these lipid particles may lead to an aqueous interior with a minimum bilayer, as in liposomes, or it may have a solid interior, as in a solid nucleic acid lipid nanoparticle. There may be phospholipid monolayers or bilayers in single or multiple forms. Lipid particles may be from 1 to 1000 μm in size.

The compositions of the invention may comprise ionizable cationic lipids as a component. As used herein, the term “ionizable cationic lipid” refers to a lipid that is cationic or becomes ionizable (protonated) as the pH is lowered below the pKa of the ionizable group of the lipid but is more neutral at higher pH values. At pH values below the pKa, the lipid is then able to associate with negatively charged nucleic acids (e.g., oligonucleotides). As used herein, the term “ionizable cationic lipid” includes lipids that assume a positive charge on pH decrease from physiological pH, and any of a number of lipid species that carry a net positive charge at a selective pH, such as physiological pH. Examples of suitable ionizable cationic lipids are found in PCT Pub. Nos. WO20252589 and WO21000041. The ionizable cationic lipid may be present in LNPs in a ratio of 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, about 65 mol %, about 66 mol %, about 67 mol %, about 68 mol %, about 69 mol %, about 70 mol %, or a range defined by any two of the foregoing values (“Mol %” means the percentage of the total moles that is of a particular component), or a range defined by any two of the foregoing values. In an aspect of the invention, the lipid nanoparticle comprises from about 20 mol % to about 70 mol %, about 25 mol % to about 65 mol %, about 30 mol % to about 60 mol %, about 35 mol % to about 55 mol %, or about 40 mol % to about 50 mol % ionizable cationic lipid. DODMA, or 1,2-dioleyloxy-3-dimethylaminopropane, is an ionizable lipid, as is DLin-MC3-DMA or O-(Z,Z,Z,Z-heptatriaconta-6,9,26,29-tetraen-19-yl)-4-(N,N-dimethylamino) (“MC3”).

Structural lipids, also known as “helper lipids” or “neutral lipids” may be incorporated into LNPs of the invention in some aspects. The LNPs may include one or more structural lipids at about 5 mol % to about 45 mol %, about 10 mol % to about 40 mol %, about 15 mol % to about 35 mol %, about 20 mol % to about 30 mol %, about 10 to 40 Mol % of the LNP, or about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, or a range defined by any two of the foregoing values, of the LNP.

Suitable structural lipids may support the formation of LNPs during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides. Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE). In one preferred aspect, the structural lipid is distearoylphosphatidylcholine (DSPC). In preferred aspects, the structural lipid is DOPE. In preferred aspects, the structural lipid is DSPC.

In another aspect, the structural lipid is any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

“Stabilizer” or stabilizing agent is a term used to identify the agent that is added to the ionizable lipid, the structural lipid, and the sterol that form the lipid composition. Examples of non-ionic stabilizing agents include: Polysorbates (Tweens), Brij™ S20 (polyoxyethylene (20) stearyl ether), Brij™35 (Polyoxyethylene lauryl ether, Polyethyleneglycol lauryl ether), Brij™S10 (Polyethylene glycol octadecyl ether, Polyoxyethylene (10) stearyl ether), and Myrj™52 (polyoxyethylene (40) stearate).

In some aspects, the stabilizer is TPGS 1000 (D-α-Tocopherol polyethylene glycol 1000 succinate); or Tween 20/Polysorbate 80/Tridecyl-D-maltoside in equal ratios (called Lipid H in Table 15). In other preferred aspects, the stabilizing agent includes PEGylated lipids including PEG-DMG 2000 (“PEG-DMG”). Polyethylene glycol conjugated lipids may also be used. The stabilizing agent may be used alone or in combinations with each other.

In some aspects, the stabilizing agent includes about 0.1 to about 3 Mol % of the LNP. In some aspects, the stabilizing agent includes about 0.5 to about 2.5 Mol % of the LNP. In some aspects, the stabilizing agent is present at greater than about 2.5 Mol %. In some aspects the stabilizing agent is present at about 5 Mol %. In some aspects the stabilizing agent is present at about 10 Mol %. In some aspects, the stabilizing agent is present at a Mol % of about 0.2, about 0.3, about 0.4, about 0.5, about 0.6, about 0.7, about 0.8, about 0.9, about 1.0, about 1.1, about 1.2, about 1.3, about 1.4,1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.5, about 4.6, about 4.7, about 4.8, about 4.9, about 5.0 or a range defined by any two of the foregoing values, in the LNP. In other aspects, the stabilizing agent is about 2.6 to about 10 Mol % of the LNP. In other aspects, the stabilizing agent is present at greater than about 10 Mol % of the LNP. In an aspect, the lipid nanoparticle comprises from about 0.2 mol % to about 5 mol % stabilizer.

Sterols may be included in the LNP. Sterols include cholesterol, beta-sitosterol, and 20-alpha-hydroxysterol, and phytosterol. In some aspects, sterol is present at about 30 to about 50 Mol % of the final lipid mix in some aspects. Alternately cholesterol is present at about 35 to about 41 Mol % of the final lipid mix. In some aspects sterol is present at about 17 mol % to about 38.5 mol %. In other aspects, sterol is absent. In some aspects a modified sterol or synthetically derived sterol is present. In an aspect, the lipid nanoparticle comprises from about 15 mol % to about 45 mol %, about 20 mol % to about 40 mol %, about 25 mol % to about 35 mol % sterol. In an aspect, the sterol is present at 15 mol %, about 16 mol %, about 17 mol %, about 18 mol %, about 19 mol %, about 20 mol %, about 21 mol %, about 22 mol %, about 23 mol %, about 24 mol %, about 25 mol %, 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, or a range defined by any two of the foregoing values, in the LNP.

The LNPs of aspects of the invention can be assessed for size using devices that size particles in solution, such as the Malvern™ Zetasizer™. In an aspect of the invention, the LNP have a size from about 50 nm to about 130 nm, about 55 nm to about 125 nm, about 60 nm to about 120 nm, about 65 nm to about 115 nm, about 70 nm to about 110 nm, about 75 nm to about 100 nm, or about 80 nm to about 100 nm. In some aspects, the LNP have a size of about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, or a range defined by any two of the foregoing values. Smaller particles generally exhibit increased circulatory lifetime in vivo compared to larger particles. Smaller particles have an increased ability to reach tumor sites than larger nanoparticles.

The lipid particles according to aspects of the invention can be prepared by standard T-tube mixing techniques, turbulent mixing, trituration mixing, agitation promoting orders self-assembly, or passive mixing of all the elements with self-assembly of elements into nanoparticles. A variety of methods have been developed to formulate lipid nanoparticles (LNP) containing genetic drugs. Suitable methods are disclosed in U.S. Pat. Nos. 5,753,613 and 6,734,171, by way of example. These methods include mixing preformed lipid particles with nucleic acid in the presence of ethanol or mixing lipid dissolved in ethanol with an aqueous media containing nucleic acid and result in lipid particles with nucleic acid encapsulation efficiencies of 65-99%. Both of these methods rely on the presence of ionizable lipid to achieve encapsulation of nucleic acid and a stabilizing agent to inhibit aggregation and the formation of large structures. The properties of the lipid particle systems produced, including size and nucleic acid encapsulation efficiency, are sensitive to a variety of lipid mix composition parameters such as ionic strength, lipid and ethanol concentration, pH, nucleic acid concentration and mixing rates.

Microfluidic two-phase droplet techniques have been applied to produce monodisperse polymeric microparticles for drug delivery or to produce large vesicles for the encapsulation of cells, proteins, or other biomolecules. The use of hydrodynamic flow focusing, a common microfluidic technique to provide rapid mixing of reagents, to create monodisperse liposomes of controlled size has been demonstrated.

In general, parameters such as the relative lipid and nucleic acid concentrations at the time of mixing, as well as the mixing rates may be difficult to control using current formulation procedures, resulting in variability in the characteristics of nucleic acid produced, both within and between preparations. Automatic micro-mixing instruments such as the NanoAssemblr® instruments (Precision NanoSystems Inc, Vancouver, Canada) provide the rapid and controlled manufacture of nanomedicines (liposomes, lipid nanoparticles, and polymeric nanoparticles). NanoAssemblr® instruments may accomplish controlled molecular self-assembly of nanoparticles via microfluidic mixing cartridges that allow millisecond mixing of nanoparticle components at the nanoliter, microlitre, or larger scale with customization or parallelization. Rapid mixing on a small scale allows reproducible control over particle synthesis and quality that is not possible in larger instruments.

Preferred methods incorporate microfluidic mixing devices like the NanoAssemblr® Spark™, Ignite™, Benchtop™ and NanoAssemblr® Blaze™ instruments in order to encapsulate nearly 100% of the nucleic acid in one step. In one aspect, the lipid particles are prepared by a process by which from about 90 to about 100% of the nucleic acid used in the formation process is encapsulated in the particles.

U.S. Pat. Nos. 9,758,795 and 9,943,846, describe methods of using small volume mixing technology and novel formulations derived thereby. U.S. Pat. No. U.S. Pat. No. 10,159,652 describes more advanced methods of using small volume mixing technology and products to formulate different materials. U.S. Pat. No. 9,943,846 by Walsh, et al. discloses microfluidic mixers with different paths and wells to elements to be mixed. PCT Pub. No. WO 2017117647 by Wild, Leaver and Taylor discloses microfluidic mixers with disposable sterile paths. U.S. Pat. No. 10,076,730 by Wild, Leaver and Taylor discloses bifurcating toroidal micromixing geometries and their application to microfluidic mixing. PCT Pub. No. WO2018006166 by Chang, Klaassen, Leaver et al. discloses a programmable automated micromixer and mixing chips therefore. U.S. Design Nos. D771834, D771833, D772427, and D803416 by Wild and Leaver, and U.S. Design Nos. D800335, D800336 and D812242 by Chang et al., disclose mixing cartridges having microchannels and mixing geometries for mixer instruments sold by Precision NanoSystems Inc.

In aspects of the invention, devices for biological microfluidic mixing are used to prepare the lipid particles according to aspects of the invention. The devices include a first and second stream of reagents, which feed into the microfluidic mixer, and lipid particles are collected from the outlet, or emerge into a sterile environment.

The first stream includes a nucleic acid in a first solvent. Suitable first solvents include solvents in which the nucleic acids are soluble and that are miscible with the second solvent. Suitable first solvents include aqueous buffers. Representative first solvents include citrate and acetate buffers or other low pH buffers.

The second stream includes lipid mix materials in a second solvent. Suitable second solvents include solvents in which the ionizable lipids according to aspects of the invention are soluble, and that are miscible with the first solvent. Suitable second solvents include 1,4-dioxane, tetrahydrofuran, acetone, acetonitrile, dimethyl sulfoxide, dimethylformamide, acids, and alcohols. Representative second solvents include aqueous ethanol 90%, or anhydrous ethanol.

In one aspect of the invention, a suitable device includes one or more microchannels (i.e., a channel having its greatest dimension less than 1 millimeter). In one example, the microchannel has a diameter from about 20 to about 300 μm. In examples, at least one region of the microchannel has a principal flow direction and one or more surfaces having at least one groove or protrusion defined therein, the groove or protrusion having an orientation that forms an angle with the principal direction (e.g., a staggered herringbone mixer), as described in U.S. Pat. No. 9,943,846, or a bifurcating toroidal flow as described in U.S. Pat. No. 10,076,730. To achieve maximal mixing rates, it may be advantageous to avoid undue fluidic resistance prior to the mixing region. Thus, one example of a device has non-microfluidic channels having dimensions greater than 1000 μm, to deliver the fluids to a single mixing channel.

Particle sizes and “polydispersity index” (PDI) of the lipid particle can be measured by dynamic light scattering (DLS). PDI indicates the width of the particle distribution. This is a parameter calculated from a cumulative analysis of the (DLS)-measured intensity autocorrelation function assuming a single particle size mode and a single exponential fit to the autocorrelation function. From a biophysical point of view, a PDI below 0.1 indicates that the sample is monodisperse. The particles produced by mechanical micromixers such as the NanoAssemblr® Spark™ and NanoAssemblr® Ignite™ (Precision NanoSystems Inc.) are substantially homogeneous in size assuming all other variables are neutral. A lower PDI indicates a more homogenous population of lipid particles. The Spark™ instrument is used in a screening setting to identify the lead compositions. Once the composition is selected, the lipid particle can be fine-tuned using the NanoAssemblr® Ignite™ instrument. Once the process parameters Flow Rate Ratio and Total Flow Rate are identified for a specific nanoparticle composition, the nanoparticle technology can be scaled up using the same process parameter values.

Less complex mixing methods and instruments such as those disclosed in U.S. Published Patent Application US20040262223 are also useful in creating lipid particle compositions of the invention.

In certain aspects, the present invention provides methods for introducing a nucleic acid into a cell (i.e. transfection). In this disclosure, “transfection” means the transfer of nucleic acid into cells for the purpose of inducing the expression of a specific gene(s) of interest in both laboratory and clinical settings. It typically includes an ionizable lipid to associate with nucleic acid, and structural lipids. LIPOFECTIN™ and LIPOFECTAMINE™ are established commercial transfecting reagents sold by ThermoFisher Scientific. These research reagents contain permanently cationic lipid/s and are not suitable for use in or ex vivo.

Transfection efficiency is commonly defined as either the i) percentage of cells in the total treated population showing positive expression of the delivered gene, as measured by live or fixed cell imaging (for detection of fluorescent protein), and flow cytometry or ii) the intensity or amount of protein expressed by treated cell(s) as analyzed by live or fixed cell imaging or flow cytometry or iii) using protein quantification techniques such as ELISA, or western blot. These methods may be carried out by contacting the particles or lipid mix compositions of the present invention with the cells for a period of time sufficient for intracellular delivery to occur.

For in vivo administration, the pharmaceutical compositions are preferably administered parenterally (e.g., intraarticularly, intravenously, intraperitoneally, subcutaneously, intrathecally, intradermally, intratracheally, intraosseous, intramuscularly, intratumorally, or to the interstitial space of a tissue). In particular aspects, the pharmaceutical compositions are administered intravenously, intrathecally, or intraperitoneally by a bolus injection. Alternative delivery routes include rectal, oral (e.g. tablet, drops, spray), buccal, sublingual, vaginal, topical skin, eyes, mucus membranes), transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. A typical intramuscular dose is 0.5 ml.

In another example, the lipid mix compositions of the invention can be used for delivery of nucleic acids to a sample of patient cells that are ex vivo, then are returned to the patient.

The compositions of the invention may be useful for immunization. For immunization purposes, a composition of the invention will generally be prepared as an injectable, a pulmonary or nasal aerosol, or in a delivery device (e.g. syringe, nebulizer, sprayer, inhaler, dermal patch, etc.). This delivery device can be used to administer a pharmaceutical composition to a subject, e.g. to a human, for immunization.

The RNA may be delivered with a lipid composition of the invention (e.g. formulated as a liposome or LNP). In some aspects, the invention utilizes LNPs within which target antigen-encoding RNA is encapsulated. Encapsulation within LNPs can protect RNA from RNase digestion. The encapsulation efficiency does not have to be 100%. Presence of external RNA molecules (e.g. on the exterior surface of a liposome or LNP) or “naked” RNA molecules (RNA molecules not associated with a liposome or LNP) is acceptable. Preferably, for a composition comprising lipids and RNA molecules, at least half of the RNA molecules (e.g., at least 50%, at least 60%, at least 65%, at least 70%, at least 75%, at least 80%, at least 85%, at least 90%, at least 95%, at least about 96%, at least about 97%, at least about 98%, or at least 99% of the RNA molecules) are encapsulated in LNPs or complexed LNPs.

For delivery of target antigen-coding RNA, the preferred range of LNP diameters is in the range of 60-180 nm, and in more particular aspects, in the range of 80-160 nm. An LNP can be part of a composition comprising a population of LNPs, and the LNPs within the population can have a range of diameters. For a composition comprising a population of LNPs with different diameters, it is preferred that (I) at least 80% by number of the LNPS have diameters in the range of 60-180 nm, e.g., in the range of 80-160 nm, (ii) the average diameter (by intensity, e.g. Z-average) of the population is ideally in the range of 60-180 nm, e.g., in the range of 80-160 nm; and/or the diameters within the plurality have a polydispersity index <0.2. To obtain LNPs with the desired diameter(s), mixing can be performed using a process in which two feed streams of aqueous RNA solution are combined in a single mixing zone with one stream of an ethanolic lipid solution, all at the same flow rate e.g. in a microfluidic channel. See other description relating to NanoAssemblr® microfluidic mixers sold by Precision NanoSystems Inc., Vancouver, Canada.

In certain aspects, the lipid compositions provided by the invention (such as LNPS) have adjuvant activity, i.e., in the absence of an target antigen, such as protein antigen or a nucleic acid (DNA or RNA), such as a nucleic acid encoding such an antigen.

A pharmaceutical composition of the invention, particularly one useful for immunization, may include one or more small molecule immunopotentiators. Pharmaceutical compositions of the invention may include one or more preservatives, such as thiomersal or 2 phenoxyethanol. Mercury-free and preservative-free vaccines can be prepared.

Compositions comprise an effective amount of the lipid compositions described herein (e.g., LNP), as well as any other components, as needed. Immunologically effective amount refers to the amount administered to an individual, either in a single dose or as part of a series, that is effective for treatment (e.g., prophylactic immune response against a pathogen). This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The compositions of the invention will generally be expressed in terms of the amount of RNA per dose. A preferred dose has ˜1000 pg RNA (e.g. from 10-1000 pg, such as about 10 μg, 25 pg, 50 μg, 75 μg or 100 pg, etc.), but expression can be seen at much lower levels e.g.˜1 μg/dose, ˜100 ng/dose, ˜10 ng/dose, ˜1 ng/dose, etc. In some aspects, the preferred dose is 100 μg. In other aspects, the preferred dose is up to 250 μg. The invention also provides a delivery device (e.g. syringe, nebulizer, sprayer, inhaler, dermal patch, etc.) containing a pharmaceutical composition of the invention. This device can be used to administer the composition to a vertebrate subject.

The LNP-formulated RNA and pharmaceutical compositions described herein are for in vivo use for inducing an immune response against an target antigen of interest. The invention provides a method for inducing an immune response in a vertebrate comprising administering an effective amount of the LNP formulated RNA, or pharmaceutical composition, as described herein. The immune response is preferably protective and preferably involves antibodies and/or cell-mediated immunity. The compositions may be used for both priming and boosting purposes. Alternatively, a prime-boost immunization schedule can be a mix of RNA and the corresponding polypeptide target antigen (e.g., RNA prime, protein boost).

An aspect of the invention also provides an LNP or pharmaceutical composition for use in inducing an immune response in a vertebrate. The invention also provides the use of a LNP or pharmaceutical composition in the manufacture of a medicament for inducing an immune response in a vertebrate. By inducing an immune response in the vertebrate by these uses and methods, the vertebrate can be protected against various diseases and/or infections e.g. against bacterial and/or viral diseases as discussed above. Vaccines according to the invention may either be prophylactic (i.e. to prevent infection) or therapeutic (i.e. to treat infection), but will typically be prophylactic. The vertebrate is preferably a mammal, such as a human or a large veterinary mammal (e.g. horses, cattle, deer, sheep, llamas, goats, pigs).

The invention may be used to induce systemic and/or mucosal immunity, preferably to elicit an enhanced systemic and/or mucosal immunity. Dosage can be by a single dose schedule or a multiple dose schedule. Multiple doses may be used in a primary immunization schedule and/or in a booster immunization schedule.

In a multiple dose schedule the various doses may be given by the same or different routes, for example, a parenteral prime and mucosal boost, a mucosal prime and parenteral boost, etc. Multiple doses will typically be administered at least one week apart (e.g. about two weeks, about three weeks, about four weeks, about six weeks, about eight weeks, about ten weeks, about 12 weeks, about 16 weeks, etc.). In one aspect, multiple doses may be administered approximately six weeks, ten weeks and 14 weeks after birth, e.g. at an age of six weeks, ten weeks and 14 weeks, as often used in the World Health Organization's Expanded Program on Immunization (“EPI”). In an alternative aspect, two primary doses are administered about two months apart, e.g. about seven, eight or nine weeks apart, followed by one or more booster doses about six months to one year after the second primary dose, e.g. about six, eight, ten or 12 months after the second primary dose. In a further aspect, three primary doses are administered about two months apart, e.g. about seven, eight or nine weeks apart, followed by one or more booster doses about six months to one year after the third primary dose.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the nucleic acid, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams and Wilkins, Baltimore, MD, 2006). The use of a conventional excipient medium is contemplated herein, except insofar as any conventional excipient medium may be incompatible with a substance or its derivatives, such as by producing any undesirable biological effect or otherwise interacting in a deleterious manner with any other component(s) of the pharmaceutical composition.

In some aspects, the particle size of the lipid particles may be increased and/or decreased. The change in particle size may be able to help counter biological reaction such as, but not limited to, inflammation or may increase the biological effect of the NAT delivered to mammals by changing biodistribution. Size may also be used to determine target tissue, with larger particles being cleared quickly and smaller one reaching different organ systems.

The following examples further illustrate the invention but, of course, should not be construed as in any way limiting its scope.

### EXAMPLES

All solvents and reagents described in the Examples were commercial products and used as such unless noted otherwise. Temperatures are given in degrees Celsius.

### Abbreviations and Terms

Deg. C=Degree Celsius
EPO=erythropoietin
g=gram
GOI= “Gene of interest”
h=hour(s)
HPLC=High performance liquid chromatography
IL=ionizable lipid
MFI=Median Fluorescence Intensity
min=minute(s)
mL=milliliter(s)
mmol=millimole(s)
PBS=phosphate buffered solution
Stabilizing Agent=any stabilizing agent including PEG-DMG 2000
Tween20=polysorbate 20
Tween80=Polysorbate 80
ug=μg
wt=weight

Components of the Lipid Mixes include the ionizable lipid, structural lipid, cholesterol, and stabilizing agent. Low pH buffers (3-6) may be used. For ionizable aminolipids, the pH of the buffer is typically below the pKa of the lipid.

GOI signifies a genetic element or elements intended for expression to achieve a therapeutic goal, including immunization. A1-A19 SARS Cov-2 antigenic coding elements are GOI in aspects of the present invention.

IL is a lipid that is cationic at higher pH and converts to uncharged at lower pH.

PNI V101 nCOV is a replicon with SARS nCOV-2 antigenicity built in.

PNI V101 nCOV PNI A5 saRNA is a particular A5 antigenic type of the SARS nCOV-2 vaccine.

PNI V101 nCOV PNI A5 saRNA PNI 516 LNP is the A5 antigen type in a PNI 516 ionizable lipid nanoparticle.

PNI 516-VACCMIX-A-LM is a particular lipid mix comprising (Z)-3-(2-((1,17-bis (2-octylcyclopropyl) heptadecan-9-yl)oxy)-2-oxoethyl)-2-(pent-2-en-1-yl)cyclopentyl 4-(dimethylamino) butanoate at 47.5 mol % with 12.5 mol % DOPE, 38.5 mol % cholesterol, and 1.5 mol % PEG-DMG 2000.

VACCMIX-A describes any formulation with 47.5 mol % ionizable lipid with 12.5 mol % structural lipid, 38.5 mol % cholesterol, and 1.5 mol % PEG-DMG 2000. The ionizable lipid and structural lipid are specified.

### Example 1

This example demonstrates a method of preparing a recombinant expression vector according to an aspect of the invention.

Method for Self-Amplifying mRNA Synthesis.

The restriction digestion of the circular plasmid encoding SARS COV 2 spike protein was carried out according to manufacturer's instructions for BspQI (New England BioLabs Inc., Catalog number R0712S) or Pmel (New England BioLabs Inc., Catalog number R0560S), in prescribed buffers.

The linearized vector was purified using Phenol/Chloroform/Isoamyl alcohol (25:24:1) and sodium acetate precipitation. Briefly, equal volumes of Phenol/Chloroform/Isoamyl alcohol solution were added to the linearized vector, vortexed for 20 seconds and incubated at room temperature for 2 minutes. The mixture was spun down at 16,000g for 5 minutes at room temperature, after which the top aqueous phase containing the linearized vector was carefully pipetted into a clean RNase/DNase free tube and precipitated using 0.3M sodium acetate and glycogen. Three (3) volumes of 100% ethanol were added, mixed well and incubated in −20 deg C. freezer overnight. The next day, the mixture was spun down at maximum speed at 4 deg C, and the pellet washed twice with ice cold 70% ethanol. The ethanol was removed carefully, and the DNA pellet was air-dried and resuspended in nuclease free water. The concentration and purity of the linearized vector was checked using NANODROP spectrophotometer (Thermo Fisher Scientific, Waltham, MA).

In vitro transcription was carried out using HiScribe™ T7 High Yield RNA Synthesis Kit (New England BioLabs, Inc., Catalog number E2040S). This was followed by linear DNA template digestion performed using TURBO DNase (Thermofisher Scientific, Catalog number AM2238). The final, in vitro-transcribed, self-amplifying RNA (saRNA) was capped using Vaccina Capping System (New England Biolabs Inc, Catalog number M2080S). All of these processes were performed according to the manufacturer's protocol to generate the self-amplifying RNA utilizing the DNA templates obtained from the vector linearization strategy. The purification of the capped saRNA was performed using standard LiCl precipitation, followed by 70% ethanol wash and resuspension of the RNA pellet in the RNA storage solution (Thermofisher).

### Cloning Vector Design and Linearization Strategy for PNI V101 Vector

The PNI V101 cloning vector is a 10,005 base pair (bp) synthetic plasmid DNA that incorporates genes encoding a single polyprotein viral RNA replication machinery of alphavirus subfamily: Venezuelan Equine Encephalitis Virus (VEEV). The viral RNA replication machinery includes the VEEV 5′ untranslated region (5′-UTR), and non-structural proteins (nsP1, nsP2, nsP3 and nsP4), together with a 26S sub-genomic promoter from TC83 strain of VEEV with an engineered multiple cloning site (MCS) located at 7541-7593 bp which allow for seamless insertion of the gene of interest followed by 3′ untranslated region (3′-UTR) and 38-40 bp poly A sequences encoded into a modified bacterial pUC57 vector encoding ColE, origin of replication (ori), tet promoter, synthetic AmpR and KanR genes. The T7 promoter is encoded at the 5′ region of the 5′-UTR sequences. The complete sequence of the PNI V101 cloning vector is set forth in SEQ ID NO: 1 in Table 1. The underlined region of the sequence represents the location of the engineered multiple cloning site (MCS).

TABLE 1
SEQ ID NO: 1
Complete Sequence and Map of PNI V101 Cloning Vector

ATAGGCGGCGCATGAGAGAAGCCCAGACCAATTACCTACCCAAAATGGAGAAAGTT
CACGTTGACATCGAGGAAGACAGCCCATTCCTCAGAGCTTTGCAGCGGAGCTTCCC
GCAGTTTGAGGTAGAAGCCAAGCAGGTCACTGATAATGACCATGCTAATGCCAGAG
CGTTTTCGCATCTGGCTTCAAAACTGATCGAAACGGAGGTGGACCCATCCGACACG
ATCCTTGACATTGGAAGTGCGCCCGCCCGCAGAATGTATTCTAAGCACAAGTATCAT
TGTATCTGTCCGATGAGATGTGCGGAAGATCCGGACAGATTGTATAAGTATGCAACT
AAGCTGAAGAAAAACTGTAAGGAAATAACTGATAAGGAATTGGACAAGAAAATGAAG
GAGCTCGCCGCCGTCATGAGCGACCCTGACCTGGAAACTGAGACTATGTGCCTCCA
CGACGACGAGTCGTGTCGCTACGAAGGGCAAGTCGCTGTTTACCAGGATGTATACG
CGGTTGACGGACCGACAAGTCTCTATCACCAAGCCAATAAGGGAGTTAGAGTCGCC
TACTGGATAGGCTTTGACACCACCCCTTTTATGTTTAAGAACTTGGCTGGAGCATATC
CATCATACTCTACCAACTGGGCCGACGAAACCGTGTTAACGGCTCGTAACATAGGCC
TATGCAGCTCTGACGTTATGGAGCGGTCACGTAGAGGGATGTCCATTCTTAGAAAGA
AGTATTTGAAACCATCCAACAATGTTCTATTCTCTGTTGGCTCGACCATCTACCACGA
GAAGAGGGACTTACTGAGGAGCTGGCACCTGCCGTCTGTATTTCACTTACGTGGCA
AGCAAAATTACACATGTCGGTGTGAGACTATAGTTAGTTGCGACGGGTACGTCGTTA
AAAGAATAGCTATCAGTCCAGGCCTGTATGGGAAGCCTTCAGGCTATGCTGCTACGA
TGCACCGCGAGGGATTCTTGTGCTGCAAAGTGACAGACACATTGAACGGGGAGAGG
GTCTCTTTTCCCGTGTGCACGTATGTGCCAGCTACATTGTGTGACCAAATGACTGGC
ATACTGGCAACAGATGTCAGTGCGGACGACGCGCAAAAACTGCTGGTTGGGCTCAA
CCAGCGTATAGTCGTCAACGGTCGCACCCAGAGAAACACCAATACCATGAAAAATTA
CCTTTTGCCCGTAGTGGCCCAGGCATTTGCTAGGTGGGCAAAGGAATATAAGGAAG
ATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAA
CCATCATCAAAGTGAACAGCGATTTCCACTCATTCGTGCTGCCCAGGATAGGCAGTA
ACACATTGGAGATCGGGCTGAGAACAAGAATCAGGAAAATGTTAGAGGAGCACAAG
GAGCCGTCACCTCTCATTACCGCCGAGGACGTACAAGAAGCTAAGTGCGCAGCCGA
TGAGGCTAAGGAGGTGCGTGAAGCCGAGGAGTTGCGCGCAGCTCTACCACCTTTG
GCAGCTGATGTTGAGGAGCCCACTCTGGAAGCCGATGTAGACTTGATGTTACAAGA
GGCTGGGGCCGGCTCAGTGGAGACACCTCGTGGCTTGATAAAGGTTACCAGCTACG
ATGGCGAGGACAAGATCGGCTCTTACGCTGTGCTTTCTCCGCAGGCTGTACTCAAG
AGTGAAAAATTATCTTGCATCCACCCTCTCGCTGAACAAGTCATAGTGATAACACACT
CTGGCCGAAAAGGGCGTTATGCCGTGGAACCATACCATGGTAAAGTAGTGGTGCCA
GAGGGACATGCAATACCCGTCCAGGACTTTCAAGCTCTGAGTGAAAGTGCCACCATT
GTGTACAACGAACGTGAGTTCGTAAACAGGTACCTGCACCATATTGCCACACATGGA
GGAGCGCTGAACACTGATGAAGAATATTACAAAACTGTCAAGCCCAGCGAGCACGA
CGGCGAATACCTGTACGACATCGACAGGAAACAGTGCGTCAAGAAAGAACTAGTCA
CTGGGCTAGGGCTCACAGGCGAGCTGGTGGATCCTCCCTTCCATGAATTCGCCTAC
GAGAGTCTGAGAACACGACCAGCCGCTCCTTACCAAGTACCAACCATAGGGGTGTA
TGGCGTGCCAGGATCAGGCAAGTCTGGCATCATTAAAAGCGCAGTCACCAAAAAAG
ATCTAGTGGTGAGCGCCAAGAAAGAAAACTGTGCAGAAATTATAAGGGACGTCAAGA
AAATGAAAGGGCTGGACGTCAATGCCAGAACTGTGGACTCAGTGCTCTTGAATGGAT
GCAAACACCCCGTAGAGACCCTGTATATTGACGAAGCTTTTGCTTGTCATGCAGGTA
CTCTCAGAGCGCTCATAGCCATTATAAGACCTAAAAAGGCAGTGCTCTGCGGGGATC
CCAAACAGTGCGGTTTTTTTAACATGATGTGCCTGAAAGTGCATTTTAACCACGAGAT
TTGCACACAAGTCTTCCACAAAAGCATCTCTCGCCGTTGCACTAAATCTGTGACTTC
GGTCGTCTCAACCTTGTTTTACGACAAAAAAATGAGAACGACGAATCCGAAAGAGAC
TAAGATTGTGATTGACACTACCGGCAGTACCAAACCTAAGCAGGACGATCTCATTCT
CACTTGTTTCAGAGGGTGGGTGAAGCAGTTGCAAATAGATTACAAAGGCAACGAAAT
AATGACGGCAGCTGCCTCTCAAGGGCTGACCCGTAAAGGTGTGTATGCCGTTCGGT
ACAAGGTGAATGAAAATCCTCTGTACGCACCCACCTCAGAACATGTGAACGTCCTAC
TGACCCGCACGGAGGACCGCATCGTGTGGAAAACACTAGCCGGCGACCCATGGAT
AAAAACACTGACTGCCAAGTACCCTGGGAATTTCACTGCCACGATAGAGGAGTGGC
AAGCAGAGCATGATGCCATCATGAGGCACATCTTGGAGAGACCGGACCCTACCGAC
GTCTTCCAGAATAAGGCAAACGTGTGTTGGGCCAAGGCTTTAGTGCCGGTGCTGAA
GACCGCTGGCATAGACATGACCACTGAACAATGGAACACTGTGGATTATTTTGAAAC
GGACAAAGCTCACTCAGCAGAGATAGTATTGAACCAACTATGCGTGAGGTTCTTTGG
ACTCGATCTGGACTCCGGTCTATTTTCTGCACCCACTGTTCCGTTATCCATTAGGAAT
AATCACTGGGATAACTCCCCGTCGCCTAACATGTACGGGCTGAATAAAGAAGTGGTC
CGTCAGCTCTCTCGCAGGTACCCACAACTGCCTCGGGCAGTTGCCACTGGAAGAGT
CTATGACATGAACACTGGTACACTGCGCAATTATGATCCGCGCATAAACCTAGTACC
TGTAAACAGAAGACTGCCTCATGCTTTAGTCCTCCACCATAATGAACACCCACAGAG
TGACTTTTCTTCATTCGTCAGCAAATTGAAGGGCAGAACTGTCCTGGTGGTCGGGGA
AAAGTTGTCCGTCCCAGGCAAAATGGTTGACTGGTTGTCAGACCGGCCTGAGGCTA
CCTTCAGAGCTCGGCTGGATTTAGGCATCCCAGGTGATGTGCCCAAATATGACATAA
TATTTGTTAATGTGAGGACCCCATATAAATACCATCACTATCAGCAGTGTGAAGACCA
TGCCATTAAGCTTAGCATGTTGACCAAGAAAGCTTGTCTGCATCTGAATCCCGGCGG
AACCTGTGTCAGCATAGGTTATGGTTACGCTGACAGGGCCAGCGAAAGCATCATTG
GTGCTATAGCGCGGCAGTTCAAGTTTTCCCGGGTATGCAAACCGAAATCCTCACTTG
AAGAGACGGAAGTTCTGTTTGTATTCATTGGGTACGATCGCAAGGCCCGTACGCACA
ATCCTTACAAGCTTTCATCAACCTTGACCAACATTTATACAGGTTCCAGACTCCACGA
AGCCGGATGTGCACCCTCATATCATGTGGTGCGAGGGGATATTGCCACGGCCACCG
AAGGAGTGATTATAAATGCTGCTAACAGCAAAGGACAACCTGGCGGAGGGGTGTGC
GGAGCGCTGTATAAGAAATTCCCGGAAAGCTTCGATTTACAGCCGATCGAAGTAGGA
AAAGCGCGACTGGTCAAAGGTGCAGCTAAACATATCATTCATGCCGTAGGACCAAAC
TTCAACAAAGTTTCGGAGGTTGAAGGTGACAAACAGTTGGCAGAGGCTTATGAGTCC
ATCGCTAAGATTGTCAACGATAACAATTACAAGTCAGTAGCGATTCCACTGTTGTCCA
CCGGCATCTTTTCCGGGAACAAAGATCGACTAACCCAATCATTGAACCATTTGCTGA
CAGCTTTAGACACCACTGATGCAGATGTAGCCATATACTGCAGGGACAAGAAATGGG
AAATGACTCTCAAGGAAGCAGTGGCTAGGAGAGAAGCAGTGGAGGAGATATGCATA
TCCGACGACTCTTCAGTGACAGAACCTGATGCAGAGCTGGTGAGGGTGCATCCGAA
GAGTTCTTTGGCTGGAAGGAAGGGCTACAGCACAAGCGATGGCAAAACTTTCTCATA
TTTGGAAGGGACCAAGTTTCACCAGGCGGCCAAGGATATAGCAGAAATTAATGCCAT
GTGGCCCGTTGCAACGGAGGCCAATGAGCAGGTATGCATGTATATCCTCGGAGAAA
GCATGAGCAGTATTAGGTCGAAATGCCCCGTCGAAGAGTCGGAAGCCTCCACACCA
CCTAGCACGCTGCCTTGCTTGTGCATCCATGCCATGACTCCAGAAAGAGTACAGCG
CCTAAAAGCCTCACGTCCAGAACAAATTACTGTGTGCTCATCCTTTCCATTGCCGAA
GTATAGAATCACTGGTGTGCAGAAGATCCAATGCTCCCAGCCTATATTGTTCTCACC
GAAAGTGCCTGCGTATATTCATCCAAGGAAGTATCTCGTGGAAACACCACCGGTAGA
CGAGACTCCGGAGCCATCGGCAGAGAACCAATCCACAGAGGGGACACCTGAACAA
CCACCACTTATAACCGAGGATGAGACCAGGACTAGAACGCCTGAGCCGATCATCAT
CGAAGAGGAAGAAGAGGATAGCATAAGTTTGCTGTCAGATGGCCCGACCCACCAGG
TGCTGCAAGTCGAGGCAGACATTCACGGGCCGCCCTCTGTATCTAGCTCATCCTGG
TCCATTCCTCATGCATCCGACTTTGATGTGGACAGTTTATCCATACTTGACACCCTGG
AGGGAGCTAGCGTGACCAGCGGGGCAACGTCAGCCGAGACTAACTCTTACTTCGCA
AAGAGTATGGAGTTTCTGGCGCGACCGGTGCCTGCGCCTCGAACAGTATTCAGGAA
CCCTCCACATCCCGCTCCGCGCACAAGAACACCGTCACTTGCACCCAGCAGGGCCT
GCTCGAGAACCAGCCTAGTTTCCACCCCGCCAGGCGTGAATAGGGTGATCACTAGA
GAGGAGCTCGAGGCGCTTACCCCGTCACGCACTCCTAGCAGGTCGGTCTCGAGAA
CCAGCCTGGTCTCCAACCCGCCAGGCGTAAATAGGGTGATTACAAGAGAGGAGTTT
GAGGCGTTCGTAGCACAACAACAATGACGGTTTGATGCGGGTGCATACATCTTTTCC
TCCGACACCGGTCAAGGGCATTTACAACAAAAATCAGTAAGGCAAACGGTGCTATCC
GAAGTGGTGTTGGAGAGGACCGAATTGGAGATTTCGTATGCCCCGCGCCTCGACCA
AGAAAAAGAAGAATTACTACGCAAGAAATTACAGTTAAATCCCACACCTGCTAACAGA
AGCAGATACCAGTCCAGGAAGGTGGAGAACATGAAAGCCATAACAGCTAGACGTAT
TCTGCAAGGCCTAGGGCATTATTTGAAGGCAGAAGGAAAAGTGGAGTGCTACCGAA
CCCTGCATCCTGTTCCTTTGTATTCATCTAGTGTGAACCGTGCCTTTTCAAGCCCCAA
GGTCGCAGTGGAAGCCTGTAACGCCATGTTGAAAGAGAACTTTCCGACTGTGGCTT
CTTACTGTATTATTCCAGAGTACGATGCCTATTTGGACATGGTTGACGGAGCTTCATG
CTGCTTAGACACTGCCAGTTTTTGCCCTGCAAAGCTGCGCAGCTTTCCAAAGAAACA
CTCCTATTTGGAACCCACAATACGATCGGCAGTGCCTTCAGCGATCCAGAACACGCT
CCAGAACGTCCTGGCAGCTGCCACAAAAAGAAATTGCAATGTCACGCAAATGAGAG
AATTGCCCGTATTGGATTCGGCGGCCTTTAATGTGGAATGCTTCAAGAAATATGCGT
GTAATAATGAATATTGGGAAACGTTTAAAGAAAACCCCATCAGGCTTACTGAAGAAAA
CGTGGTAAATTACATTACCAAATTAAAAGGACCAAAAGCTGCTGCTCTTTTTGCGAAG
ACACATAATTTGAATATGTTGCAGGACATACCAATGGACAGGTTTGTAATGGACTTAA
AGAGAGACGTGAAAGTGACTCCAGGAACAAAACATACTGAAGAACGGCCCAAGGTA
CAGGTGATCCAGGCTGCCGATCCGCTAGCAACAGCGTATCTGTGCGGAATCCACCG
AGAGCTGGTTAGGAGATTAAATGCGGTCCTGCTTCCGAACATTCATACACTGTTTGA
TATGTCGGCTGAAGACTTTGACGCTATTATAGCCGAGCACTTCCAGCCTGGGGATTG
TGTTCTGGAAACTGACATCGCGTCGTTTGATAAAAGTGAGGACGACGCCATGGCTCT
GACCGCGTTAATGATTCTGGAAGACTTAGGTGTGGACGCAGAGCTGTTGACGCTGA
TTGAGGCGGCTTTCGGCGAAATTTCATCAATACATTTGCCCACTAAAACTAAATTTAA
ATTCGGAGCCATGATGAAATCTGGAATGTTCCTCACACTGTTTGTGAACACAGTCATT
AACATTGTAATCGCAAGCAGAGTGTTGAGAGAACGGCTAACCGGATCACCATGTGCA
GCATTCATTGGAGATGACAATATCGTGAAAGGAGTCAAATCGGACAAATTAATGGCA
GACAGGTGCGCCACCTGGTTGAATATGGAAGTCAAGATTATAGATGCTGTGGTGGG
CGAGAAAGCGCCTTATTTCTGTGGAGGGTTTATTTTGTGTGACTCCGTGACCGGCAC
AGCGTGCCGTGTGGCAGACCCCCTAAAAAGGCTGTTTAAGCTTGGCAAACCTCTGG
CAGCAGACGATGAACATGATGATGACAGGAGAAGGGCATTGCATGAAGAGTCAACA
CGCTGGAACCGAGTGGGTATTCTTTCAGAGCTGTGCAAGGCAGTAGAATCAAGGTA
TGAAACCGTAGGAACTTCCATCATAGTTATGGCCATGACTACTCTAGCTAGCAGTGT
TAAATCATTCAGCTACCTGAGAGGGGCCCCTATAACTCTCTACGGCTAACCTGAATG
GACTACGACATAGTCTAGTCGACGCCACCAAGTCTAGACGGCGCGCCCACCCAGCG
GCCGCATACAGCAGCAATTGGCAAGCTGCTTACATAGAACTCGCGGCGATTGGCAT
GCCGCCTTAAAATTTTTATTTTATTTTTCTTTTCTTTTCCGAATCGGATTTTGTTTTTAA
TATTTCAAAAAAAAAAAAAAAAAAAAAAAAAAAAAAAAAAAAAAAAGAAGAGCGTTTAA
ACACGTGATATCTGGCCTCATGGGCCTTCCTTTCACTGCCCGCTTTCCAGTCGGGAA
ACCTGTCGTGCCAGCTGCATTAACATGGTCATAGCTGTTTCCTTGCGTATTGGGCGC
TCTCCGCTTCCTCGCTCACTGACTCGCTGCGCTCGGTCGTTCGGGTAAAGCCTGGG
GTGCCTAATGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAATACA
CGGTGCCTGACTGCGTTAGCAATTTAACTGTGATAAACTACCGCATTAAAGCTTATC
GATGATAAGCTGTCAAACATGAGAATTCTTAGAAAAACTCATCGAGCATCAAATGAAA
CTGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGG
TCTGCGATTCCGACTCGTCCAACATCAATACAACCTATTAATTTCCCCTCGTCAAAAA
TAAGGTTATCAAGTGAGAAATCACCATGAGTGACGACTGAATCCGGTGAGAATGGCA
AAAGCTTATGCATTTCTTTCCAGACTTGTTCAACAGGCCAGCCATTACGCTCGTCATC
AAAATCACTCGCATCAACCAAACCGTTATTCATTCGTGATTGCGCCTGAGCGAGACG
AAATACGCGATCGCTGTTAAAAGGACAATTACAAACAGGAATCGAATGCAACCGGCG
CAGGAACACTGCCAGCGCATCAACAATATTTTCACCTGAATCAGGATATTCTTCTAAT
ACCTGGAATGCTGTTTTCCCGGGGATCGCAGTGGTGAGTAACCATGCATCATCAGG
AGTACGGATAAAATGCTTGATGGTCGGAAGAGGCATAAATTCCGTCAGCCAGTTTAG
TCTGACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCCCG
ACATTATCGCGAGCCCATTTATACCCATATAAATCAGCATCCATGTTGGAATTTAATC
GCGGCCTCGAGCAAGACGTTTCCCGTTGAATATGGCTCATAACACCCCTTGTATTAC
TGTTTATGTAAGCAGACAGTTTTATTGTTCATGAGCGGATACATATTTGAATGTATTTA
GAAAAATAAACAAATAGGGGTTCCGCGCACATTTCCCCGAAAAGTGCCACCTAAATT
GTAAGCGTTAATATTTTGTTAAAATTCGCGTTAAATTTTTGTTAAATCAGCTCATTTTTT
AACCAATAGGCCGAAATCGGCAAAATCCCTTATAAATCAAAAGAATAGACCGAGATA
GGGTTGAGTGGCCGCTACAGGGCGCTCCCATTCGCCATTCAGGCTGCGCAACTGTT
GGGAAGGGCGTTTCGGTGCGGGCCTCTTCGCTATTACGCCAGCTGGCGAAAGGGG
GATGTGCTGCAAGGCGATTAAGTTGGGTAACGCCAGGGTTTTCCCAGTCACACGCG
TAATACGACTCACTATAG

The PNI V101 vector map is shown in FIG. 1. The T7 RNA polymerase-based in vitro transcription employed here has high enzymatic processivity, which leads to the circular plasmids generating long heterogeneous RNA transcripts in higher quantities. Therefore, the complete linearization of the circular plasmid is useful to ensure the efficient synthesis of transcripts of defined length. In addition, the restriction enzyme used for this purpose must be carefully chosen, considering that only a single double-stranded cut must be made in the entire length of the plasmid. It is possible to incorporate various types of restriction endonuclease cut sequences including SapI, BspQI, PmlI, EcoRV or Pmel, closer to or after the Poly A tail sequence, encoded into the PNI V101 vector design located at 7,749-7,767 bp. This allows for a single double-stranded DNA cut, thereby generating the linearized DNA template, which terminates at or after Poly A tail. Unique sequences corresponding to the above-mentioned restriction endonuclease cut sites which are incorporated into the vector are listed in Table A.

TABLE A

| Unique Restriction | |
|---|---|
| endonucleases | Example sequences |
| PmeI | 5′-GTTTAAAC-3′ |
| | 3′-CAAATTTG-5′ |
| | or |
| | 5′-CAAATTTG-3′ |
| | 3′-GTTTAAAC-5′ |
| BspQI | 5′-AAAAGAAGAGC-3′ |
| | (SEQ ID NO: 15) |
| | 3′-TTTTCTTCTCG-5′ |
| | (SEQ ID NO: 16) |
| | or |
| | 5′-GCTCTTCTTTT-3′ |
| | (SEQ ID NO: 17) |
| | 3′-CGAGAAGAAAA-5′ |
| | (SEQ ID NO: 18) |
| PmlI | 5′-CACGTG-3′ |
| | 3′-GTGCAC-5′ |
| | Or |
| | 5′-CACGTG-3′ |
| | 3′-GTGCAC-5′ |
| EcoRV | 5′-GATATC-3′ |
| | 3′-CTATAG-5′ |
| | Or |
| | 5′-GATATC-3′ |
| | 3′-CTATAG-5′ |

The restriction endonuclease sequence orientation, whether it is 5′>3′ or 3′>5′, may allow for a closer cut to the Poly A tail than may be necessary for the negative strand synthesis of the self-amplifying RNA and subsequent mRNA expression to generate the protein of interest from the PNI V101 vector encoding the specific gene of interest.

FIG. 2 is a schematic showing linearization of the PNI V101 vector with an insertion of a 3822 bp gene sequence encoding for SARS-COV-2 spike protein. PmeI restriction enzyme linearization (shown at the top of the figure) generates an 11-nucleotide blunt-end overhang after the poly (A) tail. BspQI restriction enzyme (shown at the bottom of the figure) linearizes at the end of the poly (A) tail, producing a staggered end of 3 thymine nucleotides. The presence of 3 thymine nucleotides appears to promote better mRNA expression compared to the presence of the 11-nucleotide staggered blunt end in the final self-amplifying mRNA.

### Antigen Design

### PNI A1

PNI A1 is based on full-length SARS COV 2 surface glycoprotein S from the NCBI database reference MN908947.3, which was codon-optimized for expression in humans. The original sequence (SEQ ID NO: 2 and Table 2) and codon optimized sequence (SEQ ID NO: 3 and Table 3) are shown below.

TABLE 2
SEQ ID NO: 2
Original nucleotide sequence encoding SARS-CoV-2 Spike protein
sequence MN908947.3

ATGTTTGTTTTTCTTGTTTTATTGCCACTAGTCTCTAGTCAGTGTGTTAATCTT
ACAACCAGAACTCAATTACCCCCTGCATACACTAATTCTTTCACACGTGGTGT
TTATTACCCTGACAAAGTTTTCAGATCCTCAGTTTTACATTCAACTCAGGACTT
GTTCTTACCTTTCTTTTCCAATGTTACTTGGTTCCATGCTATACATGTCTCTGG
GACCAATGGTACTAAGAGGTTTGATAACCCTGTCCTACCATTTAATGATGGTG
TTTATTTTGCTTCCACTGAGAAGTCTAACATAATAAGAGGCTGGATTTTTGGT
ACTACTTTAGATTCGAAGACCCAGTCCCTACTTATTGTTAATAACGCTACTAAT
GTTGTTATTAAAGTCTGTGAATTTCAATTTTGTAATGATCCATTTTTGGGTGTT
TATTACCACAAAAACAACAAAAGTTGGATGGAAAGTGAGTTCAGAGTTTATTC
TAGTGCGAATAATTGCACTTTTGAATATGTCTCTCAGCCTTTTCTTATGGACCT
TGAAGGAAAACAGGGTAATTTCAAAAATCTTAGGGAATTTGTGTTTAAGAATA
TTGATGGTTATTTTAAAATATATTCTAAGCACACGCCTATTAATTTAGTGCGTG
ATCTCCCTCAGGGTTTTTCGGCTTTAGAACCATTGGTAGATTTGCCAATAGGT
ATTAACATCACTAGGTTTCAAACTTTACTTGCTTTACATAGAAGTTATTTGACT
CCTGGTGATTCTTCTTCAGGTTGGACAGCTGGTGCTGCAGCTTATTATGTGG
GTTATCTTCAACCTAGGACTTTTCTATTAAAATATAATGAAAATGGAACCATTA
CAGATGCTGTAGACTGTGCACTTGACCCTCTCTCAGAAACAAAGTGTACGTT
GAAATCCTTCACTGTAGAAAAAGGAATCTATCAAACTTCTAACTTTAGAGTCC
AACCAACAGAATCTATTGTTAGATTTCCTAATATTACAAACTTGTGCCCTTTTG
GTGAAGTTTTTAACGCCACCAGATTTGCATCTGTTTATGCTTGGAACAGGAAG
AGAATCAGCAACTGTGTTGCTGATTATTCTGTCCTATATAATTCCGCATCATTT
TCCACTTTTAAGTGTTATGGAGTGTCTCCTACTAAATTAAATGATCTCTGCTTT
ACTAATGTCTATGCAGATTCATTTGTAATTAGAGGTGATGAAGTCAGACAAAT
CGCTCCAGGGCAAACTGGAAAGATTGCTGATTATAATTATAAATTACCAGATG
ATTTTACAGGCTGCGTTATAGCTTGGAATTCTAACAATCTTGATTCTAAGGTT
GGTGGTAATTATAATTACCTGTATAGATTGTTTAGGAAGTCTAATCTCAAACCT
TTTGAGAGAGATATTTCAACTGAAATCTATCAGGCCGGTAGCACACCTTGTAA
TGGTGTTGAAGGTTTTAATTGTTACTTTCCTTTACAATCATATGGTTTCCAACC
CACTAATGGTGTTGGTTACCAACCATACAGAGTAGTAGTACTTTCTTTTGAAC
TTCTACATGCACCAGCAACTGTTTGTGGACCTAAAAAGTCTACTAATTTGGTT
AAAAACAAATGTGTCAATTTCAACTTCAATGGTTTAACAGGCACAGGTGTTCT
TACTGAGTCTAACAAAAAGTTTCTGCCTTTCCAACAATTTGGCAGAGACATTG
CTGACACTACTGATGCTGTCCGTGATCCACAGACACTTGAGATTCTTGACATT
ACACCATGTTCTTTTGGTGGTGTCAGTGTTATAACACCAGGAACAAATACTTC
TAACCAGGTTGCTGTTCTTTATCAGGATGTTAACTGCACAGAAGTCCCTGTTG
CTATTCATGCAGATCAACTTACTCCTACTTGGCGTGTTTATTCTACAGGTTCT
AATGTTTTTCAAACACGTGCAGGCTGTTTAATAGGGGCTGAACATGTCAACAA
CTCATATGAGTGTGACATACCCATTGGTGCAGGTATATGCGCTAGTTATCAG
ACTCAGACTAATTCTCCTCGGCGGGCACGTAGTGTAGCTAGTCAATCCATCA
TTGCCTACACTATGTCACTTGGTGCAGAAAATTCAGTTGCTTACTCTAATAAC
TCTATTGCCATACCCACAAATTTTACTATTAGTGTTACCACAGAAATTCTACCA
GTGTCTATGACCAAGACATCAGTAGATTGTACAATGTACATTTGTGGTGATTC
AACTGAATGCAGCAATCTTTTGTTGCAATATGGCAGTTTTTGTACACAATTAAA
CCGTGCTTTAACTGGAATAGCTGTTGAACAAGACAAAAACACCCAAGAAGTTT
TTGCACAAGTCAAACAAATTTACAAAACACCACCAATTAAAGATTTTGGTGGT
TTTAATTTTTCACAAATATTACCAGATCCATCAAAACCAAGCAAGAGGTCATTT
ATTGAAGATCTACTTTTCAACAAAGTGACACTTGCAGATGCTGGCTTCATCAA
ACAATATGGTGATTGCCTTGGTGATATTGCTGCTAGAGACCTCATTTGTGCAC
AAAAGTTTAACGGCCTTACTGTTTTGCCACCTTTGCTCACAGATGAAATGATT
GCTCAATACACTTCTGCACTGTTAGCGGGTACAATCACTTCTGGTTGGACCTT
TGGTGCAGGTGCTGCATTACAAATACCATTTGCTATGCAAATGGCTTATAGGT
TTAATGGTATTGGAGTTACACAGAATGTTCTCTATGAGAACCAAAAATTGATT
GCCAACCAATTTAATAGTGCTATTGGCAAAATTCAAGACTCACTTTCTTCCAC
AGCAAGTGCACTTGGAAAACTTCAAGATGTGGTCAACCAAAATGCACAAGCT
TTAAACACGCTTGTTAAACAACTTAGCTCCAATTTTGGTGCAATTTCAAGTGTT
TTAAATGATATCCTTTCACGTCTTGACAAAGTTGAGGCTGAAGTGCAAATTGA
TAGGTTGATCACAGGCAGACTTCAAAGTTTGCAGACATATGTGACTCAACAAT
TAATTAGAGCTGCAGAAATCAGAGCTTCTGCTAATCTTGCTGCTACTAAAATG
TCAGAGTGTGTACTTGGACAATCAAAAAGAGTTGATTTTTGTGGAAAGGGCTA
TCATCTTATGTCCTTCCCTCAGTCAGCACCTCATGGTGTAGTCTTCTTGCATG
TGACTTATGTCCCTGCACAAGAAAAGAACTTCACAACTGCTCCTGCCATTTGT
CATGATGGAAAAGCACACTTTCCTCGTGAAGGTGTCTTTGTTTCAAATGGCAC
ACACTGGTTTGTAACACAAAGGAATTTTTATGAACCACAAATCATTACTACAG
ACAACACATTTGTGTCTGGTAACTGTGATGTTGTAATAGGAATTGTCAACAAC
ACAGTTTATGATCCTTTGCAACCTGAATTAGACTCATTCAAGGAGGAGTTAGA
TAAATATTTTAAGAATCATACATCACCAGATGTTGATTTAGGTGACATCTCTGG
CATTAATGCTTCAGTTGTAAACATTCAAAAAGAAATTGACCGCCTCAATGAGG
TTGCCAAGAATTTAAATGAATCTCTCATCGATCTCCAAGAACTTGGAAAGTAT
GAGCAGTATATAAAATGGCCATGGTACATTTGGCTAGGTTTTATAGCTGGCTT
GATTGCCATAGTAATGGTGACAATTATGCTTTGCTGTATGACCAGTTGCTGTA
GTTGTCTCAAGGGCTGTTGTTCTTGTGGATCCTGCTGCAAATTTGATGAAGA
CGACTCTGAGCCAGTGCTCAAAGGAGTCAAATTACATTACACATAA

TABLE 3
SEQ ID. No. 3
PNI A1 Codon optimized nucleotide sequence encoding the SARS-
CoV-2 Spike protein sequence

ATGTTCGTGTTCCTGGTGCTGCTGCCCCTGGTGTCCTCACAGTGCGTGAATC
TGACCACTCGTACTCAGCTGCCCCCAGCATACACCAATTCTTTCACCAGAGG
GGTCTACTACCCCGACAAAGTGTTCAGGAGTTCCGTCTTGCATTCCACACAG
GACCTGTTCCTGCCATTCTTCAGCAATGTCACCTGGTTCCACGCGATCCACG
TTTCTGGCACCAACGGGACAAAGCGGTTCGACAACCCTGTTCTGCCGTTCAA
CGATGGCGTGTACTTTGCGTCCACCGAGAAAAGCAACATCATCCGCGGCTG
GATCTTCGGGACTACCCTGGACTCGAAGACTCAATCGCTGCTGATCGTCAAC
AACGCAACGAACGTGGTGATCAAGGTGTGCGAATTCCAGTTCTGCAACGACC
CGTTCCTTGGCGTCTACTACCACAAGAACAACAAGAGTTGGATGGAGTCCGA
GTTCCGCGTTTACTCTTCCGCCAACAACTGCACATTCGAGTACGTGTCGCAA
CCTTTCCTGATGGACCTGGAGGGCAAGCAGGGCAACAAAAAGAACCTGCGC
GAGTTCGTCTTCAAGAACATTGTCGGCTACTTCAAGATTTACTCCAAGCACAC
CCCGATCAACCTTGTGCGCGATCTACCTCAGGGATTCTCTGCTCTGGAGCCA
CTGGTGGACCTGCCCATTGGGATCAATATAACGCGTTTCCAGACCCTGCTGG
CTTTACACCGCTCCTATTTGACCCCCGCCGATTCTTCCTCCGGCTGGACGGC
CGGCGCCGCAGCCTATTACGTAGGCTACCTGCAGCCGCGCACCTTCCTTTT
AAAGTACAACGAGAACGGCACTATCACCGACGCAGTAGATTGTGCTCTGGAC
CCTCTGAGCGAGACCAAATGCACTCTGAAGTCTTTTACAGTGGAGAAGGGAA
TCTATCAGGGTGCCAACTTCCGCGTGCAGCCCACCGAGAGCATCGACAGGT
TCCCCAACATCACGAATCTGTGCCCATTCGGCGAGGTGTTCAACGCCACCC
GCTTCGCATCCGTGTACGCGTGGAACCGGAAGCGCATCTCCAATTGCGTGG
CGGACTACTCTGTTCTCGTAAACTCCGCTTCGTTCTCCACGTTCAAGTGCTAT
GGCGTCTCCCCTACCAAACTAAACGACCTGTGCTTTACCAACGTATACGCGG
ACTCGTTTGTGATTCGCGGCGTCGAGGTGCGCCAGATCGCTCCGGGCCAGA
CTGGCAAGATTGCAGACTACAACTACAAGCTGCCCGACGACTTCACCGGCT
GCGTGATCGCGTGGGTTGGAAACAACCTGGACAGCAAGGTGGGGGGGAAC
GTAAACTACCTGTACCGTCTGTTCCGCAAGATGAACCTGAGGCCGGGAGAG
CGCGATATTTCTACCGAGATTTACCAGGCCGGCTCCACCCCTTGCAACGGC
GTGGAGGGCTTCAACTGCTACTTCCCCCTGCAGAGCTACGGCTTCCAGCCG
ACGATCGGTGTCGGCTATCAGCCATACCGGGTGGTGGTGTTGAGTTTCGAG
CTCCTGCACGCCCCCGCCACTGTCTGTGGACCCAAGAAGTCCACCAACCTTT
ACAAGAACAAGTGCGTGAACTTCAACTTCAACGGGCTGACCGGCACGGGTG
TGCTGACCGAAAGCAACAAGAAATTTCTCCCCTTCCAGCAGTTTGGACGAGA
CATCGCGGACACGACCGACGCTGTGAGGGACCCTCAGACCCTGGAGATCCT
GGACATCACTCCTTGCTCCTTTGGTGGCGTGTCCGTGATCACGCCCGGTAC
CAACCGAAGCAACCAGGTCGCCGTGCTGTACCAAGACGTGAACTGCACCGA
GGTGCCCGTGGCGATCCACGCCGACCAGTTGACCCCAACCTGGCGAGTGTA
CTCGACCGGGTCCAACGTGTTCCAGACTCGCGCCGGCTGCTTGATCGGTGC
CGAGCACGTCAACAACAGCTACGAGTGCGACATCCCCATCGGTGCGGGTAT
TTGTGCCTCCTACCAGACCCAGACCAACTCCCCGCGGCGTGCGCGCAGTGT
GGCTTCCCAGAGCATCATCGCCTACACCATGTCTCTGGGTGCGGAGAATTCT
GTCGCCACGAGCAACAATTCGATCGCTATCCCCACCAACTTCACCATCAGCG
TCACCACCGAAATCCTCCCCGTGTCCATGACCAAGACCTCGGTCGATTGCAC
TATGTATATTTGCGGGGACTCAACCGAATGCTCCAACCTTCTGCTCCAGTAC
GGCTCTTTTTGTACGCAACTCAACCGAGCTTTGACTGGGATCGCGGTCGAGC
AGGACAAAAATACCCAGGAGGTGTTCGCGCAGGTGAAGCAGATATACAAGA
CCCCCCCCATCAAGGATTTCGGCGGCTTCAACTTCTCACAGATCCTACCCGA
CCCGTCCAAACCCTCGAAGCGCAGCTTCATCGAGGACCTGCTGTTCAACAAA
GTGACACTGGCCGACGCTGGATTTATCAAGCAATACGGCGATTGCCTGGGC
GACATCGCGGCCCGCGACCTGATCTGCGCCCAGAAATTCAACGGGCTAACC
GTGCTTCCCCCGCTGCTGACCGACGAGATGATCGCCCAGTACACGTCGGCC
CTTCTGGCTGGCACGATCACCAGCGGCTGGACCTTCGGCGCTGGCGCCGC
GCTCCAGATCCCCTTTGCCATGCAGATGGCCTACAGATTCAACGGCATCGGC
GTGACACAGAATGTGCTGTACGAGAACCAGAAGTTGATCGCCAATCAGTTCA
ACAGCGCGATAGGCAAGATCCAGGATTCATTGTCATCCACCGCAAGCGCCC
TGGGCAAGCTGCAGGACGTGGTCAACCAGAACGCTCAGGCTCTCAACACAC
TGGTGCCTCAGTTGTCCAGCAACTTTGGTGCCATCTCCTCCCCACTCAACGA
CATCCTTTCGCGTCTGGACAAAGTCGAGGCCGAGGTGCAGATCGACAGGCT
GATCACCGGACGCTTGCAGAGCTTGCAGACTTACGTGACCCAACAGCTGATA
CGCGCTGCAGAAATCCGGGCGTCCGCCAACCTGGCAGCGACCAAGATGTCC
GAGTGCGTGCTGGGACAGTCCAAGCGAGACGACTTCTGTGGAAAAGGCTAC
CACCTGATGTCCTTTCCTCAGTCCGCCCCCCATGGAGTGGTGTTTCTGCACG
TCACCTACGTGCCCGCCCAAGAGAAGAACTTCACCACTGCGCCTGCGATCT
GTCACGACGGGAAGGCTCACTTTCCGCGCGAGGGGGTGTTCGTCTCCAACG
GCACCCACTGGTTCGTGACTCAGCGCAACTTTTACGAGCCCCAGATCATCAC
CACCGACAACACTTTCGTATCCGGCAATTGCGATGTGGTAATTGGTATTGTG
AACAACACCGTGTACGACCCCTTGCAGCCAGAACTCGACTCTTTCAAGGAGG
AGCTCGACAAGTACTTCAAGAACCATACATCGCCAGATGTGGACTTAGGCGA
CATCTCAGGAATCAACGCCTCCGTGGTCAACATCCAGAAGGAGATCGACCG
CCTGAACGAGGTCGCCAAGAACCTAAACGAAAGCCTGATCGACCTGCAGGA
GCTGGGCAAGTACGAGCAGTACATCAAATGGCCGTGGTACATCTGGCTTGG
TTTCATCGCTGGCCTCATTGCCATTGTGATGGTGACCATCATGCTATGCTGTA
TGACCTCCTGCTGTTCTTGCCTGAAGGGCTGTTGTTCTTGCGGCTCCTGTTG
CAAATTCGACGAAGGCGCTTCAGAGCCGGTGTTGAAGGGCGTCAAGCTGCA
CTATACCTGA

Codon changes were made for generating humanized SARS-COV-2 spike protein PNI A1. The codon changes are shown in Table 4.

TABLE 4

| Codon | Nucleotide | Original | Modified |
|---|---|---|---|
| changes | position | base | base |
| F186K | 556-558 | TTC | AAA |
| D198V | 592-594 | GAT | GTC |
| G252A | 754-756 | GGT | GCC |
| T315G | 943-945 | ACT | GGT |
| S316A | 946-948 | TCT | GCC |
| V327D | 979-981 | GTT | GAC |
| Y369V | 1105-1107 | TAT | GTA |
| D405V | 1213-1215 | GAT | GTC |
| N437V | 1309-1311 | AAT | GTT |
| S438G | 1312-1314 | TCT | GGA |
| Y449V | 1345-1347 | TAT | GTA |
| S459M | 1375-1377 | TCT | ATG |
| F464G | 1390-1392 | TTT | GGA |
| N501I | 1501-1503 | AAT | ATC |
| V534Y | 1600-1602 | GTT | TAC |
| T604R | 1810-1812 | ACT | CGA |
| Y707T | 2119-2121 | TAC | ACG |
| K964P | 2890-2892 | AAA | CCT |
| V976P | 2926-2928 | GTT | CCA |
| V1040D | 3118-3120 | GTT | GAC |
| D1259G | 3775-3777 | GAC | GGC |
| D1260A | 3778-3780 | GAC | GCT |

### PNI A2

PNI A2 is based on full-length SARS-COV-2 surface glycoprotein S from the NCBI database reference MN908947.3, which was codon-optimized for expression in humans (as shown above). The following point mutations were made in the codon optimized sequence to encode the prefusion version of Spike protein and target the D614G dominant mutant lineages of SARS-COV-2 virus. The complete codon optimized sequence incorporating the mutations is shown below in Table 5. The key changes to codon sequences are shown below in Table 6.

TABLE 5
SEQ ID NO: 4
PNI A2

ATGTTCGTGTTCCTGGTGCTGCTGCCCCTGGTGAGCAGCCAGTGCGTGAAC
CTGACCACCAGAACCCAGCTGCCCCCCGCCTACACCAACAGCTTCACCAGA
GGCGTGTACTACCCCGACAAGGTGTTCAGAAGCAGCGTGCTGCACAGCACC
CAGGACCTGTTCCTGCCCTTCTTCAGCAACGTGACCTGGTTCCACGCCATCC
ACGTGAGCGGCACCAACGGCACCAAGAGATTCGACAACCCCGTGCTGCCCT
TCAACGACGGCGTGTACTTCGCCAGCACCGAGAAGAGCAACATCATCAGAG
GCTGGATCTTCGGCACCACCCTGGACAGCAAGACCCAGAGCCTGCTGATCG
TGAACAACGCCACCAACGTGGTGATCAAGGTGTGCGAGTTCCAGTTCTGCAA
CGACCCCTTCCTGGGCGTGTACTACCACAAGAACAACAAGAGCTGGATGGA
GCCCGAGTTCAGAGTGTACAGCAGCGCCAACAACTGCACCTTCGAGTACGT
GAGCCAGCCCTTCCTGATGGACCTGGAGGGCAAGCAGGGCAACAAGAAGAA
CCTGAGAGAGTTCGTGTTCAAGAACATCGTGGGCTACTTCAAGATCTACAGC
AAGCACACCCCCATCAACCTGGTGAGAGACCTGCCCCAGGGCTTCAGCGCC
CTGGAGCCCCTGGTGGACCTGCCCATCGGCATCAACATCACCAGATTCCAG
ACCCTGCTGGCCCTGCACAGAAGCTACCTGACCCCCGCCGACAGCAGCAGC
GGCTGGACCGCCGGCGCCGCCGCCTACTACGTGGGCTACCTGCAGCCCAG
AACCTTCCTGCTGAAGTACAACGAGAACGGCACCATCACCGACGCCGTGGA
CTGCGCCCTGGACCCCCTGAGCGAGACCAAGTGCACCCTGAAGAGCTTCAC
CGTGGAGAAGGGCATCTACCAGGGCGCCAACTTCAGAGTGCAGCCCACCGA
GAGCATCGACAGATTCCCCAACATCACCAACCTGTGCCCCTTCGGCGAGGT
GTTCAACGCCACCAGATTCGCCAGCGTGTACGCCTGGAACAGAAAGAGAAT
CAGCAACTGCGTGGCCGACTACAGCGTGCTGGTGAACAGCGCCAGCTTCAG
CACCTTCAAGTGCTACGGCGTGAGCCCCACCAAGCTGAACGACCTGTGCTT
CACCAACGTGTACGCCGACAGCTTCGTGATCAGAGGCGTGGAGGTGAGACA
GATCGCCCCCGGCCAGACCGGCAAGATCGCCGACTACAACTACAAGCTGCC
CGACGACTTCACCGGCTGCGTGATCGCCTGGGTGGGCAACAACCTGGACAG
CAAGGTGGGCGGCAACGTGAACTACCTGTACAGACTGTTCAGAAAGATGAA
CCTGAGACCCGGCGAGAGAGACATCAGCACCGAGATCTACCAGGCCGGCA
GCACCCCCTGCAACGGCGTGGAGGGCTTCAACTGCTACTTCCCCCTGCAGA
GCTACGGCTTCCAGCCCACCATCGGCGTGGGCTACCAGCCCTACAGAGTGG
TGGTGCTGAGCTTCGAGCTGCTGCACGCCCCCGCCACCGTGTGCGGCCCC
AAGAAGAGCACCAACCTGTACAAGAACAAGTGCGTGAACTTCAACTTCAACG
GCCTGACCGGCACCGGCGTGCTGACCGAGAGCAACAAGAAGTTCCTGCCCT
TCCAGCAGTTCGGCAGAGACATCGCCGACACCACCGACGCCGTGAGAGACC
CCCAGACCCTGGAGATCCTGGACATCACCCCCTGCAGCTTCGGCGGCGTGA
GCGTGATCACCCCCGGCACCAACAGAAGCAACCAGGTGGCCGTGCTGTACC
AGGGCGTGAACTGCACCGAGGTGCCCGTGGCCATCCACGCCGACCAGCTG
ACCCCCACCTGGAGAGTGTACAGCACCGGCAGCAACGTGTTCCAGACCAGA
GCCGGCTGCCTGATCGGCGCCGAGCACGTGAACAACAGCTACGAGTGCGA
CATCCCCATCGGCGCCGGCATCTGCGCCAGCTACCAGACCCAGACCAACAG
CCCCAGAAGAGCCAGAAGCGTGGCCAGCCAGAGCATCATCGCCTACACCAT
GAGCCTGGGCGCCGAGAACAGCGTGGCCACCAGCAACAACAGCATCGCCA
TCCCCACCAACTTCACCATCAGCGTGACCACCGAGATCCTGCCCGTGAGCAT
GACCAAGACCAGCGTGGACTGCACCATGTACATCTGCGGCGACAGCACCGA
GTGCAGCAACCTGCTGCTGCAGTACGGCAGCTTCTGCACCCAGCTGAACAG
AGCCCTGACCGGCATCGCCGTGGAGCAGGACAAGAACACCCAGGAGGTGTT
CGCCCAGGTGAAGCAGATCTACAAGACCCCCCCCATCAAGGACTTCGGCGG
CTTCAACTTCAGCCAGATCCTGCCCGACCCCAGCAAGCCCAGCAAGAGAAG
CTTCATCGAGGACCTGCTGTTCAACAAGGTGACCCTGGCCGACGCCGGCTT
CATCAAGCAGTACGGCGACTGCCTGGGCGACATCGCCGCCAGAGACCTGAT
CTGCGCCCAGAAGTTCAACGGCCTGACCGTGCTGCCCCCCCTGCTGACCGA
CGAGATGATCGCCCAGTACACCAGCGCCCTGCTGGCCGGCACCATCACCAG
CGGCTGGACCTTCGGCGCCGGCGCCGCCCTGCAGATCCCCTTCGCCATGC
AGATGGCCTACAGATTCAACGGCATCGGCGTGACCCAGAACGTGCTGTACG
AGAACCAGAAGCTGATCGCCAACCAGTTCAACAGCGCCATCGGCAAGATCC
AGGACAGCCTGAGCAGCACCGCCAGCGCCCTGGGCAAGCTGCAGGACGTG
GTGAACCAGAACGCCCAGGCCCTGAACACCCTGGTGCCCCAGCTGAGCAGC
AACTTCGGCGCCATCAGCAGCCCCCTGAACGACATCCTGAGCAGACTGGAC
CCCCCCGAGGCCGAGGTGCAGATCGACAGACTGATCACCGGCAGACTGCA
GAGCCTGCAGACCTACGTGACCCAGCAGCTGATCAGAGCCGCCGAGATCAG
AGCCAGCGCCAACCTGGCCGCCACCAAGATGAGCGAGTGCGTGCTGGGCC
AGAGCAAGAGAGACGACTTCTGCGGCAAGGGCTACCACCTGATGAGCTTCC
CCCAGAGCGCCCCCCACGGCGTGGTGTTCCTGCACGTGACCTACGTGCCC
GCCCAGGAGAAGAACTTCACCACCGCCCCCGCCATCTGCCACGACGGCAAG
GCCCACTTCCCCAGAGAGGGCGTGTTCGTGAGCAACGGCACCCACTGGTTC
GTGACCCAGAGAAACTTCTACGAGCCCCAGATCATCACCACCGACAACACCT
TCGTGAGCGGCAACTGCGACGTGGTGATCGGCATCGTGAACAACACCGTGT
ACGACCCCCTGCAGCCCGAGCTGGACAGCTTCAAGGAGGAGCTGGACAAGT
ACTTCAAGAACCACACCAGCCCCGACGTGGACCTGGGCGACATCAGCGGCA
TCAACGCCAGCGTGGTGAACATCCAGAAGGAGATCGACAGACTGAACGAGG
TGGCCAAGAACCTGAACGAGAGCCTGATCGACCTGCAGGAGCTGGGCAAGT
ACGAGCAGTACATCAAGTGGCCCTGGTACATCTGGCTGGGCTTCATCGCCG
GCCTGATCGCCATCGTGATGGTGACCATCATGCTGTGCTGCATGACCAGCT
GCTGCAGCTGCCTGAAGGGCTGCTGCAGCTGCGGCAGCTGCTGCAAGTTCG
ACGAGGGCGCCAGCGAGCCCGTGCTGAAGGGCGTGAAGCTGCACTACACC
TGA

Table 6 shows the codon changes that were made for generating humanized prefusion SARS-COV-2 spike protein PNI A2.

TABLE 6

| Codon | Nucleotide | Original | Modified |
|---|---|---|---|
| changes | position | base | base |
| S155P | 469-471 | AGT | CCC |
| F186K | 556-558 | TTC | AAA |
| D198V | 592-594 | GAT | GTC |
| G252A | 754-756 | GGT | GCC |
| T315G | 943-945 | ACT | GGT |
| S316A | 946-948 | TCT | GCC |
| V327D | 979-981 | GTT | GAC |
| Y369V | 1105-1107 | TAT | GTA |
| D405V | 1213-1215 | GAT | GTC |
| N437V | 1309-1311 | AAT | GTT |
| S438G | 1312-1314 | TCT | GGA |
| Y449V | 1345-1347 | TAT | GTA |
| S459M | 1375-1377 | TCT | ATG |
| F464G | 1390-1392 | TTT | GGA |
| N501I | 1501-1503 | AAT | ATC |
| V534Y | 1600-1602 | GTT | TAC |
| T604R | 1810-1812 | ACT | CGA |
| D614G | 1840-1842 | GAT | GGC |
| Y707T | 2119-2121 | TAC | ACG |
| K964P | 2890-2892 | AAA | CCT |
| V976P | 2926-2928 | GTT | CCA |
| V1040D | 3118-3120 | GTT | GAC |
| D1259G | 3775-3777 | GAC | GGC |
| D1260A | 3778-3780 | GAC | GCT |

### PNI A3

PNI A3 is based on full-length SARS COV 2 surface glycoprotein S from the NCBI database reference MN908947.3. The complete codon optimized sequence is shown below in Table 7. The key changes to codon sequences are shown in Table 8.

TABLE 7
SEQ ID NO: 5

ATGTTCGTGTTCCTGGTGCTGCTGCCCCTGGTGTCCTCACAGTGCGTGAATC
TGACCACTCGTACTCAGCTGCCCCCAGCATACACCAATTCTTTCACCAGAGG
GGTCTACTACCCCGACAAAGTGTTCAGGAGTTCCGTCTTGCATTCCACACAG
GACCTGTTCCTGCCATTCTTCAGCAATGTCACCTGGTTCCACGCGATCCACG
TTTCTGGCACCAACGGGACAAAGCGGTTCGACAACCCTGTTCTGCCGTTCAA
CGATGGCGTGTACTTTGCGTCCACCGAGAAAAGCAACATCATCCGCGGCTG
GATCTTCGGGACTACCCTGGACTCGAAGACTCAATCGCTGCTGATCGTCAAC
AACGCAACGAACGTGGTGATCAAGGTGTGCGAATTCCAGTTCTGCAACGACC
CGTTCCTTGGCGTCTACTACCACAAGAACAACAAGAGTTGGATGGAGCCCGA
GTTCCGCGTTTACTCTTCCGCCAACAACTGCACATTCGAGTACGTGTCGCAA
CCTTTCCTGATGGACCTGGAGGGCAAGCAGGGCAACAAAAAGAACCTGCGC
GAGTTCGTCTTCAAGAACATTGTCGGCTACTTCAAGATTTACTCCAAGCACAC
CCCGATCAACCTTGTGCGCGATCTACCTCAGGGATTCTCTGCTCTGGAGCCA
CTGGTGGACCTGCCCATTGGGATCAATATAACGCGTTTCCAGACCCTGCTGG
CTTTACACCGCTCCTATTTGACCCCCGCCGATTCTTCCTCCGGCTGGACGGC
CGGCGCCGCAGCCTATTACGTAGGCTACCTGCAGCCGCGCACCTTCCTTTT
AAAGTACAACGAGAACGGCACTATCACCGACGCAGTAGATTGTGCTCTGGAC
CCTCTGAGCGAGACCAAATGCACTCTGAAGTCTTTTACAGTGGAGAAGGGAA
TCTATCAGGGTGCCAACTTCCGCGTGCAGCCCACCGAGAGCATCGACAGGT
TCCCCAACATCACGAATCTGTGCCCATTCGGCGAGGTGTTCAACGCCACCC
GCTTCGCATCCGTGTACGCGTGGAACCGGAAGCGCATCTCCAATTGCGTGG
CGGACTACTCTGTTCTCGTAAACTCCGCTTCGTTCTCCACGTTCAAGTGCTAT
GGCGTCTCCCCTACCAAACTAAACGACCTGTGCTTTACCAACGTATACGCGG
ACTCGTTTGTGATTCGCGGCGTCGAGGTGCGCCAGATCGCTCCGGGCCAGA
CTGGCAAGATTGCAGACTACAACTACAAGCTGCCCGACGACTTCACCGGCT
GCGTGATCGCGTGGGTTGGAAACAACCTGGACAGCAAGGTGGGGGGGAAC
GTAAACTACCTGTACCGTCTGTTCCGCAAGATGAACCTGAGGCCGGGAGAG
CGCGATATTTCTACCGAGATTTACCAGGCCGGCTCCACCCCTTGCAACGGC
GTGGAGGGCTTCAACTGCTACTTCCCCCTGCAGAGCTACGGCTTCCAGCCG
ACGATCGGTGTCGGCTATCAGCCATACCGGGTGGTGGTGTTGAGTTTCGAG
CTCCTGCACGCCCCCGCCACTGTCTGTGGACCCAAGAAGTCCACCAACCTTT
ACAAGAACAAGTGCGTGAACTTCAACTTCAACGGGCTGACCGGCACGGGTG
TGCTGACCGAAAGCAACAAGAAATTTCTCCCCTTCCAGCAGTTTGGACGAGA
CATCGCGGACACGACCGACGCTGTGAGGGACCCTCAGACCCTGGAGATCCT
GGACATCACTCCTTGCTCCTTTGGTGGCGTGTCCGTGATCACGCCCGGTAC
CAACCGAAGCAACCAGGTCGCCGTGCTGTACCAAGACGTGAACTGCACCGA
GGTGCCCGTGGCGATCCACGCCGACCAGTTGACCCCAACCTGGCGAGTGTA
CTCGACCGGGTCCAACGTGTTCCAGACTCGCGCCGGCTGCTTGATCGGTGC
CGAGCACGTCAACAACAGCTACGAGTGCGACATCCCCATCGGTGCGGGTAT
TTGTGCCTCCTACCAGACCCAGACCAACTCCCCGCGGCGTGCGCGCAGTGT
GGCTTCCCAGAGCATCATCGCCTACACCATGTCTCTGGGTGCGGAGAATTCT
GTCGCCACGAGCAACAATTCGATCGCTATCCCCACCAACTTCACCATCAGCG
TCACCACCGAAATCCTCCCCGTGTCCATGACCAAGACCTCGGTCGATTGCAC
TATGTATATTTGCGGGGACTCAACCGAATGCTCCAACCTTCTGCTCCAGTAC
GGCTCTTTTTGTACGCAACTCAACCGAGCTTTGACTGGGATCGCGGTCGAGC
AGGACAAAAATACCCAGGAGGTGTTCGCGCAGGTGAAGCAGATATACAAGA
CCCCCCCCATCAAGGATTTCGGCGGCTTCAACTTCTCACAGATCCTACCCGA
CCCGTCCAAACCCTCGAAGCGCAGCTTCATCGAGGACCTGCTGTTCAACAAA
GTGACACTGGCCGACGCTGGATTTATCAAGCAATACGGCGATTGCCTGGGC
GACATCGCGGCCCGCGACCTGATCTGCGCCCAGAAATTCAACGGGCTAACC
GTGCTTCCCCCGCTGCTGACCGACGAGATGATCGCCCAGTACACGTCGGCC
CTTCTGGCTGGCACGATCACCAGCGGCTGGACCTTCGGCGCTGGCGCCGC
GCTCCAGATCCCCTTTGCCATGCAGATGGCCTACAGATTCAACGGCATCGGC
GTGACACAGAATGTGCTGTACGAGAACCAGAAGTTGATCGCCAATCAGTTCA
ACAGCGCGATAGGCAAGATCCAGGATTCATTGTCATCCACCGCAAGCGCCC
TGGGCAAGCTGCAGGACGTGGTCAACCAGAACGCTCAGGCTCTCAACACAC
TGGTGCCTCAGTTGTCCAGCAACTTTGGTGCCATCTCCTCCCCACTCAACGA
CATCCTTTCGCGTCTGGACCCCCCCGAGGCCGAGGTGCAGATCGACAGGCT
GATCACCGGACGCTTGCAGAGCTTGCAGACTTACGTGACCCAACAGCTGATA
CGCGCTGCAGAAATCCGGGCGTCCGCCAACCTGGCAGCGACCAAGATGTCC
GAGTGCGTGCTGGGACAGTCCAAGCGAGACGACTTCTGTGGAAAAGGCTAC
CACCTGATGTCCTTTCCTCAGTCCGCCCCCCATGGAGTGGTGTTTCTGCACG
TCACCTACGTGCCCGCCCAAGAGAAGAACTTCACCACTGCGCCTGCGATCT
GTCACGACGGGAAGGCTCACTTTCCGCGCGAGGGGGTGTTCGTCTCCAACG
GCACCCACTGGTTCGTGACTCAGCGCAACTTTTACGAGCCCCAGATCATCAC
CACCGACAACACTTTCGTATCCGGCAATTGCGATGTGGTAATTGGTATTGTG
AACAACACCGTGTACGACCCCTTGCAGCCAGAACTCGACTCTTTCAAGGAGG
AGCTCGACAAGTACTTCAAGAACCATACATCGCCAGATGTGGACTTAGGCGA
CATCTCAGGAATCAACGCCTCCGTGGTCAACATCCAGAAGGAGATCGACCG
CCTGAACGAGGTCGCCAAGAACCTAAACGAAAGCCTGATCGACCTGCAGGA
GCTGGGCAAGTACGAGCAGTACATCAAATGGCCGTGGTACATCTGGCTTGG
TTTCATCGCTGGCCTCATTGCCATTGTGATGGTGACCATCATGCTATGCTGTA
TGACCTCCTGCTGTTCTTGCCTGAAGGGCTGTTGTTCTTGCGGCTCCTGTTG
CAAATTCGACGAAGGCGCTTCAGAGCCGGTGTTGAAGGGCGTCAAGCTGCA
CTATACCTGA

Table 8 shows the codon optimizations made in PNI A3.

TABLE 8

| Codon | Nucleotide | Original | Modified |
|---|---|---|---|
| changes | position | base | base |
| S155P | 469-471 | AGT | CCC |
| F186K | 556-558 | TTC | AAA |
| D198V | 592-594 | GAT | GTC |
| G252A | 754-756 | GGT | GCC |
| T315G | 943-945 | ACT | GGT |
| S316A | 946-948 | TCT | GCC |
| V327D | 979-981 | GTT | GAC |
| Y369V | 1105-1107 | TAT | GTA |
| D405V | 1213-1215 | GAT | GTC |
| N437V | 1309-1311 | AAT | GTT |
| S438G | 1312-1314 | TCT | GGA |
| Y449V | 1345-1347 | TAT | GTA |
| S459M | 1375-1377 | TCT | ATG |
| F464G | 1390-1392 | TTT | GGA |
| N501I | 1501-1503 | AAT | ATC |
| V534Y | 1600-1602 | GTT | TAC |
| T604R | 1810-1812 | ACT | CGA |
| D614G | 1840-1842 | GAT | GGC |
| Y707T | 2119-2121 | TAC | ACG |
| K964P | 2890-2892 | AAA | CCT |
| V976P | 2926-2928 | GTT | CCA |
| V1040D | 3118-3120 | GTT | GAC |
| D1259G | 3775-3777 | GAC | GGC |
| D1260A | 3778-3780 | GAC | GCT |
| K986P | 2955-2957 | CAA | CCC |
| V987P | 2958-2960 | AGT | CCC |

### PNI A4

PNI A4 is based on full-length SARS COV 2 surface glycoprotein S from the NCBI database reference MN908947.3 and codon optimized for mammalian cell expression with the D614G dominant mutation incorporated. The full codon optimized sequence is provided below in Table 9.

TABLE 9
SEQ ID NO: 6

ATGTTCGTGTTCCTGGTGCTGCTGCCCCTGGTGAGCAGCCAGTGCGTGAAC
CTGACCACCAGAACCCAGCTGCCCCCCGCCTACACCAACAGCTTCACCAGA
GGCGTGTACTACCCCGACAAGGTGTTCAGAAGCAGCGTGCTGCACAGCACC
CAGGACCTGTTCCTGCCCTTCTTCAGCAACGTGACCTGGTTCCACGCCATCC
ACGTGAGCGGCACCAACGGCACCAAGAGATTCGACAACCCCGTGCTGCCCT
TCAACGACGGCGTGTACTTCGCCAGCACCGAGAAGAGCAACATCATCAGAG
GCTGGATCTTCGGCACCACCCTGGACAGCAAGACCCAGAGCCTGCTGATCG
TGAACAACGCCACCAACGTGGTGATCAAGGTGTGCGAGTTCCAGTTCTGCAA
CGACCCCTTCCTGGGCGTGTACTACCACAAGAACAACAAGAGCTGGATGGA
GAGCGAGTTCAGAGTGTACAGCAGCGCCAACAACTGCACCTTCGAGTACGT
GAGCCAGCCCTTCCTGATGGACCTGGAGGGCAAGCAGGGCAACTTCAAGAA
CCTGAGAGAGTTCGTGTTCAAGAACATCGACGGCTACTTCAAGATCTACAGC
AAGCACACCCCCATCAACCTGGTGAGAGACCTGCCCCAGGGCTTCAGCGCC
CTGGAGCCCCTGGTGGACCTGCCCATCGGCATCAACATCACCAGATTCCAG
ACCCTGCTGGCCCTGCACAGAAGCTACCTGACCCCCGGCGACAGCAGCAGC
GGCTGGACCGCCGGCGCCGCCGCCTACTACGTGGGCTACCTGCAGCCCAG
AACCTTCCTGCTGAAGTACAACGAGAACGGCACCATCACCGACGCCGTGGA
CTGCGCCCTGGACCCCCTGAGCGAGACCAAGTGCACCCTGAAGAGCTTCAC
CGTGGAGAAGGGCATCTACCAGACCAGCAACTTCAGAGTGCAGCCCACCGA
GAGCATCGTGAGATTCCCCAACATCACCAACCTGTGCCCCTTCGGCGAGGT
GTTCAACGCCACCAGATTCGCCAGCGTGTACGCCTGGAACAGAAAGAGAAT
CAGCAACTGCGTGGCCGACTACAGCGTGCTGTACAACAGCGCCAGCTTCAG
CACCTTCAAGTGCTACGGCGTGAGCCCCACCAAGCTGAACGACCTGTGCTT
CACCAACGTGTACGCCGACAGCTTCGTGATCAGAGGCGACGAGGTGAGACA
GATCGCCCCCGGCCAGACCGGCAAGATCGCCGACTACAACTACAAGCTGCC
CGACGACTTCACCGGCTGCGTGATCGCCTGGAACAGCAACAACCTGGACAG
CAAGGTGGGCGGCAACTACAACTACCTGTACAGACTGTTCAGAAAGAGCAAC
CTGAAGCCCTTCGAGAGAGACATCAGCACCGAGATCTACCAGGCCGGCAGC
ACCCCCTGCAACGGCGTGGAGGGCTTCAACTGCTACTTCCCCCTGCAGAGC
TACGGCTTCCAGCCCACCAACGGCGTGGGCTACCAGCCCTACAGAGTGGTG
GTGCTGAGCTTCGAGCTGCTGCACGCCCCCGCCACCGTGTGCGGCCCCAA
GAAGAGCACCAACCTGGTGAAGAACAAGTGCGTGAACTTCAACTTCAACGGC
CTGACCGGCACCGGCGTGCTGACCGAGAGCAACAAGAAGTTCCTGCCCTTC
CAGCAGTTCGGCAGAGACATCGCCGACACCACCGACGCCGTGAGAGACCC
CCAGACCCTGGAGATCCTGGACATCACCCCCTGCAGCTTCGGCGGCGTGAG
CGTGATCACCCCCGGCACCAACACCAGCAACCAGGTGGCCGTGCTGTACCA
GGGCGTGAACTGCACCGAGGTGCCCGTGGCCATCCACGCCGACCAGCTGA
CCCCCACCTGGAGAGTGTACAGCACCGGCAGCAACGTGTTCCAGACCAGAG
CCGGCTGCCTGATCGGCGCCGAGCACGTGAACAACAGCTACGAGTGCGACA
TCCCCATCGGCGCCGGCATCTGCGCCAGCTACCAGACCCAGACCAACAGCC
CCAGAAGAGCCAGAAGCGTGGCCAGCCAGAGCATCATCGCCTACACCATGA
GCCTGGGCGCCGAGAACAGCGTGGCCTACAGCAACAACAGCATCGCCATCC
CCACCAACTTCACCATCAGCGTGACCACCGAGATCCTGCCCGTGAGCATGA
CCAAGACCAGCGTGGACTGCACCATGTACATCTGCGGCGACAGCACCGAGT
GCAGCAACCTGCTGCTGCAGTACGGCAGCTTCTGCACCCAGCTGAACAGAG
CCCTGACCGGCATCGCCGTGGAGCAGGACAAGAACACCCAGGAGGTGTTC
GCCCAGGTGAAGCAGATCTACAAGACCCCCCCCATCAAGGACTTCGGCGGC
TTCAACTTCAGCCAGATCCTGCCCGACCCCAGCAAGCCCAGCAAGAGAAGC
TTCATCGAGGACCTGCTGTTCAACAAGGTGACCCTGGCCGACGCCGGCTTC
ATCAAGCAGTACGGCGACTGCCTGGGCGACATCGCCGCCAGAGACCTGATC
TGCGCCCAGAAGTTCAACGGCCTGACCGTGCTGCCCCCCCTGCTGACCGAC
GAGATGATCGCCCAGTACACCAGCGCCCTGCTGGCCGGCACCATCACCAGC
GGCTGGACCTTCGGCGCCGGCGCCGCCCTGCAGATCCCCTTCGCCATGCA
GATGGCCTACAGATTCAACGGCATCGGCGTGACCCAGAACGTGCTGTACGA
GAACCAGAAGCTGATCGCCAACCAGTTCAACAGCGCCATCGGCAAGATCCA
GGACAGCCTGAGCAGCACCGCCAGCGCCCTGGGCAAGCTGCAGGACGTGG
TGAACCAGAACGCCCAGGCCCTGAACACCCTGGTGAAGCAGCTGAGCAGCA
ACTTCGGCGCCATCAGCAGCGTGCTGAACGACATCCTGAGCAGACTGGACA
AGGTGGAGGCCGAGGTGCAGATCGACAGACTGATCACCGGCAGACTGCAG
AGCCTGCAGACCTACGTGACCCAGCAGCTGATCAGAGCCGCCGAGATCAGA
GCCAGCGCCAACCTGGCCGCCACCAAGATGAGCGAGTGCGTGCTGGGCCA
GAGCAAGAGAGTGGACTTCTGCGGCAAGGGCTACCACCTGATGAGCTTCCC
CCAGAGCGCCCCCCACGGCGTGGTGTTCCTGCACGTGACCTACGTGCCCG
CCCAGGAGAAGAACTTCACCACCGCCCCCGCCATCTGCCACGACGGCAAGG
CCCACTTCCCCAGAGAGGGCGTGTTCGTGAGCAACGGCACCCACTGGTTCG
TGACCCAGAGAAACTTCTACGAGCCCCAGATCATCACCACCGACAACACCTT
CGTGAGCGGCAACTGCGACGTGGTGATCGGCATCGTGAACAACACCGTGTA
CGACCCCCTGCAGCCCGAGCTGGACAGCTTCAAGGAGGAGCTGGACAAGTA
CTTCAAGAACCACACCAGCCCCGACGTGGACCTGGGCGACATCAGCGGCAT
CAACGCCAGCGTGGTGAACATCCAGAAGGAGATCGACAGACTGAACGAGGT
GGCCAAGAACCTGAACGAGAGCCTGATCGACCTGCAGGAGCTGGGCAAGTA
CGAGCAGTACATCAAGTGGCCCTGGTACATCTGGCTGGGCTTCATCGCCGG
CCTGATCGCCATCGTGATGGTGACCATCATGCTGTGCTGCATGACCAGCTGC
TGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAA

### PNI A5

PNI A5 is based on full-length SARS COV 2 surface glycoprotein S from the NCBI database reference MN908947.3 and codon optimized for mammalian cell expression with the D614G dominant mutation incorporated, as described for PNI A4. However, additional single nucleotide base changes in the full codon optimized sequence were also made PNI A5 (Table 10).

TABLE 10

| Nucleotide | Original base in | Modified base in |
|---|---|---|
| position | PNI A4 | PNI A5 |
| 291 | G | A |
| 912 | G | A |
| 1584 | G | A |
| 2046 | A | G |
| 3821 | G | A |

The full codon optimized sequence with the changes is provided below.

SEQ ID NO: 7
PNI A5

ATGTTCGTGTTCCTGGTGCTGCTGCCCCTGGTGAGCAGCCAGTGCGTGAAC
CTGACCACCAGAACCCAGCTGCCCCCCGCCTACACCAACAGCTTCACCAGA
GGCGTGTACTACCCCGACAAGGTGTTCAGAAGCAGCGTGCTGCACAGCACC
CAGGACCTGTTCCTGCCCTTCTTCAGCAACGTGACCTGGTTCCACGCCATCC
ACGTGAGCGGCACCAACGGCACCAAGAGATTCGACAACCCCGTGCTGCCCT
TCAACGACGGCGTGTACTTCGCCAGCACCGAGAAAAGCAACATCATCAGAG
GCTGGATCTTCGGCACCACCCTGGACAGCAAGACCCAGAGCCTGCTGATCG
TGAACAACGCCACCAACGTGGTGATCAAGGTGTGCGAGTTCCAGTTCTGCAA
CGACCCCTTCCTGGGCGTGTACTACCACAAGAACAACAAGAGCTGGATGGA
GAGCGAGTTCAGAGTGTACAGCAGCGCCAACAACTGCACCTTCGAGTACGT
GAGCCAGCCCTTCCTGATGGACCTGGAGGGCAAGCAGGGCAACTTCAAGAA
CCTGAGAGAGTTCGTGTTCAAGAACATCGACGGCTACTTCAAGATCTACAGC
AAGCACACCCCCATCAACCTGGTGAGAGACCTGCCCCAGGGCTTCAGCGCC
CTGGAGCCCCTGGTGGACCTGCCCATCGGCATCAACATCACCAGATTCCAG
ACCCTGCTGGCCCTGCACAGAAGCTACCTGACCCCCGGCGACAGCAGCAGC
GGCTGGACCGCCGGCGCCGCCGCCTACTACGTGGGCTACCTGCAGCCCAG
AACCTTCCTGCTGAAGTACAACGAGAACGGCACCATCACCGACGCCGTGGA
CTGCGCCCTGGACCCCCTGAGCGAGACCAAGTGCACCCTGAAAAGCTTCAC
CGTGGAGAAGGGCATCTACCAGACCAGCAACTTCAGAGTGCAGCCCACCGA
GAGCATCGTGAGATTCCCCAACATCACCAACCTGTGCCCCTTCGGCGAGGT
GTTCAACGCCACCAGATTCGCCAGCGTGTACGCCTGGAACAGAAAGAGAAT
CAGCAACTGCGTGGCCGACTACAGCGTGCTGTACAACAGCGCCAGCTTCAG
CACCTTCAAGTGCTACGGCGTGAGCCCCACCAAGCTGAACGACCTGTGCTT
CACCAACGTGTACGCCGACAGCTTCGTGATCAGAGGCGACGAGGTGAGACA
GATCGCCCCCGGCCAGACCGGCAAGATCGCCGACTACAACTACAAGCTGCC
CGACGACTTCACCGGCTGCGTGATCGCCTGGAACAGCAACAACCTGGACAG
CAAGGTGGGCGGCAACTACAACTACCTGTACAGACTGTTCAGAAAGAGCAAC
CTGAAGCCCTTCGAGAGAGACATCAGCACCGAGATCTACCAGGCCGGCAGC
ACCCCCTGCAACGGCGTGGAGGGCTTCAACTGCTACTTCCCCCTGCAGAGC
TACGGCTTCCAGCCCACCAACGGCGTGGGCTACCAGCCCTACAGAGTGGTG
GTGCTGAGCTTCGAGCTGCTGCACGCCCCCGCCACCGTGTGCGGCCCCAAA
AAGAGCACCAACCTGGTGAAGAACAAGTGCGTGAACTTCAACTTCAACGGCC
TGACCGGCACCGGCGTGCTGACCGAGAGCAACAAGAAGTTCCTGCCCTTCC
AGCAGTTCGGCAGAGACATCGCCGACACCACCGACGCCGTGAGAGACCCC
CAGACCCTGGAGATCCTGGACATCACCCCCTGCAGCTTCGGCGGCGTGAGC
GTGATCACCCCCGGCACCAACACCAGCAACCAGGTGGCCGTGCTGTACCAG
GGCGTGAACTGCACCGAGGTGCCCGTGGCCATCCACGCCGACCAGCTGAC
CCCCACCTGGAGAGTGTACAGCACCGGCAGCAACGTGTTCCAGACCAGAGC
CGGCTGCCTGATCGGCGCCGAGCACGTGAACAACAGCTACGAGTGCGACAT
CCCCATCGGCGCCGGCATCTGCGCCAGCTACCAGACCCAGACCAACAGCCC
CAGGAGAGCCAGAAGCGTGGCCAGCCAGAGCATCATCGCCTACACCATGAG
CCTGGGCGCCGAGAACAGCGTGGCCTACAGCAACAACAGCATCGCCATCCC
CACCAACTTCACCATCAGCGTGACCACCGAGATCCTGCCCGTGAGCATGAC
CAAGACCAGCGTGGACTGCACCATGTACATCTGCGGCGACAGCACCGAGTG
CAGCAACCTGCTGCTGCAGTACGGCAGCTTCTGCACCCAGCTGAACAGAGC
CCTGACCGGCATCGCCGTGGAGCAGGACAAGAACACCCAGGAGGTGTTCG
CCCAGGTGAAGCAGATCTACAAGACCCCCCCCATCAAGGACTTCGGCGGCT
TCAACTTCAGCCAGATCCTGCCCGACCCCAGCAAGCCCAGCAAGAGAAGCT
TCATCGAGGACCTGCTGTTCAACAAGGTGACCCTGGCCGACGCCGGCTTCA
TCAAGCAGTACGGCGACTGCCTGGGCGACATCGCCGCCAGAGACCTGATCT
GCGCCCAGAAGTTCAACGGCCTGACCGTGCTGCCCCCCCTGCTGACCGACG
AGATGATCGCCCAGTACACCAGCGCCCTGCTGGCCGGCACCATCACCAGCG
GCTGGACCTTCGGCGCCGGCGCCGCCCTGCAGATCCCCTTCGCCATGCAG
ATGGCCTACAGATTCAACGGCATCGGCGTGACCCAGAACGTGCTGTACGAG
AACCAGAAGCTGATCGCCAACCAGTTCAACAGCGCCATCGGCAAGATCCAG
GACAGCCTGAGCAGCACCGCCAGCGCCCTGGGCAAGCTGCAGGACGTGGT
GAACCAGAACGCCCAGGCCCTGAACACCCTGGTGAAGCAGCTGAGCAGCAA
CTTCGGCGCCATCAGCAGCGTGCTGAACGACATCCTGAGCAGACTGGACAA
GGTGGAGGCCGAGGTGCAGATCGACAGACTGATCACCGGCAGACTGCAGA
GCCTGCAGACCTACGTGACCCAGCAGCTGATCAGAGCCGCCGAGATCAGAG
CCAGCGCCAACCTGGCCGCCACCAAGATGAGCGAGTGCGTGCTGGGCCAG
AGCAAGAGAGTGGACTTCTGCGGCAAGGGCTACCACCTGATGAGCTTCCCC
CAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGC
CCACTTCCCCAGAGAGGGCGTGTTCGTGAGCAACGGCACCCACTGGTTCGT
GACCCAGAGAAACTTCTACGAGCCCCAGATCATCACCACCGACAACACCTTC
GTGAGCGGCAACTGCGACGTGGTGATCGGCATCGTGAACAACACCGTGTAC
GACCCCCTGCAGCCCGAGCTGGACAGCTTCAAGGAGGAGCTGGACAAGTAC
TTCAAGAACCACACCAGCCCCGACGTGGACCTGGGCGACATCAGCGGCATC
AACGCCAGCGTGGTGAACATCCAGAAGGAGATCGACAGACTGAACGAGGTG
GCCAAGAACCTGAACGAGAGCCTGATCGACCTGCAGGAGCTGGGCAAGTAC
GAGCAGTACATCAAGTGGCCCTGGTACATCTGGCTGGGCTTCATCGCCGGC
CTGATCGCCATCGTGATGGTGACCATCATGCTGTGCTGCATGACCAGCTGCT
GCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAA

### PNI A6

PNI A6 is based on SARS COV2 surface glycoprotein S from the NCBI database reference NC_045512.2. The following mutations (shown in Table 11) were made IN the original sequence to get the desired E484K, N501Y, D614G, K986P and K987P.

TABLE 11

| Desired amino | Nucleotide | Original | Modified |
|---|---|---|---|
| acid change | position | base | base |
| E484K | 1450 | G | A |
| N501Y | 1501 | A | T |
| D614G | 1841 | A | G |
| D614G | 1842 | T | C |
| K986P | 2956 | A | C |
| K986P | 2957 | A | C |
| K987P | 2958 | G | C |
| K986P | 2959 | T | C |

### PNI A8

PNI A8 is based on SARS COV2 surface glycoprotein S from the NCBI database reference NC_045512.2. The following mutations (Table 12) were made in the original sequence to achieve the desired del19H, del20V, delL144Y, S155P, E484K, N501Y, D614G, K986P and K987P.

TABLE 12

| Desired amino | Nucleotide | Original | Modified |
|---|---|---|---|
| acid change | position | base | base |
| Del69H | 205, 206, 207 | C, A, T | — |
| Del70V | 208, 209, 210 | G, T, C | — |
| Delta144Y | 430, 431, 432 | T, A, T | — |
| S155P | 463 | A | C |
| S155P | 464 | G | C |
| E484K | 1450 | G | A |
| N501Y | 1501 | A | T |
| D614G | 1841 | A | G |
| D614G | 1842 | T | C |
| K986P | 2956 | A | C |
| K986P | 2957 | A | C |
| K987P | 2958 | G | C |
| K986P | 2959 | T | C |

### PNI A9

PNI A9 is based on SARS COV2 surface glycoprotein S from the NCBI database reference NC_045512.2. In detail, it is a modified version of PNI A6 with a tPA signal sequence (ATGgacgccatgaagcggggcctctgctgtgttctgctgctctgcggcgccgtgttcgtgagtaactcg) (SEQ ID NO: 8) at the N terminal.

### PNI A10

PNI A10 is based on the reference SARS COV2 surface glycoprotein S from the NCBI database reference NC_045512.2. It is a modified sequence encoding NC_045512.2 with a P2A sfGFP sequence (Table 13) at the C-terminal end.

TABLE 13
SEQ ID NO: 9
P2A sfGFP sequence

ATGGGAAGCG GAATGGGAAG CGGAGCCACG AACTTCTCTC TGTTAAAGCA
AGCAGGAGAT GTTGAAGAAA ACCCCGGGCC TATGAGCAAA
GGAGAAGAAC
TTTTCACTGG AGTTGTCCCA ATTCTTGTTG AATTAGATGG TGATGTTAAT
GGGCACAAAT TTTCTGTCCG TGGAGAGGGT GAAGGTGATG CTACAAACGG
AAAACTCACC CTTAAATTTA TTTGCACTAC TGGAAAACTA CCTGTTCCGT
GGCCAACACT TGTCACTACT CTGACCTATG GTGTTCAATG CTTTTCCCGT
TATCCGGATC ACATGAAACG GCATGACTTT TTCAAGAGTG CCATGCCCGA
AGGTTATGTA CAGGAACGCA CTATATCTTT CAAAGATGAC GGGACCTACA
AGACGCGTGC TGAAGTCAAG TTTGAAGGTG ATACCCTTGT TAATCGTATC
GAGTTAAAGG GTATTGATTT TAAAGAAGAT GGAAACATTC TTGGACACAA
ACTCGAGTAC AACTTTAACT CACACAATGT ATACATCACG GCAGACAAAC
AAAAGAATGG AATCAAAGCT AACTTCAAAA TTCGCCACAA CGTTGAAGAT
GGTTCCGTTC AACTAGCAGA CCATTATCAA CAAAATACTC CAATTGGCGA
TGGCCCTGTC CTTTTACCAG ACAACCATTA CCTGTCGACA CAATCTGTCC
TTTCGAAAGA TCCCAACGAA AAGCGTGACC ACATGGTCCT TCTTGAGTTT
GTAACTGCTG CTGGGATTAC ACATGGCATG GATGAGCTCT ACAAATAA

### PNI A11

PNI A11 is based on the reference SARS COV2 surface glycoprotein S from the NCBI database reference NC_045512.2. It has the N-terminal Domain (NTD), the Receptor binding domain (RBD), the trans-membrane™ and the C-terminal domain (CTD) of the original NC_045512.2 sequence (Table 14).

TABLE 14

| Domain | Sequence position | Sequence length |
|---|---|---|
| NTD | 1-905 | 906 |
| RBD | 906-1623 | 717 |
| TM | 3637-3711 | 74 |
| CTD | 3711-3822 | 111 |

### PNI A12

PNI A12 is based on SARS COV2 surface glycoprotein S from the NCBI database reference NC_045512.2. It is a modified version of the PNI All sequence with L18F, T20N, P26S, del 69-70HV, del144Y, D138Y, R190S, K417N, E484K and N501Y modifications (RBD Mut).

TABLE 15

| Desired amino | Nucleotide | Original | Modified |
|---|---|---|---|
| acid change | position | base | base |
| L18F | 52 | C | T |
| T20N | 60 | C | A |
| P26S | 76 | C | T |
| Del69H | 205, 206, 207 | C, A, T | — |
| Del70V | 208, 209, 210 | G, T, C | — |
| D138Y | 412 | G | T |
| Del144Y | 430, 431, 432 | T, A, T | — |
| R190S | 570 | G | C |
| K417N | 1251 | G | A |
| E484K | 1450 | G | A |
| N501Y | 1501 | A | T |

### PNI A13

PNI A13 (Table 17) is based on SARS COV2 surface glycoprotein S from the NCBI database reference NC_045512.2. It is a modified version of the PNI A12 sequence with the tPA signal sequence (69 bp: as described with respect to the PNI A9 sequence) at the N-terminus and a linker-fibritin foldon sequence (Table 16) between the RBD and the TM-CTD domain.

TABLE 16
SEQ ID NO: 10
Linker-Fibritin foldon sequence

TCTGGTGGCG GAGGCTCGGG CGGAGGTGGG TCGGGTGGCG
GCGGATCAGG
TTATATTCCT GAAGCTCCAA GAGATGGGCA AGCTTACGTT CGTAAAGATG
GCGAATGGGT ATTCCTTTCT ACCTTTTTAT CACCAGCA

TABLE 17
PNI A13: tPA-NTD-RBD-linker-fibritin foldon-TM-CTD.

| Domain | Sequence position | Sequence length |
|---|---|---|
| tPA | 1-69 | 69 |
| NTD-RBD Mut (PNI A12) | 70-1680 | 1610 |
| Linker | 1681-1728 | 47 |
| Fibritin foldon | 1729-1818 | 89 |
| TM-CTD | 1819-2004 | 185 |

### PNI A14

PNI A14 (Table 19) SARS COV2 surface glycoprotein S from the NCBI database reference NC_045512.2. It is a modified version of the PNI All sequence with the tPA signal sequence (69 bp: as described above with respect to PNI A9) at the N-terminus and SS (Table 18), WT RBD and TM-CTD as in NC_045512.2.

TABLE 18
SEQ ID NO: 11
PNI A14
Signal sequence (SS)

ATGTTTGTTT TTCTTGTTTT ATTGCCACTA GTCTCTAGTC AGTGTGTTAA
TCTTACAACC AGAACTCAAT TACCCCCTGC ATACACTAAT TOTTTCACAC G

TABLE 19
PNI A14

| Domain | Sequence position | Sequence length |
|---|---|---|
| tPA | 1-69 | 69 |
| SS | 70-167 | 98 |
| WT RBD | 167-885 | 718 |
| TM-CTD | 886-1071 | 185 |

### PNI A15

PNI A15 (Table 20) is based on SARS COV2 surface glycoprotein S from the NCBI database reference NC_045512.2. It is a modified version of the PNI A12 sequence with the tPA signal sequence (69 bp: as described with respect to PNI A9) at the N-terminus and signal sequence (SS), RBD Mut (PNI A12 RBD) and TM-CTD from NC_045512.2.

TABLE 20

| Domain | Sequence position | Sequence length |
|---|---|---|
| tPA | 1-69 | 69 |
| SS | 70-167 | 98 |
| RBD Mut (PNI A12 RBD) | 167-885 | 718 |
| TM-CTD | 886-1071 | 185 |

### PNI A16

PNI A16 is based on SARS COV2 surface glycoprotein S from the NCBI database reference NC_045512.2. It is a modified version of PNI A15 with two additional 3′ untranslated regions (UTR) from amino enhancer of split (AES) (Table 21) and mitochondrially encoded 12S RRNA (mt-RNR1) (Table 22).

TABLE 21
SEQ ID NO: 12
3′ UTR AES

CTGGTACTGC ATGCACGCAA TGCTAGCTGC CCCTTTCCCG TCCTGGGTAC
CCCGAGTCTC CCCCGACCTC GGGTCCCAGG TATGCTCCCA
CCTCCACCTG
CCCCACTCAC CACCTCTGCT AGTTCCAGAC ACCTCC

TABLE 22
SEQ ID NO: 13
3′ UTR mtRNR1

CAAGCACGCA GCAATGCAGC TCAAAACGCT TAGCCTAGCC ACACCCCCAC
GGGAAACAGC AGTGATTAAC CTTTAGCAAT AAACGAAAGT TTAACTAAGC
TATACTAACC CCAGGGTTGG TCAATTTCGT GCCAGCCACA CC

### PNI A17

PNI A17 (Table 23) is based on SARS COV2 surface glycoprotein S from the NCBI database reference NC_045512.2. It incorporates the mutations of the following variants:
P1: K417N/T, E484K, N501Y, D614G
B.1.351: K417N, E484K, N501Y, D614G
B.1.427: L452R, D614G
B.1.429: S13I, W152C, L452R, D614G
K986P, V987P

TABLE 23
PNI A17

| Desired amino | Nucleotide | Original | Modified |
|---|---|---|---|
| acid change | position | base | base |
| S13I | 37 | G | T |
| W152C | 456 | G | C |
| K417N | 1251 | G | A |
| L452R | 1355 | T | G |
| E484K | 1450 | G | A |
| N501Y | 1501 | A | T |
| D614G | 1841 | A | G |
| D614G | 1842 | T | C |
| K986P | 2956 | A | C |
| K986P | 2957 | A | C |
| K987P | 2958 | G | C |
| K986P | 2959 | T | C |

### PNI A18

PNI A18 is based on SARS COV2 surface glycoprotein S from the NCBI database reference NC_045512.2. It incorporates the mutations of the following variants: B 1.351 strain mutations: D80A, D215G, del 241, del 242, del 243, K417N, E484K, N501Y, D614G, A701V, K986P, and V987P.

TABLE 24
PNI A18

| Desired amino | Nucleotide | Original | Modified |
|---|---|---|---|
| acid change | position | base | base |
| D80A | 239 | A | C |
| D215G | 644 | A | G |
| Del241 | 721, 722, 723 | T, T, A | — |
| Del242 | 724, 725, 726 | C, T, T | — |
| Del243 | 727, 728, 729 | G, C, T | — |
| K417N | 1251 | G | A |
| E484K | 1450 | G | A |
| N501Y | 1501 | A | T |
| D614G | 1841 | A | G |
| D614G | 1842 | T | C |
| A701V | 2102 | C | T |
| K986P | 2956 | A | C |
| K986P | 2957 | A | C |
| K987P | 2958 | G | C |
| K986P | 2959 | T | C |

### PNI A19

PNI A19 (Table 25) is based on SARS COV2 surface glycoprotein S from the NCBI database reference NC_045512.2. It incorporates the mutations of the following variants: B 1.617 strain-452R, 484Q, K986P, V987P, 144del, 478K, 69del, and 70del.

TABLE 25
PNI A19

| Desired amino | Nucleotide | Original | Modified |
|---|---|---|---|
| acid change | position | base | base |
| Del69H | 205, 206, 207 | C, A, T | — |
| Del70V | 208, 209, 210 | G, T, C | — |
| Del144Y | 430, 431, 432 | T, A, T | — |
| L452R | 1355 | T | G |
| T478K | 1433 | C | A |
| E484Q | 1450 | G | C |
| K986P | 2956 | A | C |
| K986P | 2957 | A | C |
| K987P | 2958 | G | C |
| K986P | 2959 | T | C |

### PNI A20

PNI A20 is based on SARS COV2 surface glycoprotein S from the NCBI database reference NC_045512.2. It is the modified PNI A19 with tPA signal sequence at the N terminus and 3′ AES UTR and 3′ mtRNR1 UTRs at the end after the stop codon.

### PNI A21

PNI A21 (Table 26) is based on SARS COV2 surface glycoprotein S from the NCBI database reference NC_045512.2. It incorporates the mutations of the following variants: B.1.617.2 strain: T19R, (G142D*), 156del, 157del, R158G, L452R, T478K, D614G, P681R, and D950N.

TABLE 26
PNI A21

| Desired amino | Nucleotide | Original | Modified |
|---|---|---|---|
| acid change | position | base | base |
| T19R | 56 | C | G |
| Del156 | 466, 467, 468 | G, A, G | — |
| Del157 | 469, 470, 471 | T, T, C | — |
| R158G | 472 | A | G |
| L452R | 1355 | T | G |
| T478K | 1433 | C | A |
| D614G | 1841 | A | G |
| D614G | 1842 | T | C |
| P681R | 2041 | C | G |
| P681R | 2042 | T | G |
| D950N | 2847 | G | A |
| K986P | 2956 | A | C |
| K986P | 2957 | A | C |
| K987P | 2958 | G | C |
| K986P | 2959 | T | C |

### Example 2

This example demonstrates the microfluidic mixing of nucleic acid therapeutics (NAT) into Lipid Nanoparticles (LNP).

The vectors described in Example 1 were independently diluted using sodium acetate buffer to the required concentration. Lipid nucleic acid particle (LNAP) samples were then prepared as described by running both fluids using the NANOASSEMBLR instrument. Briefly, 10-20 μg of nucleic acids in 100 mM sodium acetate buffer in a total volume of 32 μL was mixed with 16 μL of 37.5 mM lipid mix solution (see Table 27) as required by the N/P ratios (4, 6, or 10 in illustrated examples) in a NANOASSEMBLR Spark™ instrument. The resulting lipid nucleic acid particles (LNAP) were immediately diluted down with 48 μL Ca++ and Mg++free 1X PBS at pH 7.4 in the aqueous output well. These LNAP were immediately collected into microcentrifuge tubes containing 96 μL of the same buffer at pH 7.4. Encapsulation efficiency was measured by a modified Ribogreen™ assay (Quanti-iT RiboGreen™ RNA assay kit, Fisher). This information was used to establish the desired dosage.

Lipid particles were also manufactured by a larger microfluidic mixer instrument, the NANOASSEMBLR Ignite™ instrument for testing. Briefly, 350 μL of mRNA was diluted using 100 mM sodium acetate buffer to the required concentration of 0.2 to 0.3 mg/m. A lipid mix solution of 12.5 or 25 mM was typically used. LNAP were then prepared by running both fluids, namely, nucleic acids in aqueous solvent and Lipid Mix in ethanol at a flow ratio of 3:1 and at a total flow rate of 12 ml/min in the microfluidic mixer. Following mixing in the microfluidic device, the post cartridge lipid nucleic acid particle sample was diluted into RNAse free tubes containing three to 40 volumes of PBS, pH 7.4. Ethanol was finally removed through either dialysis in PBS, pH 7, or using Amicon™ centrifugal filters (Millipore, USA) at 3000 RPM, or using TFF systems. Once the required concentration was achieved, the lipid nucleic acid particles were filter sterilized using 0.2 μm filters in aseptic conditions. Final encapsulation efficiency was measured by the Ribogreen™ assay. Quant-iT™ RIBOGREEN RNA Reagent and Kit (Invitrogen) following manufacturer directions. Self amplifying mRNA LNP preparation is described below. Observed particle attributes were generally sized from 50-200 nm for mRNA, depending on lipid composition.

In Table 27 are listed possible lipid mixes for use with vaccines according to aspects of the invention. VACCMIX-A or VACCMIX-B were used in the described experiments. iL=ionizable lipid. Other acronyms are defined below Table 27.

TABLE 27
Exemplary Lipid Mixes
Lipid Combination and Ratios

VACCMIX-A: 47.5 mol % IL/12.5 mol % DSPC/38.5 mol % Chol/1.5 mol % PEG-DMG
VACCMIX-B: 47.5 mol % IL/12.5 mol % DOPE/38.5 mol % Chol/1.5 mol % PEG-DMG
50 mol % IL/10 mol % DSPC/37.4 mol % Chol/2.5 mol % Polyoxyethylene (40)
stearate
50 mol % IL/10 mol % DSPC/38.5 mol % Chol/1.5 mol % PEG-DMG
47.5 mol % IL/12.5 mol % DOPC/38.5 mol % Chol/1.5 mol % PEG-DMG
50 mol % IL/10 mol % DOPC/38.5 mol % Chol/1.5 mol % PEG-DMG 2000
40 mol % IL/40 mol % DOPE/17.5 mol % Chol/2.5 mol % Polyoxyethylene (40)
stearate
40 mol % IL/30 mol % DOPE/17 mol % Chol/10 mol % Triolein/2.5 mol % Stabilizer
40 mol % IL/20 mol % DOPE/17.5 mol % Chol/20 mol % Triolein/2.5 mol % Stabilizer
40 mol % IL/40 mol % DOPE/0 mol % Chol/17.4 mol % Triolein/2.5 mol % Stabilizer
40 mol % IL/20 mol % DOPE/37.5 mol % Chol/2.5 mol % Tridecyl -D-maltoside
40 mol % IL/20 mol % DOPE/37.5 mol % Chol/2.5 mol % Tween 20
40 mol % IL/20 mol % DOPE/37.5 mol % Chol/2.5 mol % Polysorbate 80
40 mol % IL/20 mol % DOPE/37.5 mol % Chol/2.5 mol % Lipid H
40 mol % IL/20 mol % DOPE/35.9 mol % Chol/4 mol % Lipid H
50 mol % IL/10 mol % DSPC/37.5 mol % Chol/2.5 mol % BRIJ ™ S20
50 mol % IL/10 mol % DSPC/40 mol % Chol/2.5 mol % TPGS1000
50 mol % IL/10 mol % DSPC/37.5 mol % Chol/2.5 mol % BRIJ ™S10
50 mol % IL/10 mol % DSPC/38.5 mol % Chol/1.5 mol % BRIJ ™S10
50 mol % IL/10 mol % DSPC/37.5 mol % Chol/2.5 mol % Polyoxyethylene (4) lauryl
ether
50 mol % IL/10 mol % DSPC/38.5 mol % Chol/1.5 mol % Polysorbate 80
50 mol % IL/10 mol % DSPC/37.5 mol % Chol/2.5 mol % Polysorbate 80
50 mol % IL/10 mol % DSPC/39.5 mol % Chol/0.5 mol % Polysorbate 80
40 mol % IL/20 mol % DSPC/37.5 mol % Chol/2.5 mol % BRIJ ™ S10
40 mol % IL/20 mol % DSPC/39.5 mol % Chol/0.5 mol % TPGS1000
40 mol % IL/20 mol % DSPC/39.5 mol % Chol/0.5 mol % BRIJ ™S10
40 mol % IL/20 mol % DSPC/38.5 mol % Chol/1.5 mol % Polysorbate 80
40 mol % IL/20 mol % DSPC/39.5 mol % Chol/0.5 mol % TPGS 1000
50 mol % C12-200/10 mol % DSPC/38.5 mol % Chol/1.5 mol % PEG-DMG
45 mol % IL/15 mol % DSPC/38.5 mol % Chol/1.5 mol % PEG-DMG
45 mol % IL/12.5 mol % DSPC/41 mol % Chol/1.5 mol % PEG-DMG
50 mol % IL/10 mol % DOPC/37.5 mol % Chol/2.5 mol % Polyoxyethylene (40)
stearate)
50 mol % IL/10 mol % DOPE/37.5 mol % Chol/2.5 mol % Polyoxyethylene (40)
stearate)
50 mol % IL/20 mol % DOPE/27.5 mol % Chol/2.5 mol % Polyoxyethylene (40)
stearate)
40 mol % IL/20 mol % DOPE/37.5 mol % Chol/2.5 mol % Polyoxyethylene (40)
stearate)
35 mol % IL/25 mol % DOPE/37.5 mol % Chol/2.5 mol % Polyoxyethylene (40)
stearate)/
40 mol % IL/20 mol % DOPE/39 mol % Chol/1 mol % Polyoxyethylene (40) stearate)/
40 mol % IL/40 mol % DOPE/17.5 mol % Chol/2.5 mol % Polyoxyethylene (40)
stearate)/
40 mol % IL/20 mol % DOPE & 20 mol % POPC/17 mol % Chol/2.5 mol %
Polyoxyethylene (40) stearate)/0.5 mol % DiD
40 mol % IL/40 mol % DOPE/17.5 mol % Chol/2.5 mol % Polyoxyethylene (40)
stearate
50 mol % IL/30 mol % DOPE/19 mol % Chol/1 mol % Polyoxyethylene (40) stearate)
50 mol % IL/10 mol % DSPE/38.5 mol % Chol/1.5 mol % PEG-DMG 2000
50 mol % IL/10 mol % DSPC/38.5 mol % Chol/1.5 mol % PEG-DMG 2000
47.5 mol % IL/12.5 mol % DSPC/38.5 mol % Chol/1.5 mol % PEG-DMG
47.5 mol % IL/12.5 mol % DPPC/38.5 mol % Chol/1.5 mol % PEG-DMG
Chol = cholesterol

### Example 3

This example demonstrates the characterization and encapsulation of Lipid Nucleic Acid Particle or “LNP.”

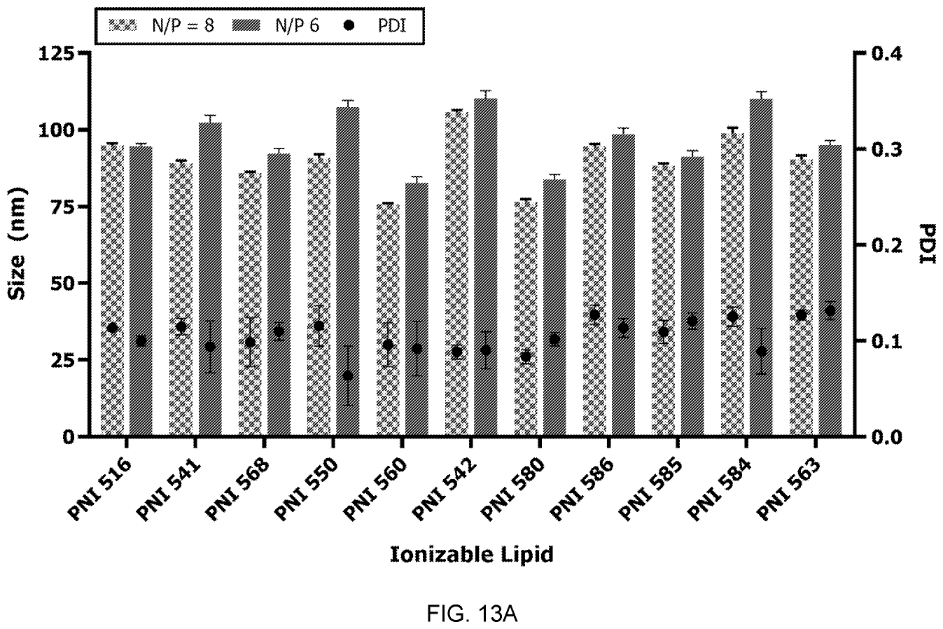

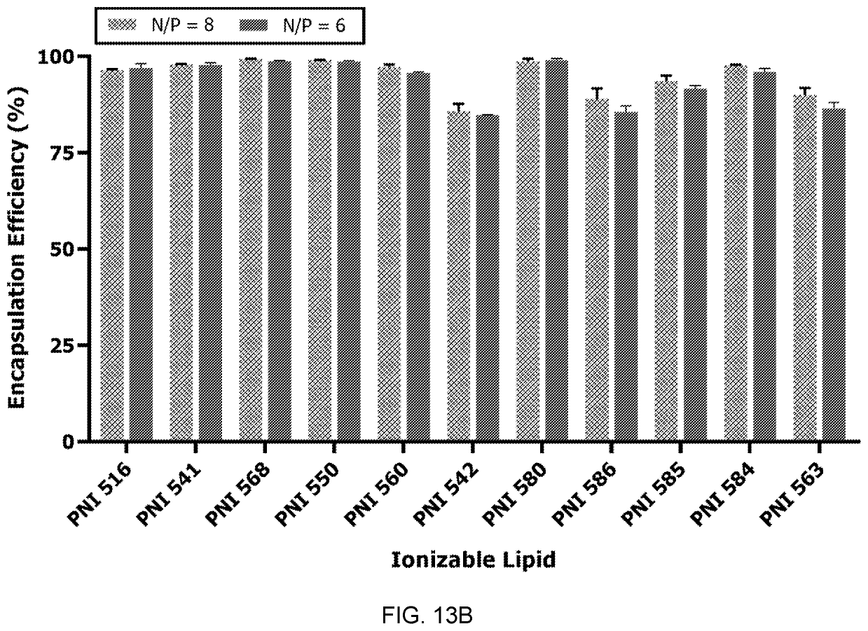

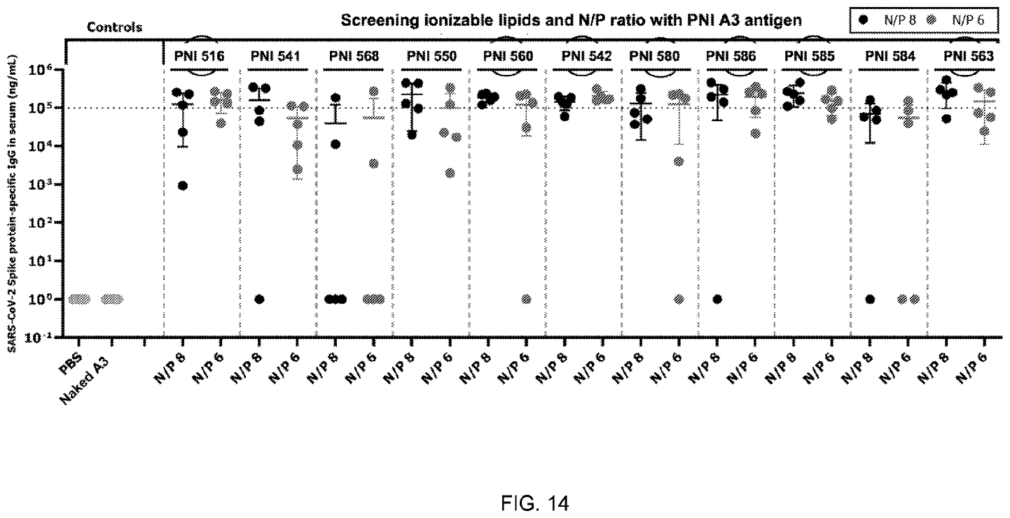

After the lipid particles were made as described above, the LNAP particle size (hydrodynamic diameter of the particles) was determined by Dynamic Light Scattering (DLS) using a ZetaSizer™ Nano ZS™ instrument (Malvern Instruments, UK). He/Ne laser of 633 nm wavelength was used as the light source. Data were measured from the scattered intensity data conducted in backscattering detection mode (measurement angle=173). Measurements were an average of 10 runs of two cycles each per sample. Z-Average size was reported as the particle size, and is defined as the harmonic intensity averaged particle diameter. Encapsulation efficiency was measured by a modified Ribogreen™ assay (Quanti-iT RiboGreen™ RNA assay kit, Fisher). There was good encapsulation in all of the formulations, with polydispersity (PDI) under 0.3. Results of sizing, PDI, and encapsulation efficiency for certain formulations wherein comparison studies were needed are shown in FIG. 13A, FIG. 13B, and FIG. 14. In other situations, size, PDI and EE were as expected or are illustrated in tabular form.

### Example 4

This example demonstrates the preparation of vector PNI V101.

The custom synthetic cloning vector PNI V101 for self-amplifying mRNA (saRNA) vaccines is the genetic machinery into which the Self amplifying mRNA is integrated. The product is herein referred to as “nCOV PNI V101.” It is a novel combination of self-replicating machinery along with a Poly (A) tail and multiple cloning sites incorporated within the cloning vector nCOV PNI V101. An illustration of the structure of the replicon is shown in FIG. 1. Self-replicating machinery along with Poly (A) tail with multiple cloning sites incorporated within the cloning vector nCOV PNI V101 enable the synthesis of large size saRNA that can generate naked positive strand alphavirus RNA replicons containing the gene of interest (GOI) both in vitro or in vivo. In FIG. 2, an alternative nCOV PNI V101 is shown in which the PNI V101 plasmid has an insertion of a 3822 bp gene sequence encoding for SARS-COV-2 spike protein which undergoes Pmel linearization. This vector generates an 11-nucleotide blunt end overhang after the poly (A) tail (top of FIG. 2), whereas BspQI restriction enzyme linearizes right at the end of the poly (A) tail producing a staggered end of 3 thymine nucleotides (bottom of FIG. 2)

The NanoOrange™ Protein Quantitation Kit (Invitrogen) was used in early testing, but the final product was assessed by a CE Fragment analyzer to be eGFP PNI V101 saRNA with a 5′ UTR and a 3′UTR. The product is an eGFP PNI V101 saRNA Plasmid DNA 8463 nt.

### Example 5

This example demonstrates the in vitro potency, size, PDI and encapsulation efficiency of an saRNA-based vaccine encoding SARS-COV-2 spike protein.

An saRNA-based vaccine encoding SARS-COV-2 spike protein was generated as the NAT for formulation within the LNP. The antigens were selected according to manufacturability. The nucleic acids encoding the antigens were codon optimized and tested. PNI A1, A2 and A4 did not prove capable of manufacture due to the ability to linearize plasmid. The sequences are shown in Example 1.

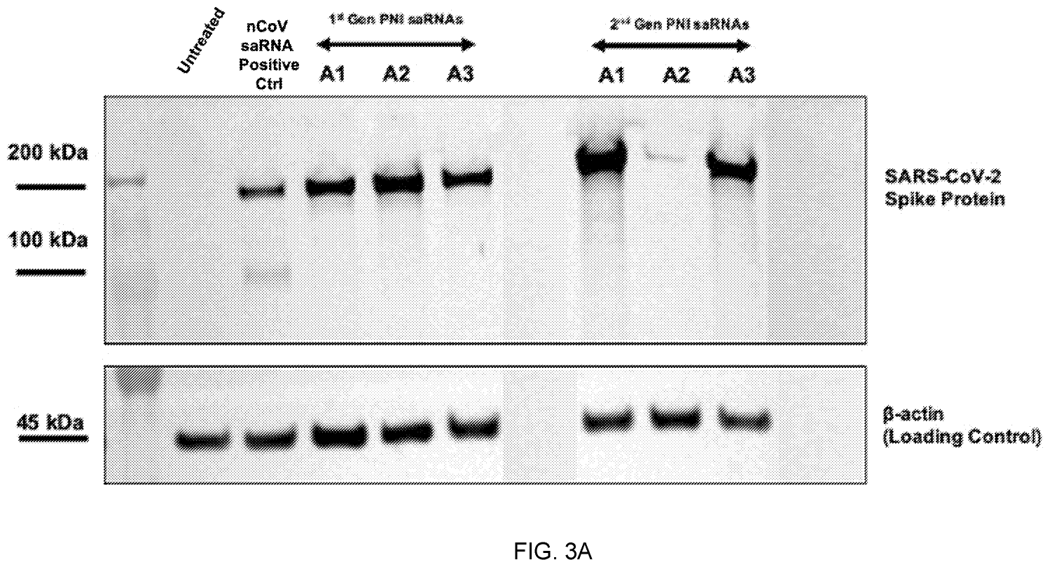

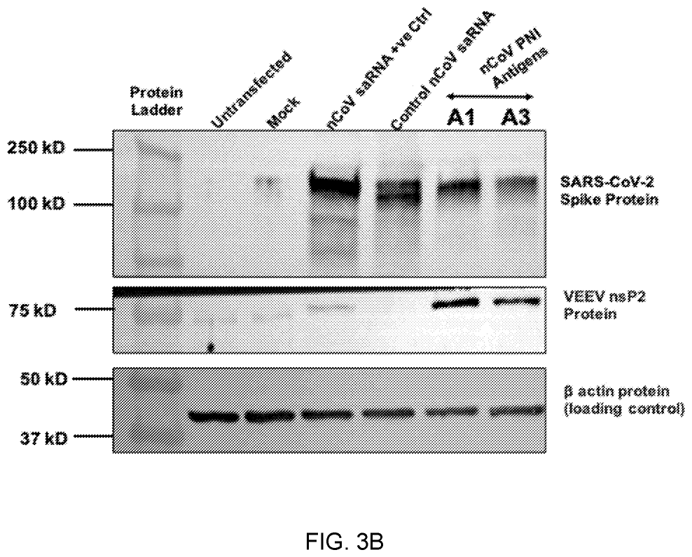

In vitro potency assays were performed using HEK293 and BHK-570 cells propagated from ATCC. An electrophoresis gel was performed using Beta actin, no transfection, and a positive control commercial spike protein sequence nCov CleanCap AU (Trilink) as controls. The PNI-v101 (NAT) encoding an nCOV antigen was microfluidically combined with PNI 516 VACCMIX-A as described supra. The gel in FIG. 3A shows that differences in SARS-COV-2 spike protein expression were observed between vectors with the Pmel and BspQ1 restriction sites. The protein bands shown in FIG. 3B indicate that the vector is intact, which correspond with protein expression for VEEV nsp2 as well as antigens A1 and A3.

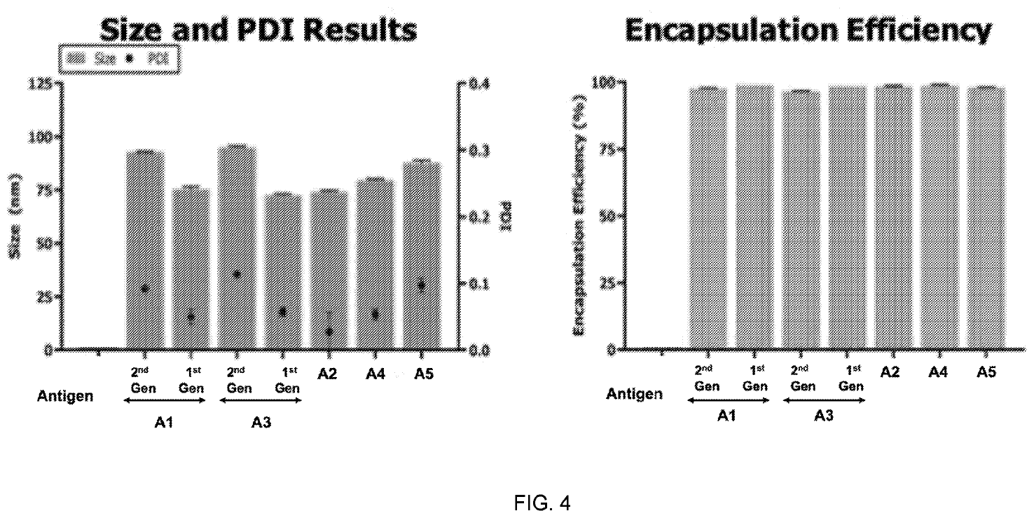

Size, PDI and encapsulation efficiency for nCov PNI V101 A3 are shown in FIG. 4. Other antigens performed similarly (results not shown).

### Example 6

This example demonstrates the results of administering the LNP vaccines of Example 2 to mice in vivo.

Vector NCOV PNI V101 LNP vaccines comprising saRNA-generating genes of interest, the 1^{st} and 2^{nd} generation of A1, A3 along with A2, A4 and A5, were screened in vivo. Mice were inoculated on day zero and then given a booster on day 28. Serum was collected on days 21, 42 and 50 post priming.

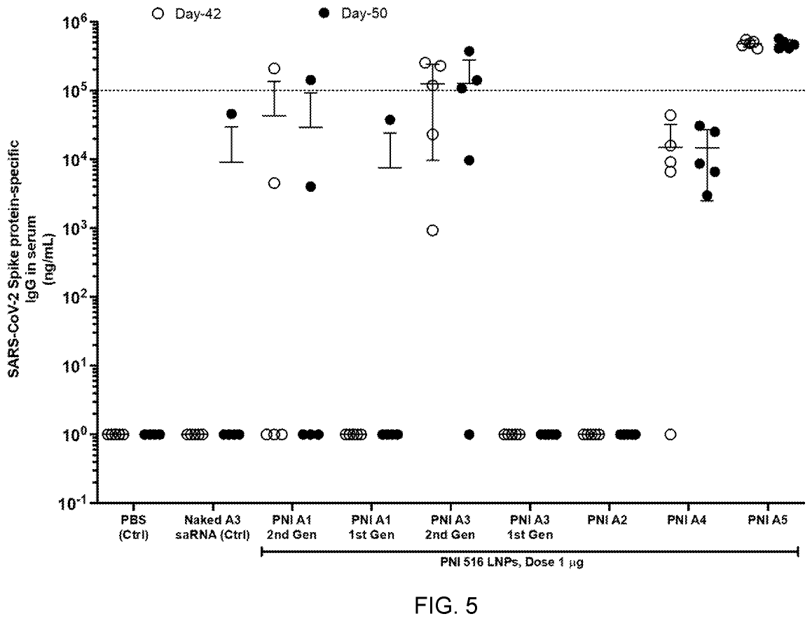

The IgG levels of mice independently inoculated with vectors NCOV PNI V101 encoding the genes of interest were measured, and the results were plotted. There were five mice per group, and the values were logarithmic. FIG. 5 shows the expression of anti-SARS-CoV-2 spike protein specific IgG levels following treatment with vectors Ncov PNI V101 independently encoding the different mRNA nCOV spike protein antigen designs in LNP. Measurements were taken on day 42 and day 50 serum samples using ELISA.

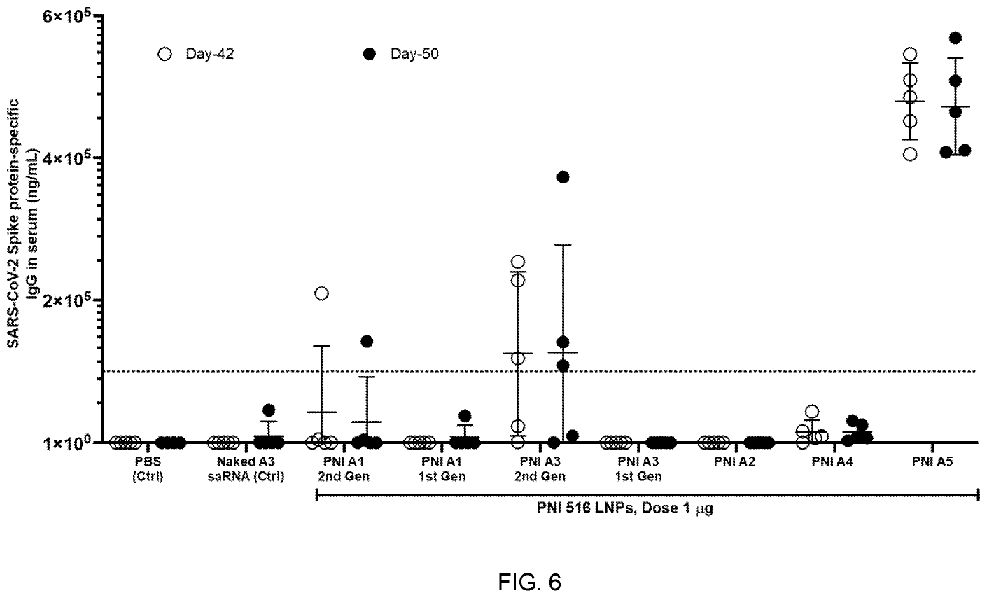

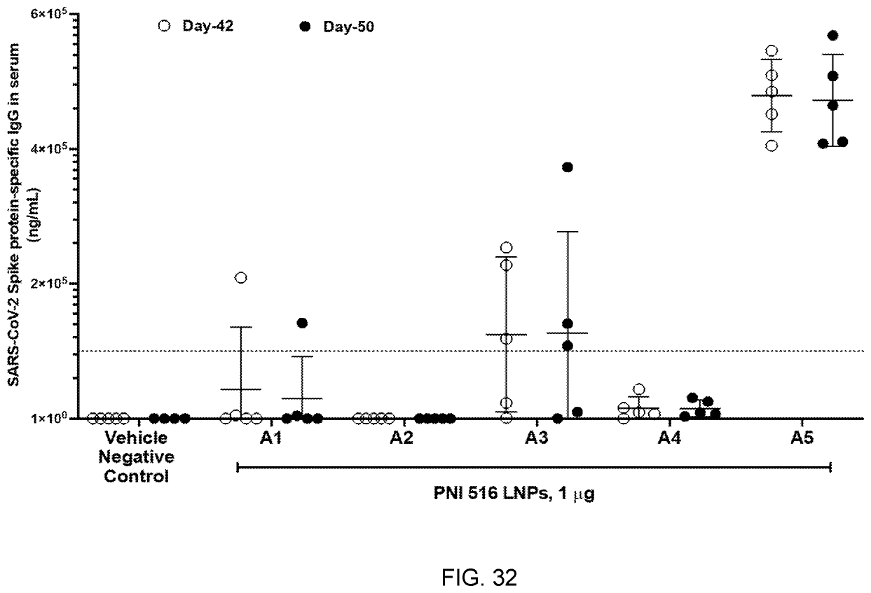

In a similar study, other controls were used, and the spike specific IgG were measured. In this case, naked PNI A3 was included as a control. Results are shown in FIG. 6. In both FIG. 5 and FIGS. 6, A3 and A5 were the best performers. Further data is presented in FIG. 32.

In conclusion: [PNI V101 nCOV A3 and A5 saRNA in VACCMIX-B PNI 516 LNP]-treated animals appeared to elicit strong anti-SARS-COV-2 spike protein specific IgG in a mouse model. Surprisingly, similar antigenic sequences did not perform as well. This suggests that only particular Spike protein saRNA driven antigenic sequences will be effective vaccine components.

### Example 7

This example demonstrates a pseudovirion neutralization assay.

A pseudovirion is a control vaccine sequence in which the test antigen sequence is embedded without the extra supports of the replicon or envelope protein and spike protein components. This tests the antigen in the absence of the other components of the specific virus, and is another measure of the efficacy of the antigen.

Mice were inoculated with vector nCOV PNI V101 pseudovirion vaccines. After 50 days, the mice were sedated and terminally exsanguinated. Blood was centrifuged down and sera diluted 2-fold serially, at 50 μl volume per well, on a 96 well plate. Pseudovirus psV-SARS-COV-2 was added to each well at TCID₅₀ at 50 μL per well and incubated for 1 h at 37 deg. C. Then, cell lines Vero E6 cells were added at 100 μL per well.

Plates were incubated again at 37 Deg. C for 72 h. After this, the supernatant was removed, and 50 μl beetroot juice lysis buffer was added to each well.

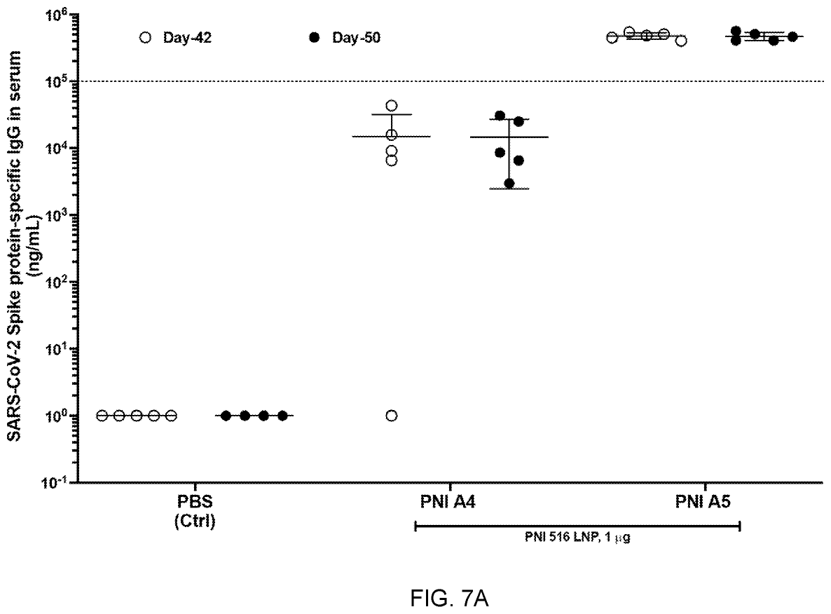

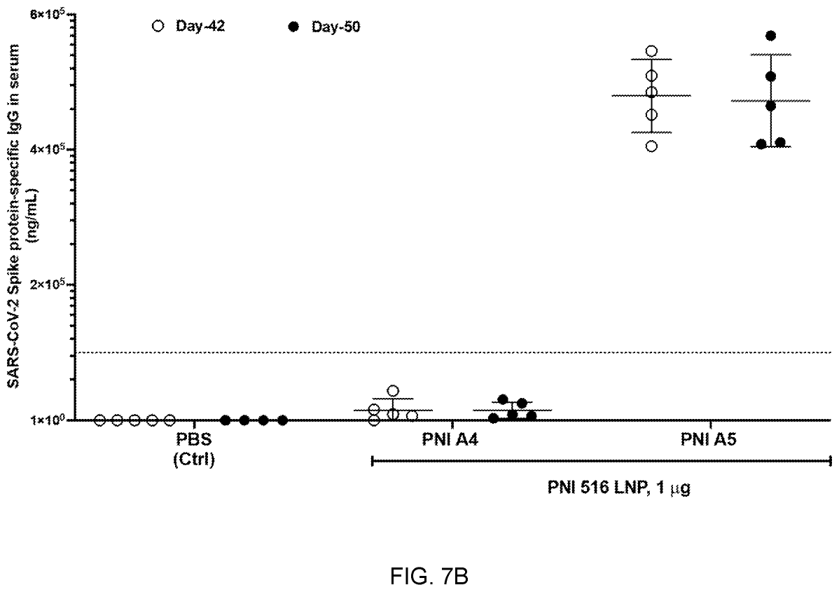

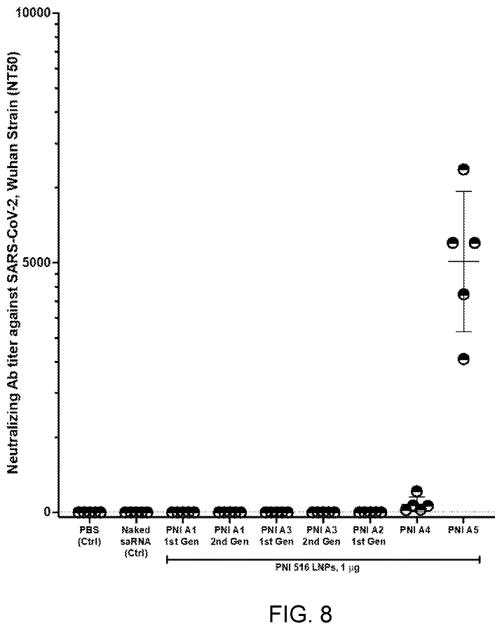

Luminescence was measured on a plate reader, and the results were collected and analyzed. Results of the studies are shown in FIGS. 7A-7B and FIG. 8. The results support the complete saRNA vaccine findings for PNI A5.

### Example 8

This example demonstrates the neutralizing antibody titer against SARS-COV-2, Wuhan Strain virus after administration of the vector of Example 1 to mice.

In another study of five animals inoculated with Gen PNI V101 nCOV (PNI A5) saRNA PNI 516 LNP, neutralizing antibody titer against SARS-COV-2, Wuhan Strain virus was assayed.

Mice were inoculated with vector nCOV PNI V101 vaccines, and after 50 days, sedated and terminally exsanguinated. Blood was centrifuged down, and sera was diluted 2-fold serially, at 50 μl volume per well, on a 96 well plate. Pseudovirus psV-SARS-COV-2 was added to each well at TCID₅₀ at 50 μL per well and incubated for 1 h at 37 deg. C., then cell lines Vero E6 cells were added at 100 μL per well.

Plates were incubated again at 37 Deg. C for 72h, after which supernatant was removed, and 50 μl beetroot juice lysis buffer was added to each well.

Luminescence was measured on a plate reader, and the results were collected and analyzed. Results of the studies are shown in FIGS. 7A-7B and FIG. 8. The results support the complete saRNA vaccine findings for PNI A5.

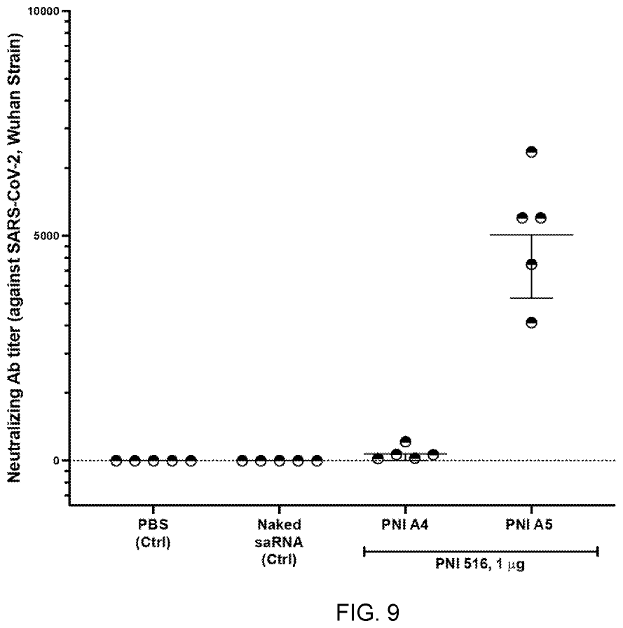

The results, shown in in FIG. 9, were statistically significant at p<0.0001, two-way ANOVA. The PNI V101 nCOV PNI A5 saRNA PNI 516 LNP elicited excellent humoral immune response in mice (SARS COV2 specific IgG in serum) compared to other nCOV saRNA PNI 516 LNP candidates and induced high neutralization antibody titer against original SARS-COV-2 (Wuhan) virus strain.

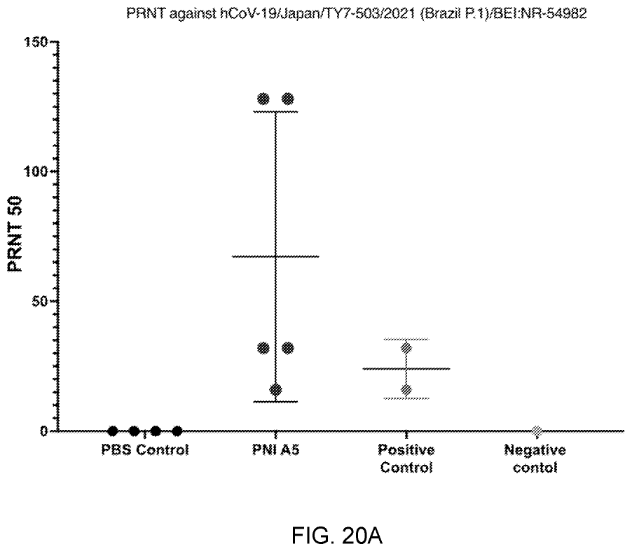

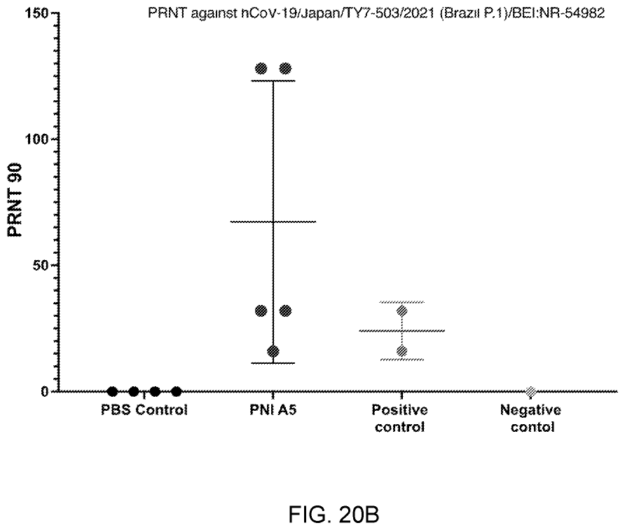

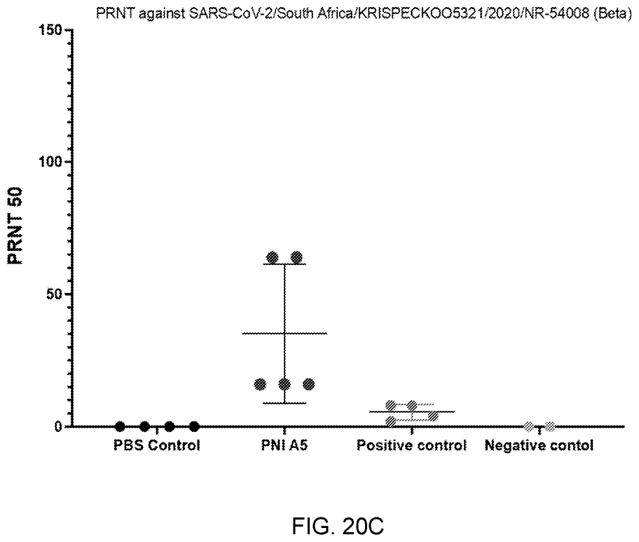

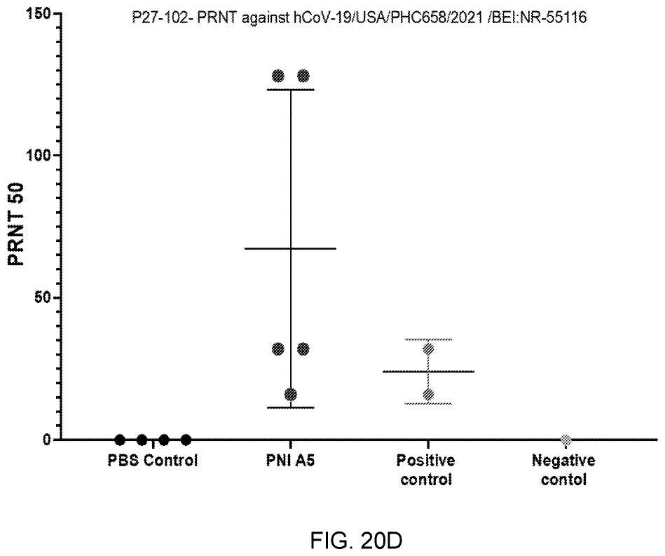

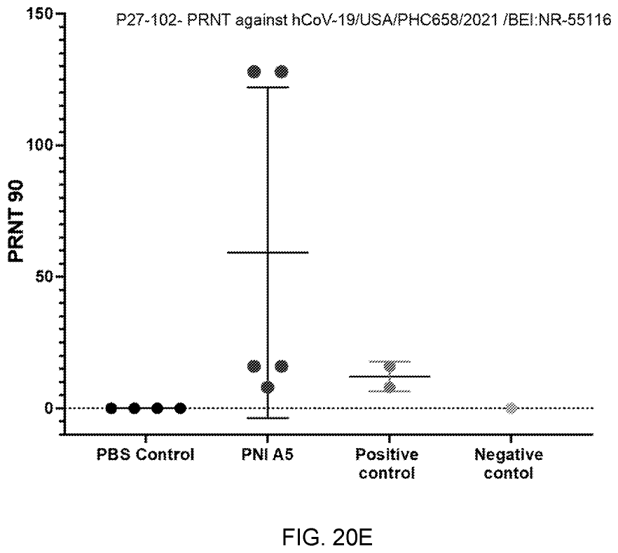

Variants were also tested. In plaque reduction neutralization tests (PRNT), Alpha variant hCov-19/Japan/TY7-503/2021 (BRAZIL P.1)/BEI/NR-54982, PNI V101 nCov A5 PNI 516 LNP excelled against positive control (convalescent human sera) and negative control (uninfected mouse sera). Results are shown in FIGS. 20A and 20B. Similar tests against the Beta variant strain SARS Cov2 South Africa/KRISPECKO05321/2020/NR-54008 (Beta) were also excellent as compared to a control. Result for the Beta variant is shown in FIG. 20C. As for the Delta variant strain hCov19 USA/PHC658/2021 BEI/NR 55116, the A5 antigen type was also better than controls. Results are shown in FIGS. 20D and 20E. The concentration of serum to reduce the number of plaques by 50% compared to the serum free virus gives the measure of how much antibody is present or how effective it is. This measurement is denoted as the PRNT50 value. The dilution which gives 90% reduction in the plaques compared to control is the PRNT90.

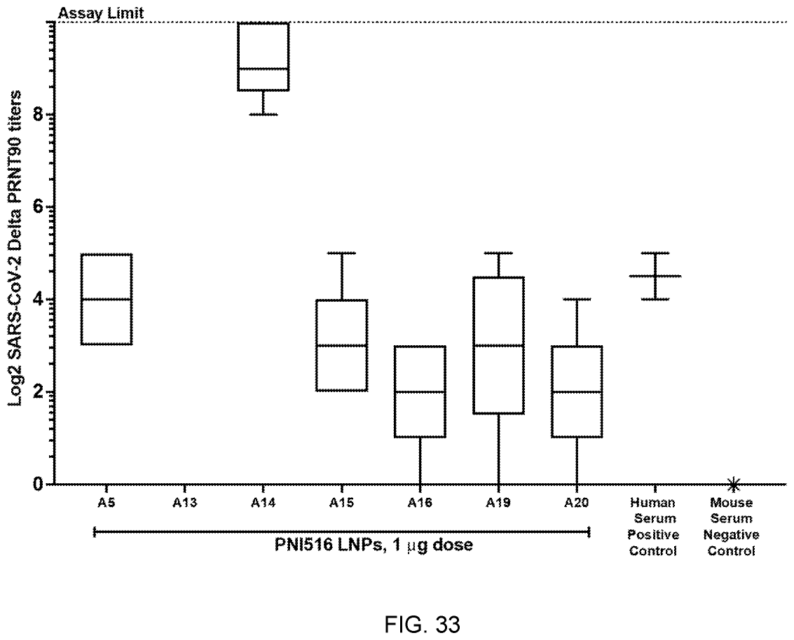

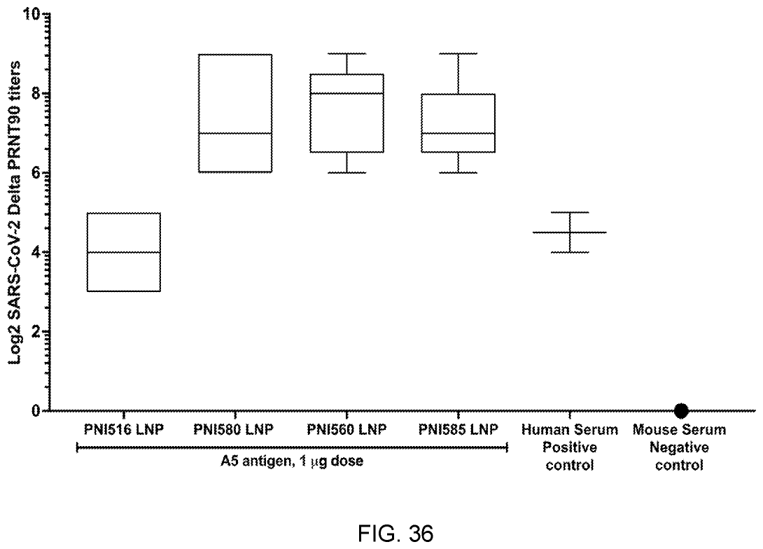

Additional neutralization data for the SARS-COV-2 Delta variant strain is presented in FIGS. 33 and 36.

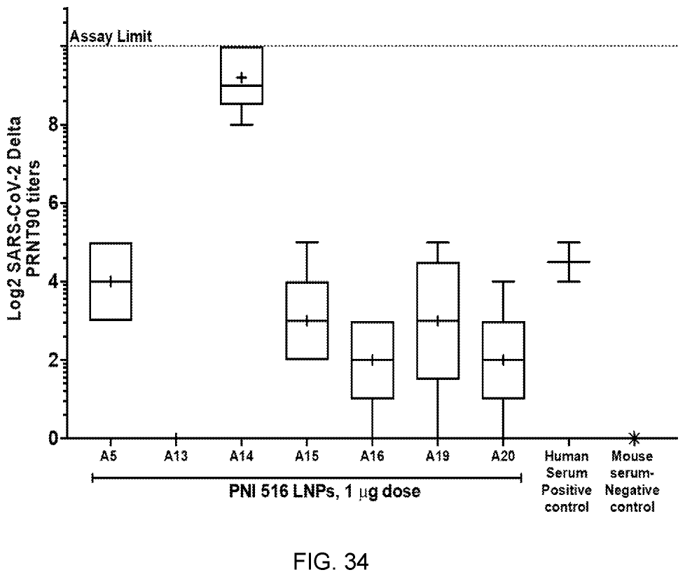

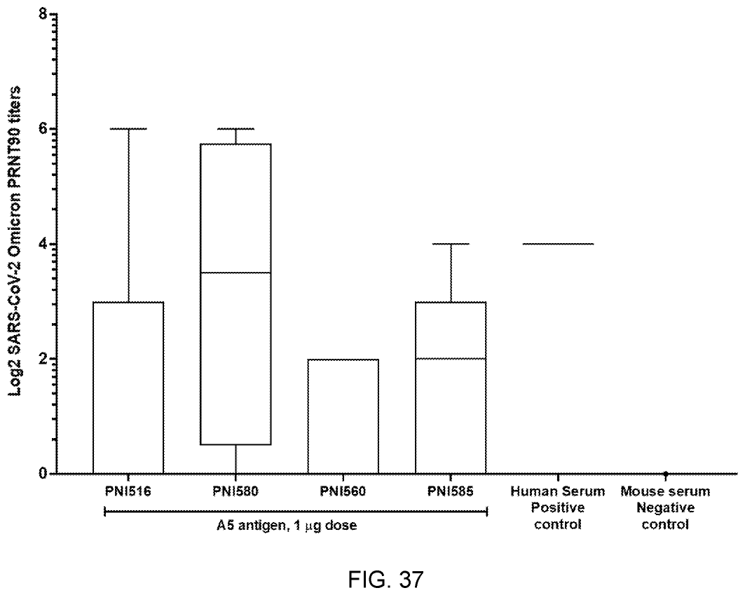

Additional neutralization data for the SARS-COV-2 Omicron variant strain is presented in FIGS. 34 and 37.

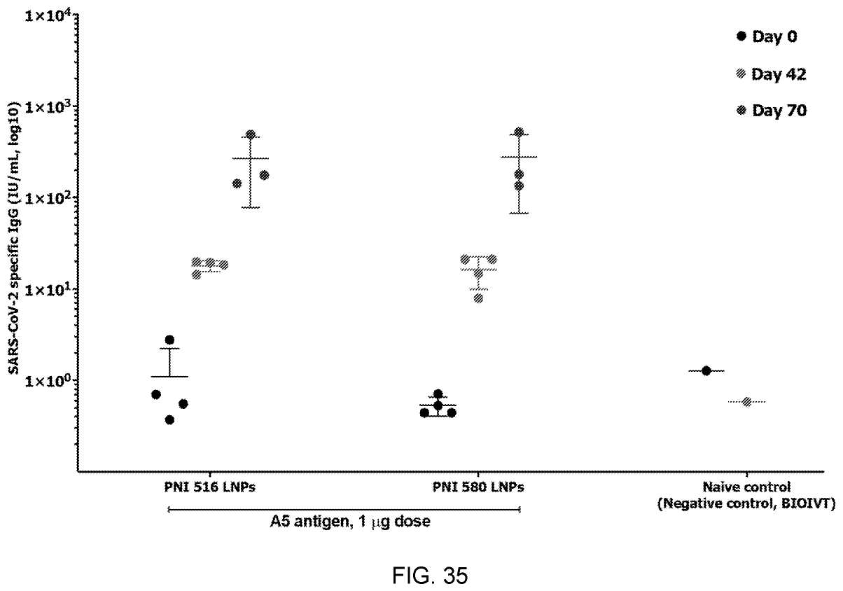

The expression of SARS-COV-2 specific IgG titer was also tested in Non-human Primates (NHPs) treated with PNI A5 antigen encapsulated in lipid nanoparticles from two different ionizable lipid compositions (FIG. 35).

### Example 9

This example demonstrates the impact of inoculation with the LNP of Example 2 on the T cells of mice.

Sera of mice that had been inoculated with the various antigen forms in LNP (Table 28) with were processed to isolate T cells.

SARS-COV2 Spike protein specific IgG production is a direct test to check the efficacy of the vaccine in vivo. A direct Enzyme Linked Immunosorbent assay (ELISA) was used to detect the efficiency of the lipid nanoparticle (LNP) based vaccine. Mice were given two intramuscular administrations of the vaccine with a specified time interval and the serum collected two weeks after the second dose (booster). Sera were serially diluted (1:100,000) to achieve desired concentration to detect within the limit of the detection (LOD) of the assay. In principle, a recombinant SARS COV2 Spike protein was used to coat on an ELISA plate, which was then exposed to the diluted serum. Antibodies generated in mice against the SARS COV2 Spike protein after the vaccination would be bound to the coated protein. A well-documented neutralizing mouse monoclonal antibody against SARS COV2 spike protein was used as the standard for the assay. All of the sera were quantified and tested against varying concentrations of the standard antibody using an anti-mouse IgG HRP. The antibody-ligand interaction was specifically detected by using a well-defined colorimetric technique based on HRP conversion of a colorless TMB substrate to a blue color solution. This time dependent color formation was further stopped by an acid solution and detected on a spectrophotometer at 450 nm. Readings in optical density (OD) were converted to an excel file and each data point is quantified using a slope created out of the standard.

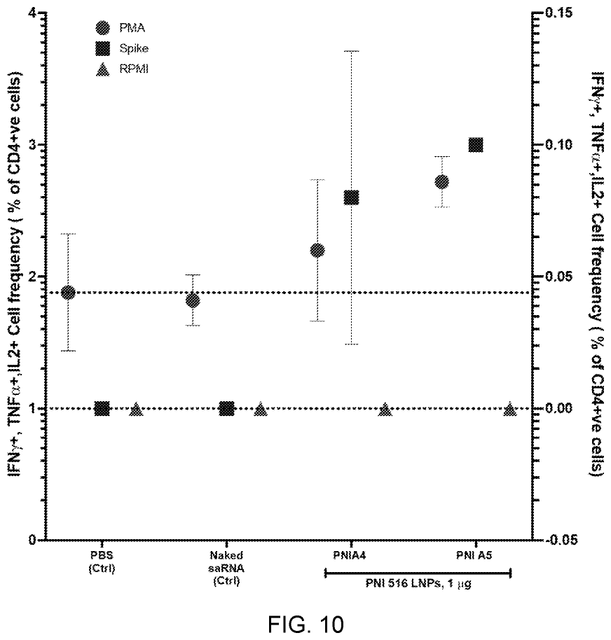

The impact of inoculation on CD4+ve or CD8+ve T cells in mice was examined. CD4+ve T cells as a percentage of live cells (T cells) specific for SARS-COV-2, Wuhan Strain virus, is shown in FIG. 10.

TABLE 28
Group Identifiers.

| | |
|---|---|
| Group 1 | PBS Control |
| Group 2 | Naked saRNA Ctrl |
| Group 3 | A1 (2^{nd} Gen) |
| Group 4 | A1 (1st Gen) |
| Group 5 | A3 (2^{nd} Gen) |
| Group 6 | A3 (1st Gen) |
| Group 7 | A2 |
| Group 8 | A4 |
| Group 9 | A5 |

CD4+IFN gamma+ve cell frequency as a percentage of live cells is shown in FIG. 10. CD4+TNF alpha+ve cell frequency as a percentage of live cells is shown in FIG. 10, and CD4+IFN gamma+ve TNF alpha+ve cell frequency as a percentage of live cells were measured and is shown in FIG. 10. Polyfunctional CD4+ve T cell response supports viral clearance and facilitates antiviral CD8+ve. Second Gen PNI V101 nCOV (PNI A5) saRNA PNI 516 LNP treated animals appear to elicit polyfunctional CD4+ve T cell response against the original SARS-COV-2 (Wuhan strain) virus. The PMA, spike protein and RPMI expression for different vaccination conditions is shown in Table 29.

TABLE 29
PMA, Spike Protein and RPMI expression
for different Vaccination conditions

| | PBS | Naked saRNA | PNI A4 PNI | PNI A5 PNI |
|---|---|---|---|---|
| | (Ctrl) | (Ctrl) | 516 LNP | 516 LNP |
| PMA | 1.88 ± 0.44 | 1.82 ± 0.192 | 2.2 ± 0.539 | 2.72 ± 0.192 |
| Spike | 0 | 0 | 0.08 ± 0.045 | 0.1 ± 0.00 |
| RPMI | 0 | 0 | 0 | 0 |

N = 5

### Example 10

This example demonstrates the CD8+ve T cell response against SARS-COV-2, Wuhan Strain virus in mice vaccinated with the LNP of Example 2.

In an in vitro study on the effects of the vaccine candidates on CD8+ve T cells with respect to CD8+percentage alone, IFN gamma positivity alone, TNF alpha positivity alone, IFN gamma positivity and TNF alpha positivity together, and IFN gamma positivity, TNF alpha positivity, and IL2+positivity together were measured. Polyfunctional CD8+T cells with IFN gamma, TNF alpha, and IL2+ levels were measured.

TABLE 30
Polyfunctional CD8+ve T cell (Cytotoxic T-cell) response facilitates
destruction of viral infected cells (Wuhan Strain Virus)

| | | Naked | PNI A4 | PNI A5 | PNI Lead |
|---|---|---|---|---|---|
| | PBS | saRNA | PNI 516 | PNI 516 | PNI 516 |
| | (Ctrl) | (Ctrl) | LNP | LNP | LNP |
| PMA | 4.62 ± | 4.3 ± | 5.06 ± | 6.76 ± | 7.1 ± |
| | 1.145 | 0.342 | 0.581 | 0.835 | 1.084 |
| Spike | 0 | 0 | 0.22 ± | 1.04 ± | 0.62 ± |
| | | | 0.084 | 0.241 | 0.259 |
| RPMI | 0 | 0 | 0 | 0 | 0 |

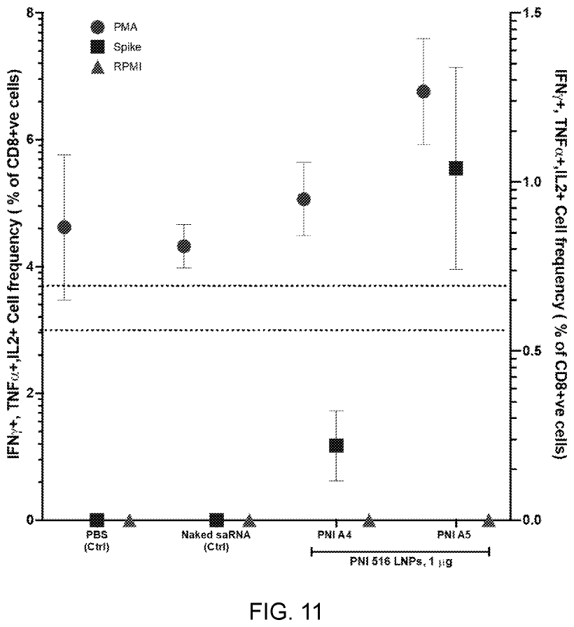

FIG. 11 shows the measured percentages of IFN gamma positivity, TNF alpha positivity, and IL2+positive CD8+ve T cells under the conditions of Table 30. Both 2nd Generation PNI V101 nCOV (PNI A5) saRNA PNI 516 LNP and PNI lead saRNA PNI 516 LNPs treated animals appear to elicit polyfunctional CD8+ve T cell response against the original SARS-COV-2 (Wuhan strain) virus.

Both CD4+ve and CD8+ve T cell immune responses against original SARS-CoV-2 (Wuhan) virus strain were observed in 2nd Generation PNI V101 nCOV (PNI A5) saRNA PNI 516 LNP.

### Example 11

This example demonstrates the effects of the ionizable lipid on the LNPs of Example 2.

A number of different ionizable lipids were tested in place of PNI 516 at NP 6 and NP 8 to see what effect the change had on potency and other characteristics.

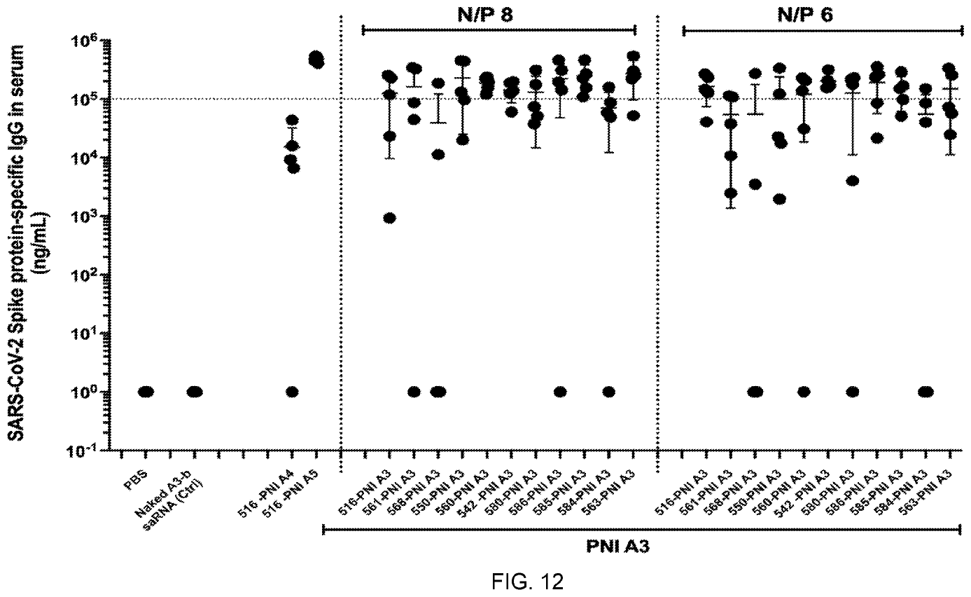

The potency of LNPs comprising different ionizable lipid structures was tested. The LNPs comprised nCov PNI V101 Self-amplifying mRNA encoding PNI A3 antigen. VACCMIX-A and VACCMIX-B (see Table 27) were the best formulation ratios tested (results of less effective formulations not shown). Serum anti-SARS-COV-2 spike protein specific IgG levels in inoculated mice are shown in FIG. 12 for formulations made with PNI 516, PNI 541, PNI 568, PNI 550, PNI 560, PNI 542, PNI 580, PNI 586, PNI 585, PNI 584, and PNI 563 at either N/P 8 or N/P 6 nitrogen to phosphate ratios. Structures for these compounds are disclosed in PCT Publications WO20252589 or WO21000041. Each animal's sera is represented by a black circle. Controls include naked A3 saRNA and a negative control. A graph of the results is shown in FIG. 12. A variety of ionizable lipids were effective for A3 nCOV-PNI vaccine sera. A3 activity was enhanced with different ionizable lipids as shown in FIG. 12.

As in other cases, the N/P 6 samples produced larger particles. The EE typically ranged from 86% to 99%. The N/P 6 ratio samples had a slightly lower encapsulation efficiency. These results are shown in FIGS. 13A and 13B.

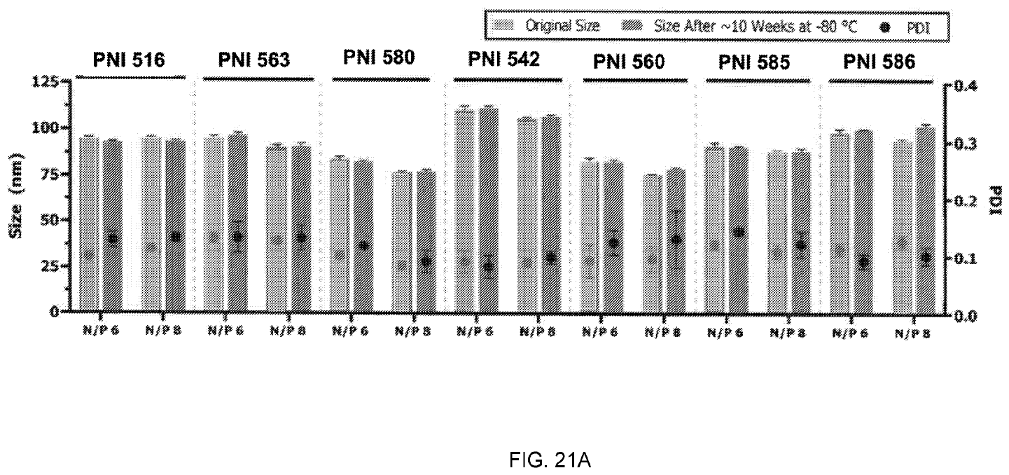

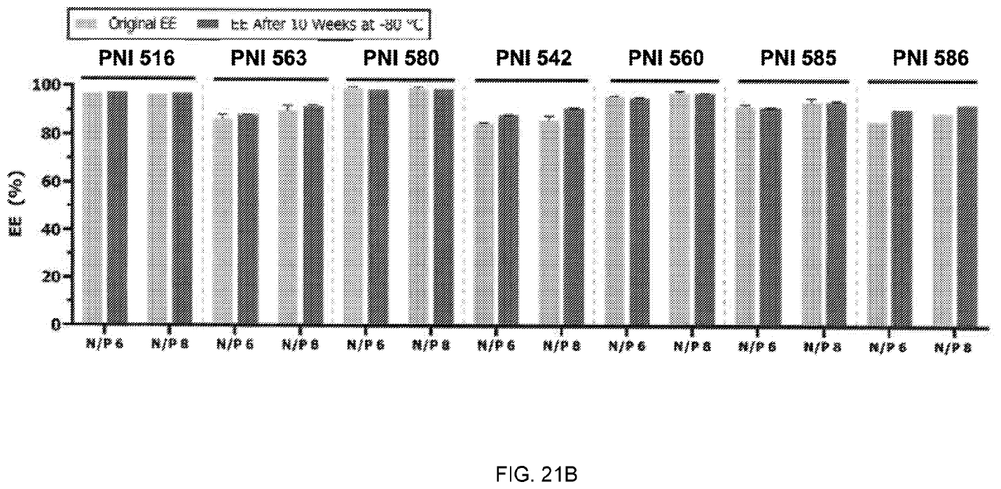

No significant changes in particle size or PDI were observed after 10 weeks of storage at about −80° C. (FIG. 21A). No significant changes in encapsulation efficiency were observed after 10 weeks of storage at about −80° C. (FIG. 21B).

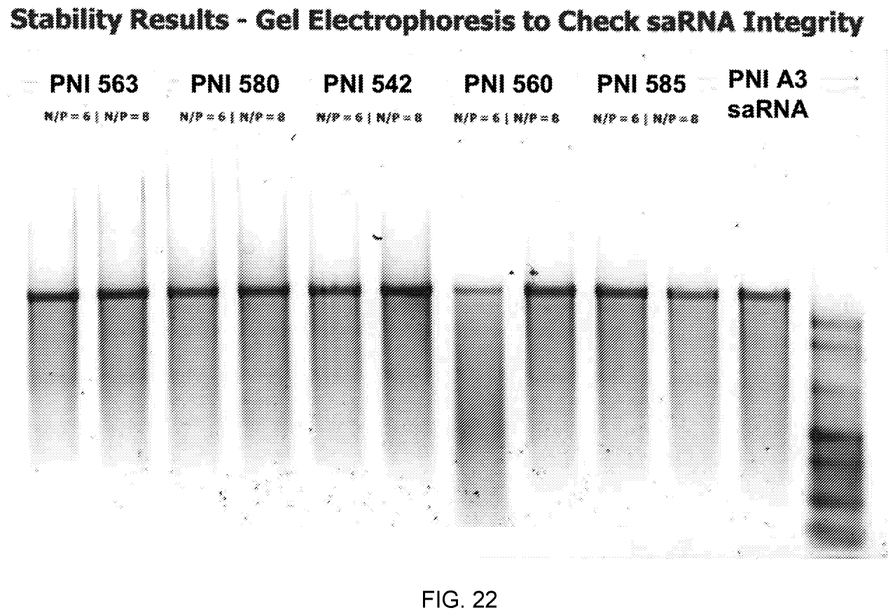

The samples were thawed after about 10 weeks of storage at −80° C. The integrity of saRNA was tested by gel electrophoresis. The results are shown in FIG. 22. For most samples, the saRNA band was comparable to naked A3.

### Example 12

This example demonstrates the in-vivo screening of PNI antigens, ionizable lipids and N/P ratio of the LNP of Example 2.

In a Western blot examination of in vitro expression, 2 μg/mL PNI antigens in the VACCMIX-A vector, equal or better spike protein expression was seen for N/P 6 v N/P 8 for most of the ionizable lipids (Table 31).

TABLE 31
Part 1, screening of antigens

| | | | |
|---|---|---|---|
| Group | Lipid | Payload | N/P (Lipid Mix) |

| | |
|---|---|
| 1 | 1X PBS Control |

| | | | |
|---|---|---|---|
| 2 | — | A3 | — |
| 3 | PNI 516 | 2^{nd} Gen A1 | 8 |
| 4 | | 1^{st} Gen A1 | (VACCMIX-A) |
| 5 | | 2^{nd} Gen A3 | |
| 6 | | 2^{nd} Gen A3 | |
| 7 | | A2 | |
| 8 | | A4 | |
| 9 | | A5 | |
| 10 | | ICL | |

### Example 13

This example demonstrates the spike protein IgG expression in mouse serum.

For Spike protein IgG expression in inoculated mice, particle size ranged from 70-95 nm, and PDI <0.115. The 2nd Generation saRNA-encapsulated LNPs were slightly larger, with an encapsulation efficiency (EE) of 96.5-99%. All of the LNPs were within an acceptable size, PDI range, and EE. The overall result was that A5 outperformed the other antigens (Table 32).

TABLE 32
Part 2: Ionizable Lipid and N/P Ratio Screening

| | | | N/P |
|---|---|---|---|
| Group | Lipid | Payload | (Lipid Mix) |
| 5 | PNI 516 | A3 | 8 |
| 11 | PNI 541 | | (VACCMIX-A) |
| 12 | PNI 568 | | |
| 13 | PNI 550 | | |
| 14 | PNI 560 | | |
| 15 | PNI 542 | | |
| 16 | PNI 580 | | |
| 17 | PNI 586 | | |
| 18 | PNI 585 | | |
| 19 | PNI 584 | | |
| 20 | PNI 563 | | |
| 21 | PNI 516 | A3-b | 6 |
| 22 | PNI 541 | | (VACCMIX-A) |
| 23 | PNI 568 | | |
| 24 | PNI 550 | | |
| 25 | PNI 560 | | |
| 26 | PNI 542 | | |
| 27 | PNI 580 | | |
| 28 | PNI 586 | | |
| 29 | PNI 585 | | |
| 30 | PNI 584 | | |
| 31 | PNI 563 | | |

### Example 14

This example demonstrates the efficacy of the eGFP PNI V101 replicon in an electroporation (one B18R variant) model.

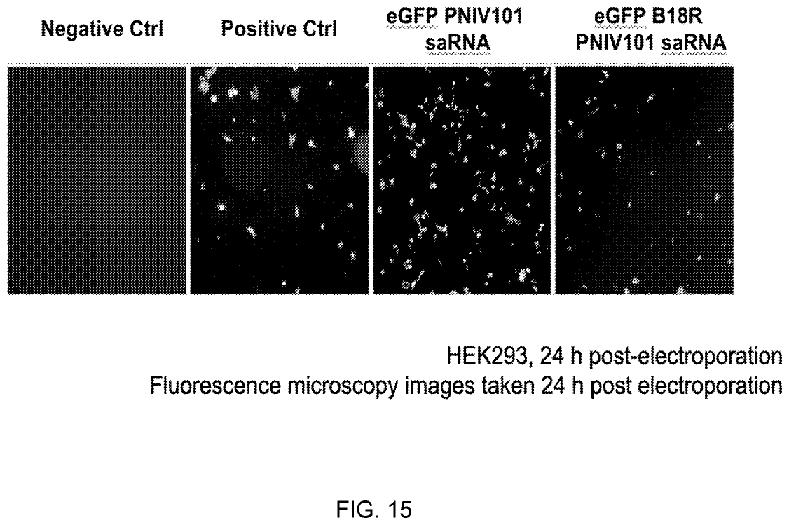

Two types of eGFP PNI V101 replicons were tested in HEK293 cells. Fluorescence microscopy was performed 24 hours post electroporation for both types. FIG. 15 shows the post electroporation results for control (no treatment), positive control (eGFP), the PNI V101 replicon, and the variant B18R replicon. The PNI V101 replicon was highly effective, giving more signal than control.

### Example 15

This example demonstrates the efficacy of the eGFP PNI V101 replicon.

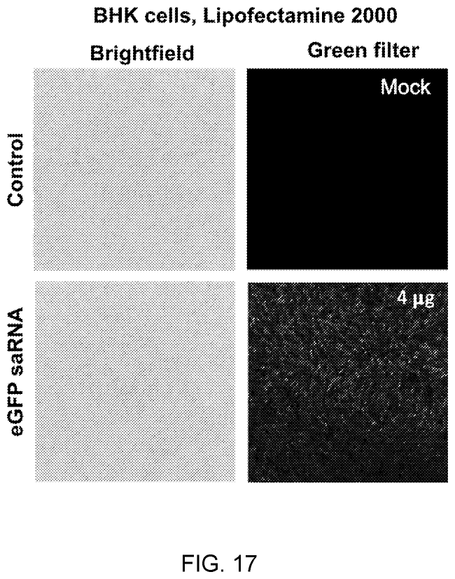

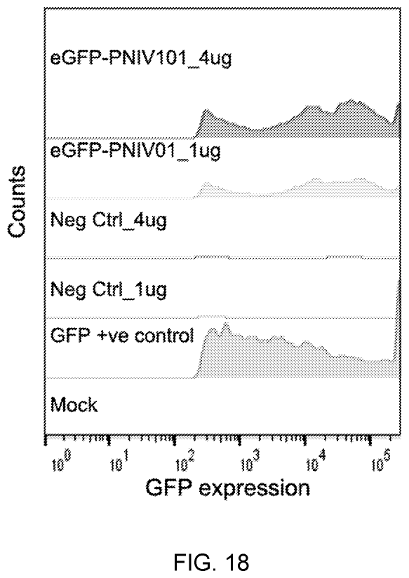

BHK cells were exposed to Lipofectamine-associated eGFP PN101. Fluorescence microscopy was performed with the green filter as well as bright field analysis. As shown in FIG. 17, the cells for both control and PNI V101 transfected tests looked healthy, and eGFP expression was good at a dose of 4 μg.

### Example 16

This example demonstrates the efficacy of the eGFP PNI V101 replicon.

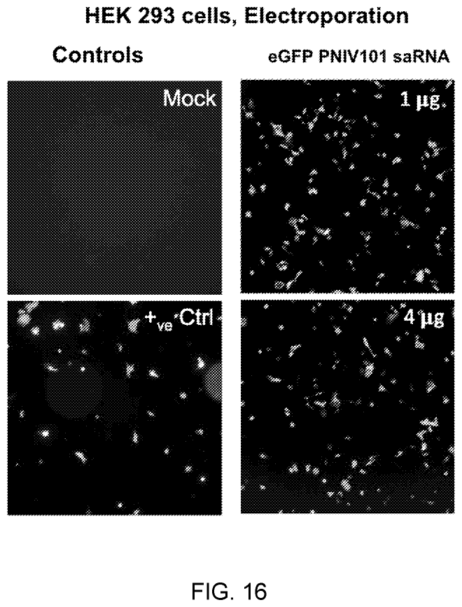

HEK293 cells in vitro were transfected with eGFP-PNI V101 at 4 and 1 μg, negative control at 4 and 1 μg, positive control GFP and mock transfection. MFI was measured per area and GFP expression is shown for each population. PNI V101 performed well at expressing GFP in vitro. Results are shown in FIG. 16 and in Table 33.

TABLE 33
EGFP-PNI V101 GFP Expression by MFI

| RNA | % GFP | MFI |
|---|---|---|
| eGFP-PNI V101 saRNA_4 μg | 54.1 | 12193 |
| eGFP-PNI V101 saRNA_1 μg | 27 | 12609 |
| Neg Ctrl saRNA_4 μg | 1.04 | 3088 |
| Neg Ctrl saRNA_1 μg | 0.51 | 1377 |
| GFPpDNA_+ve control | 82.3 | 5124 |
| Mock | 0.17 | 374 |

### Example 17

This example demonstrates the ability of PNI V101 replicon to deliver more than just SARS nCov-2 vaccine elements.

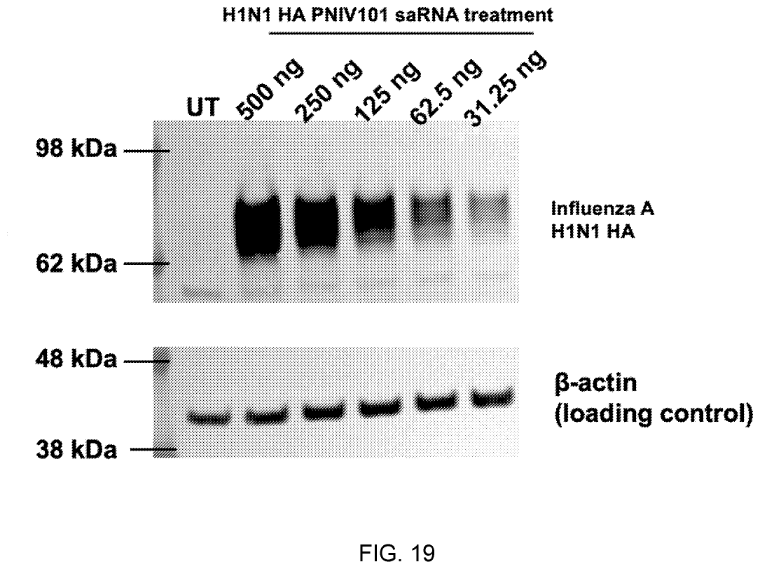

Influenza A H1N1 HA Polyclonal Antibody in WB (Cat #: PA5-34929, Invitrogen) was delivered to cells by the PNI V101 replicon. BHK-570 cells were treated in vitro with the test agents, then after 24 hours, processed and protein run on gel. Beta actin acted as control, and untreated as a negative control. PNI 516 VACCMIX-A LNP was the carrier. HA Expression was dose dependent. Results are shown in FIG. 19.

### Example 18

This example demonstrates the screening of ionizable and structural lipids in the LNPs.

The study groups for these experiments are shown in Table 34.

TABLE 34

| | Ionizable | Lipid Mix |
|---|---|---|
| Group | Lipid | Composition |
| 1 | PNI 516 | VACCMIX-A |
| 2 | | VACCMIX-A_DOPC |
| 3 | | VACCMIX-A_DPPC |
| 4 | PNI 585 | VACCMIX-A |
| 5 | | VACCMIX-A_DOPC |
| 6 | | VACCMIX-A_DPPC |
| 7 | PNI 560 | VACCMIX-A |
| 8 | | VACCMIX-A_DOPC |
| 9 | | VACCMIX-A_DPPC |
| 10 | PNI 542 | VACCMIX-A |
| 11 | | VACCMIX-A_DOPC |
| 12 | | VACCMIX-A_DPPC |
| 13 | PNI 580 | VACCMIX-A |
| 14 | | VACCMIX-A_DOPC |
| 15 | | VACCMIX-A_DPPC |
| 16 | PNI 563 | VACCMIX-A |
| 17 | | VACCMIX-A_DOPC |
| 18 | | VACCMIX-A_DPPC |
| 19 | PNI 586 | VACCMIX-A |
| 20 | | VACCMIX-A_DOPC |
| 21 | | VACCMIX-A_DPPC |

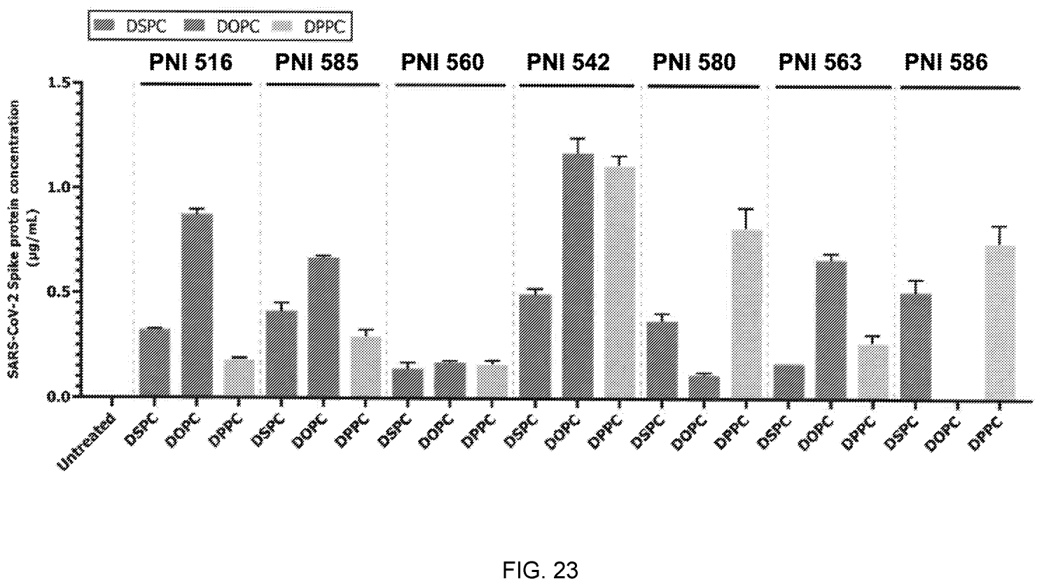

HEK 293 cells were treated with 0.25 μg/mL with the LNPs of Table 34. SARS-CoV-2 spike protein concentration was measured. The general trend was that the greatest protein concentrations were found in the LNPs with DOPC, the next greatest protein concentrations were found in the LNPs with DPPC, and the third greatest protein concentrations were found in the LNPs with DSPC (FIG. 23).

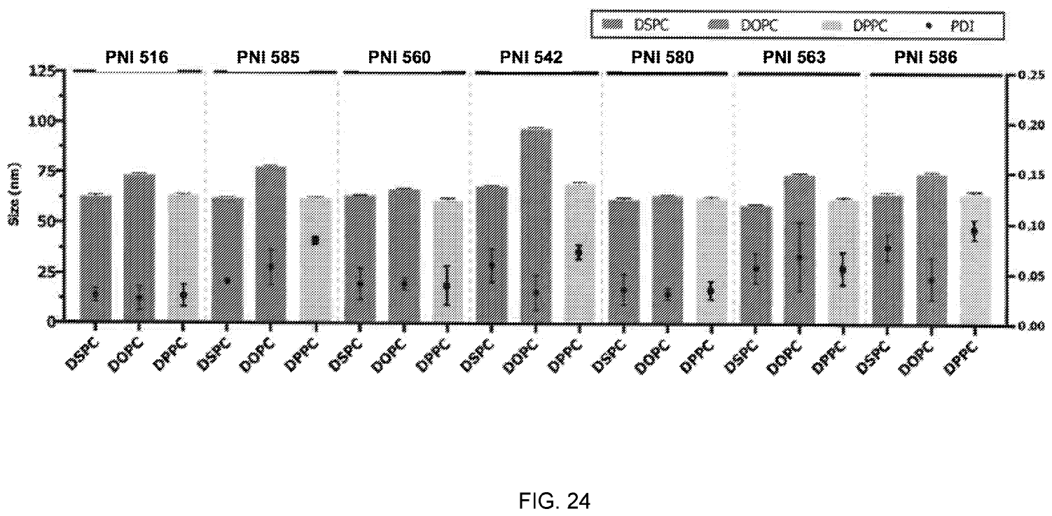

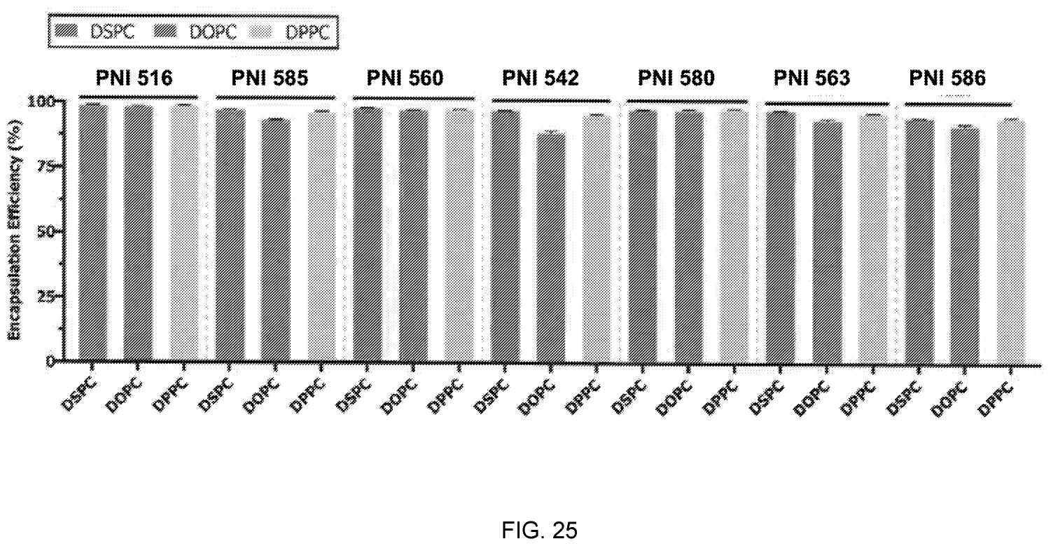

The results of sizing, PDI, and encapsulation efficiency (EE) for the LNPs of Table 34 are shown in FIG. 24 (size and PDI) and FIG. 25 (EE). The size range was determined to be from 60 to 100 nm. The PDI range was less than 0.1. LNPs with DOPC produced larger LNPs. The EE range was from 90 to 99%. The LNPs with DOPC provided slightly lower EE values.

### Example 19

This example demonstrates the stability of the sizes and PDI of an LNP.

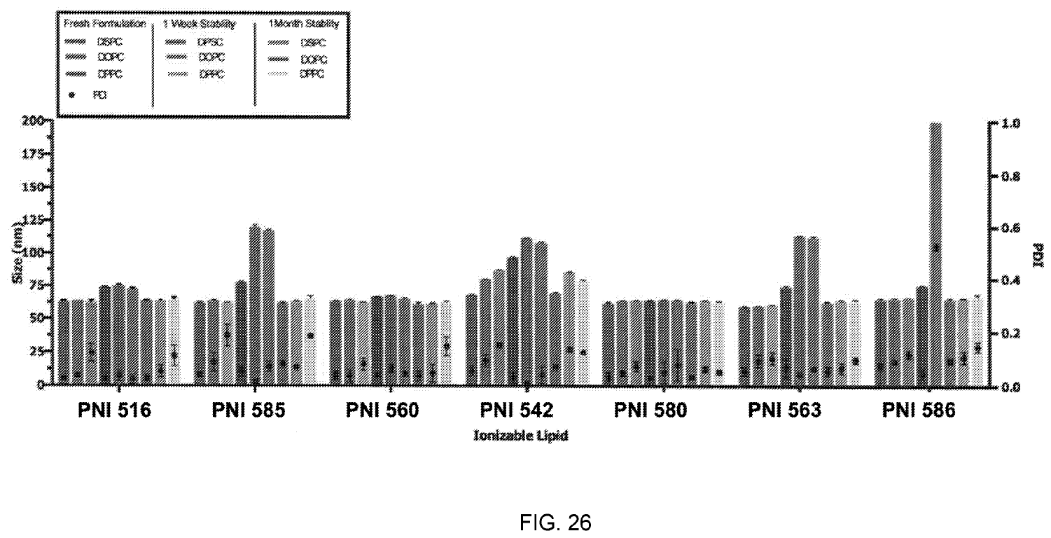

LNPs were prepared with various ionizable lipids and structural lipids according to the following general formula: 47.5% ionizable lipids, 38.5% cholesterol, 12.5% structural lipid, and 1.5% PEG-DMG. The payload was PNI A5. The structural lipid was DSPC, DOPC, or DPPC. The ionizable lipid was one of those described in Table 34. The stability of the size and PDI was measured after no storage time (fresh formulation), after a 1 week storage, and after a 1 month storage. The results are shown in FIG. 26.

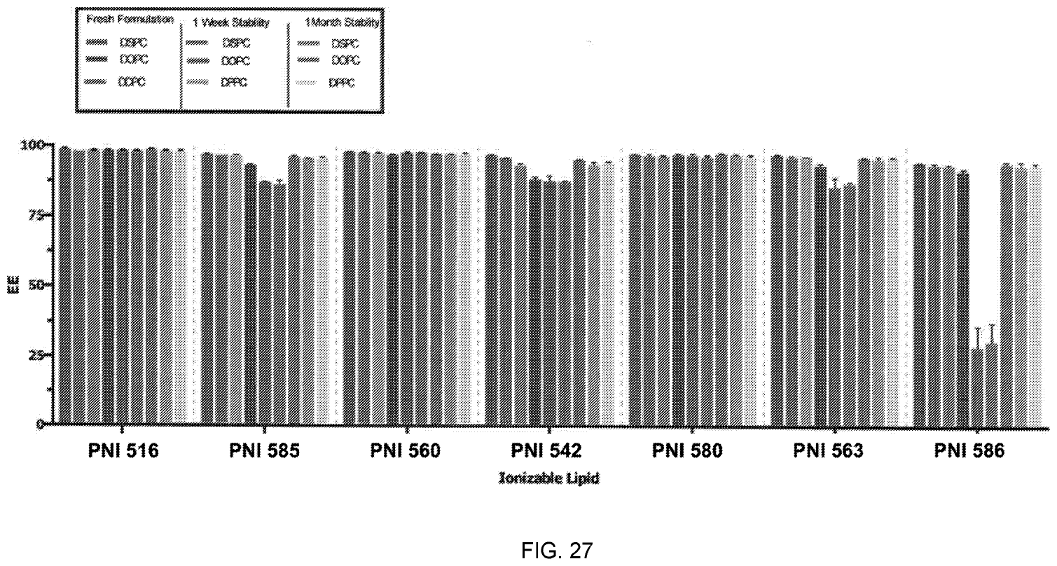

The stability of the encapsulation efficiency was measured after no storage time (fresh formulation), after a 1 week storage, and after a 1 month storage. The results are shown in FIG. 27.

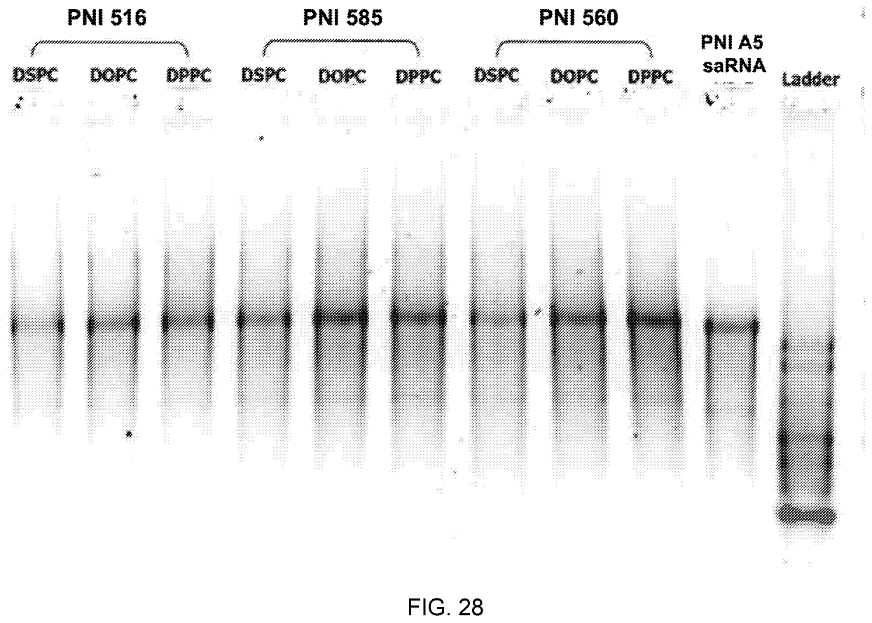

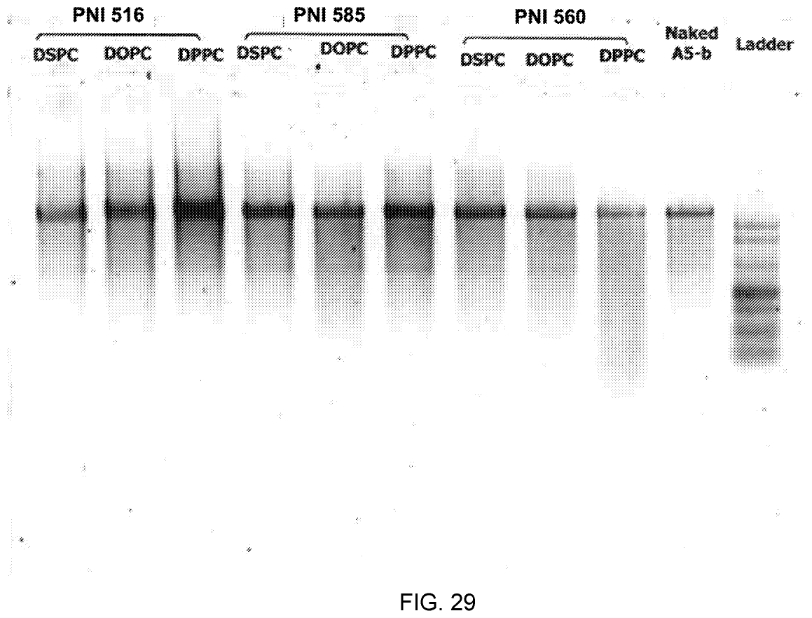

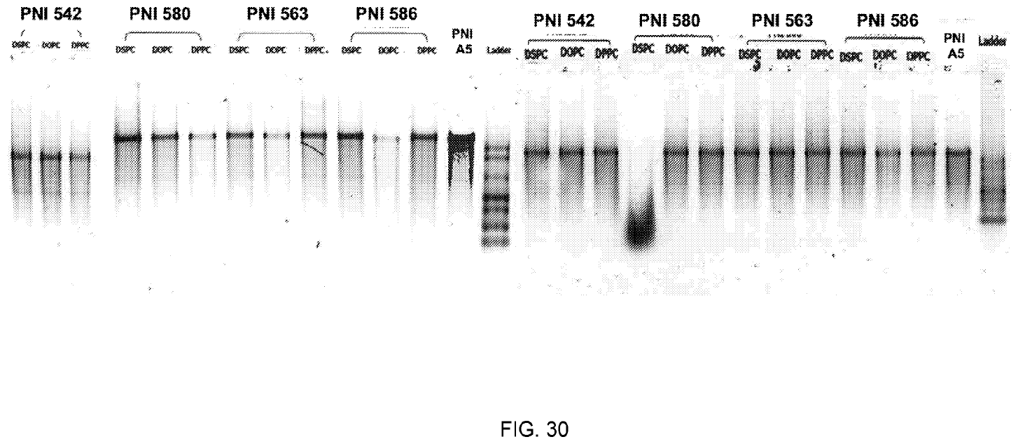

The amount of A5 antigen extracted from LNPs prepared with the various ionizable lipids and structural lipids was measured by gel electrophoresis following 1 week of storage (FIGS. 28 and 30) and following 1 month of storage (FIGS. 29-30).

The stability of the formulation PNI 541 LNPs was measured after 5 months of storage at 4° C. A graph showing the chromatogram of lipids following stability analysis of an LNP after 5 months of storage at 4° C. was generated and studied. The intact molecular signature of each lipid component of the LNPs such as sterol, DSPC, PEG-DMG and PNI 541 were observed.

### Example 20

This example demonstrates that neutralizing antibodies are raised against SARS-CoV-2 in BALB/c mice inoculated with nCOV saRNA LNPs.

The sera from mice inoculated with nCOV saRNA LNPs were used for a plaque reduction neutralization test (PRNT) or a pseudo-virion neutralization-based assay.

The neutralizing antibodies induced against severe acute respiratory syndrome coronavirus 2 isolate WA1 (SARS-COV-2/WA1/2020) (BEI: NR-52281) were assessed in 48 mice sera by plaque reduction neutralization test (PRNT). Two human sera collected from male individuals fully vaccinated with a SARS-COV-2 vaccine and a convalescent non primate human (NPH) serum were used as positive SARS-COV-2 Ab serum. A naïve mouse serum was used as negative SARS-COV-2 Ab serum.

Sera were incubated at 56° C. for 30 minutes. Serial two-fold dilution of each serum were prepared (1/2 to 1/64). Fifteen (15) μl of each dilution and fifteen (15) μl of SARS-COV-2 stock containing thirty (30) plaque forming unit (PFU) of virus were mixed and incubated at 37° C. for one (1) hour. The titer of SARS-COV-2 was determined by plaque assay on Vero E6 cells. The supernatants of monolayer of Vero E6 cells in 12-well plates were removed and two hundred (200) μl of double minimum essential eagle medium (DMEM) were added to each well. Then one hundred (100) μl DMEM was added to each thirty (30) μl mixture of virus and serum, and the total mixture of each well was added into monolayer Vero-E6 and incubated at 37° C. Plates were shacked each 10-15 minutes during incubation time. After one-hour incubation, 1.5 ml overlayer containing 2X DMEM and 0.6% agarose with ratio of 1:1 was added to each well and were incubated at 37° C. Seventy-two (72) hours after incubation, 0.5 ml of 10% formaldehyde were added into each well and plates were kept in room temperature under biosafety cabinet over night for fixation of cells. After fixation, 10% formaldehyde were collected and stored at a labeled bottle for formalin waste. The palettes of agarose were removed from wells and cells were stained by adding enough amount of 1% crystal violet into each well for 10-15 minutes. The crystal violet was collected and discarded into a waste container containing Bleach. Wells were washed with water and the plaques of SARS-COV-2 were counted for each well.

The titer was calculated based on 90% reduction in plaque counts (PRNT90). In this study, a PRNT90 titer was preferred over titers using lower cut-offs (PRNT50), because only thirty (30) PFU of virus was used, not one hundred (100) PFU, which is usually recommended for virus neutralization assays.

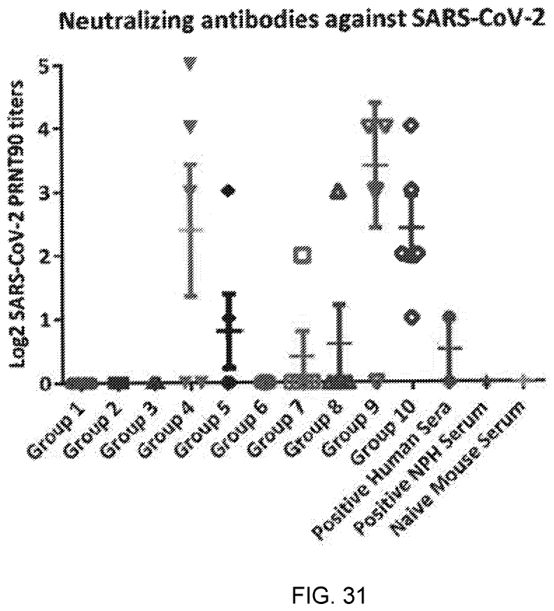

The titer of neutralizing antibodies against thirty (30) PFU of SARS-COV-2/WA1/2020 for each mouse serum is shown in FIG. 31 and Table 35. The highest dilution of serum that reduced the PFU of virus to equal or lower than three (3) PFU was considered as the titer of neutralizing antibodies against SARS-COV-2 based on PRNT90. The results showed that there was about thirty (30) PFU of virus for each dilution of naïve mouse serum, regardless the dilution of serum. Although high titers of neutralizing antibodies were not detected in positive control sera based on PRNT90, both human positive control sera and NPH positive control serum reduced the number of virus particles.

TABLE 35
PRNT Assay (The highest dilution of serum which reduced 90% of
the PFU of virus (3 or lower PFU) was considered as the titer
of neutralizing antibodies against SARS-CoV-2 based on PRNT90).

| | Serum Dilution | | | | | | |
|---|---|---|---|---|---|---|---|
| Mouse | 1/2 | 1/4 | 1/8 | 1/16 | 1/32 | 1/64 | PRNT90 |
| 1-1 | NC | NC | NC | 28 | 27 | 31 | 0 |
| 1-2 | NC | NC | NC | NC | NC | 27 | 0 |
| 1-3 | NC | NC | NC | NC | NC | 28 | 0 |
| 1-4 | NC | NC | NC | NC | 21 | 23 | 0 |
| 2-1 | 24 | 25 | 31 | 27 | 24 | 28 | 0 |
| 2-2 | NC | 27 | 25 | 25 | 24 | 23 | 0 |
| 2-3 | 26 | 22 | 21 | 24 | 26 | 23 | 0 |
| 2-4 | 18 | 20 | 21 | 18 | 21 | 20 | 0 |
| 2-5 | 24 | 20 | 29 | 25 | 32 | 22 | 0 |
| 3-1 | 25 | NC | 27 | 25 | 24 | 19 | 0 |
| 3-2 | 15 | 17 | 18 | 16 | 21 | 15 | 0 |
| 3-3 | 9 | 11 | 22 | 16 | 16 | 22 | 0 |
| 3-4 | 4 | 10 | 17 | 16 | 14 | 23 | 0 |
| 3-5 | 5 | 6 | 6 | 9 | 12 | 21 | 0 |
| 4-1 | 6 | 9 | 7 | 15 | 11 | 20 | 0 |
| 4-2 | 0 | 0 | 4 | 2 | 7 | 11 | 16 |
| 4-3 | 0 | 0 | 0 | 0 | 3 | 10 | 32 |
| 4-4 | 0 | 2 | 3 | 6 | 7 | 15 | 8 |
| 4-5 | 8 | 11 | 8 | 9 | 16 | 16 | 0 |
| 5-1 | 16 | 14 | 19 | 23 | 22 | 20 | 0 |
| 5-2 | 1 | 2 | 0 | 4 | 7 | 15 | 8 |
| 5-3 | 1 | 5 | 8 | 9 | 17 | 14 | 2 |
| 5-4 | 12 | NC | 9 | 18 | 18 | 17 | 0 |
| 5-5 | 10 | 12 | 10 | 8 | 19 | 18 | 0 |
| 6-1 | 4 | 2 | NC | NC | 21 | NC | 0 |
| 6-2 | 21 | 18 | NC | 22 | NC | NC | 0 |
| 6-3 | 10 | 9 | NC | 31 | 25 | 25 | 0 |
| 6-4 | NC | NC | 21 | NC | NC | 20 | 0 |
| 6-5 | NC | NC | NC | NC | 26 | 25 | 0 |
| 7-1 | 3 | 8 | 11 | 16 | 15 | 22 | 2 |
| 7-2 | 13 | 12 | 17 | 25 | 18 | 20 | 0 |
| 7-3 | 5 | 6 | 10 | 14 | 15 | 21 | 0 |
| 7-4 | 11 | 20 | 17 | 21 | 15 | 17 | 0 |
| 7-5 | 27 | 20 | 20 | 21 | 25 | 21 | 0 |
| 8-1 | 18 | 17 | 12 | 19 | 21 | 23 | 0 |

NC: non countable >30 particles

### Example 21

This example demonstrates the versatility of the customizable vector of SEQ NO: 1.

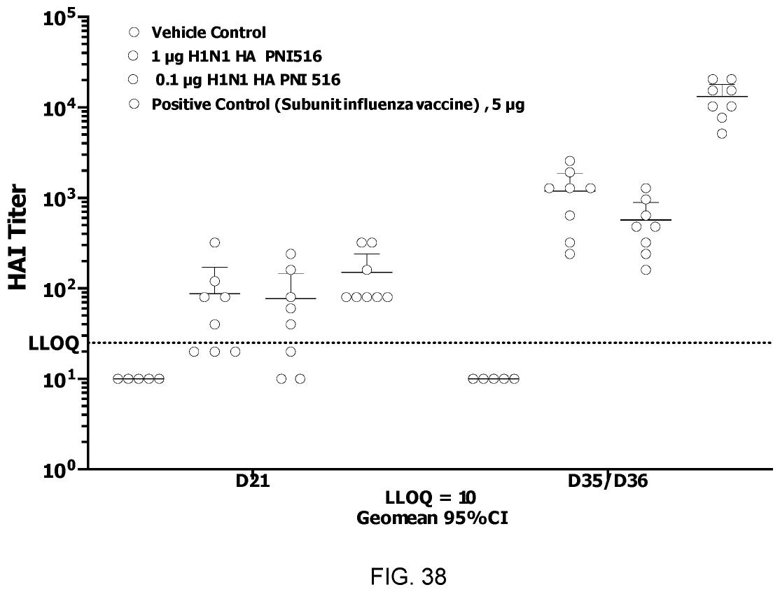

The influenza A/California/07/2009 (H1N1) Hemagglutinin Inhibition (HAI) titer levels were measured following treatment of 6-8 week-old female BALB/c mice with H1N1 HA saRNA encapsulated in PNI 516 LNPs. The results are shown in FIG. 38.

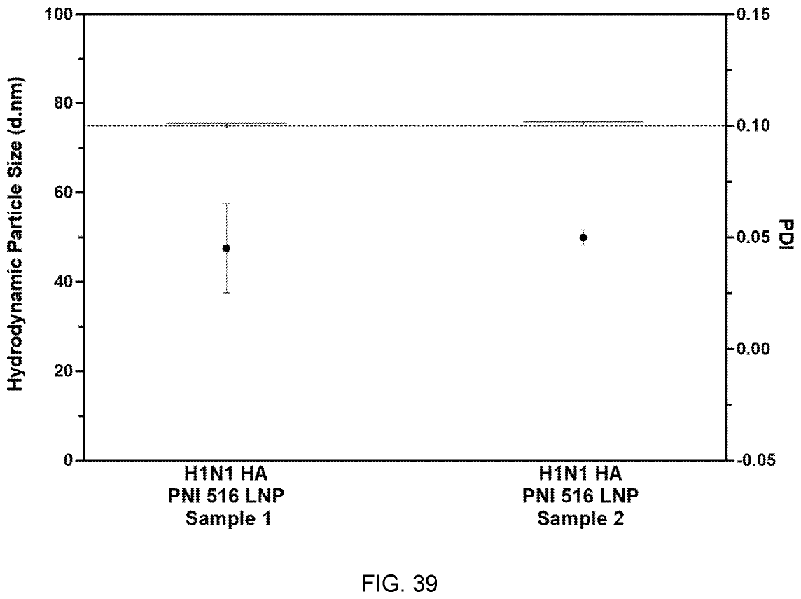

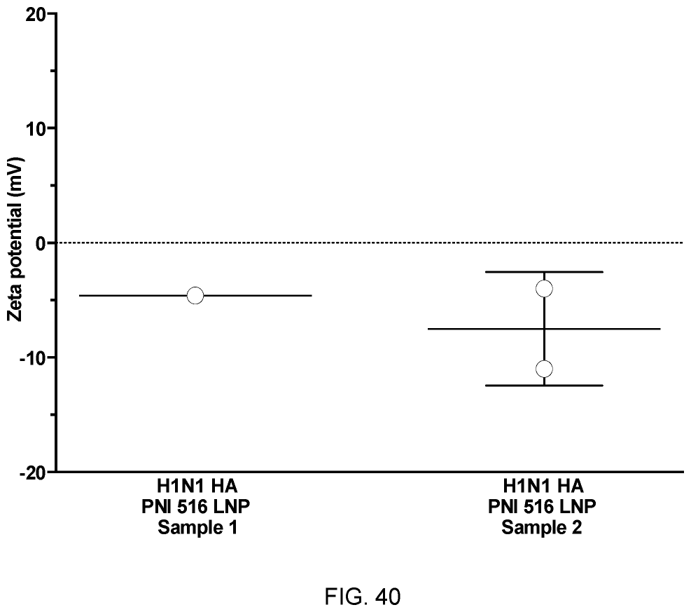

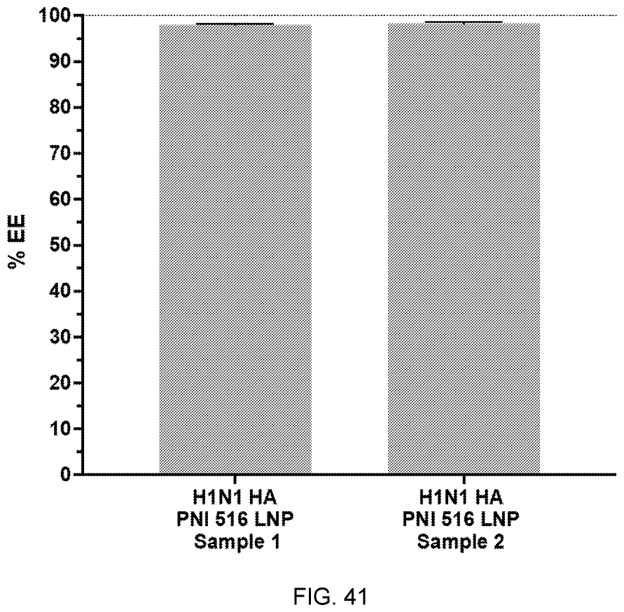

Tests of the physiochemical characteristics of Influenza A/California/07/2009 (H1N1) HA PNI 516 LNP samples showed that the hydrodynamic particle size was in the sub-100 nm range (FIG. 39). The Zeta potential of the particles were near neutral but mildly negatively charged (FIG. 40). The encapsulation efficiency (% EE) was greater than 90% (FIG. 41).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant expression vector comprising a nucleotide sequence comprising:
(a) a Venezuelan Equine Encephalitis Virus (VEEV) 5′ untranslated region (5′-UTR);
(b) a nucleotide sequence encoding VEEV non-structural proteins nsP1, nsP2, nsP3 and nsP4;
(c) a VEEV 26S sub-genomic promoter;
(d) an engineered multiple cloning site (MCS);
(e) a VEEV 3′ untranslated region (3′-UTR); and
(f) a nucleotide sequence encoding a VEEV poly A sequence.

2. The recombinant expression vector of claim 1, wherein the nucleotide sequence encoding the VEEV poly A sequence comprises from 38 to 40 base pairs.

3. The recombinant expression vector of claim 1, wherein the VEEV 26S sub-genomic promoter is a VEEV TC83 strain 26S sub-genomic promoter.

4. The recombinant expression vector of claim 1, wherein a gene of interest is insertable at the engineered MCS.

5. The recombinant expression vector of claim 1, wherein the recombinant expression vector comprises, in order from 5′ to 3′, the following components:
(a) the VEEV 5′ untranslated region (5′-UTR);
(b) the nucleotide sequence encoding the VEEV non-structural proteins nsP1, nsP2, nsP3 and nsP4;
(c) the VEEV 26S sub-genomic promoter;
(d) the engineered MCS;
(e) the VEEV 3′ untranslated region (3′-UTR); and
(f) the nucleotide sequence encoding a VEEV poly A sequence.

6. The recombinant expression vector of claim 1, wherein the MCS is positioned directly adjacent to the 5′ end or the 3′ end of the nucleotide sequence encoding the VEEV poly A sequence.

7. The recombinant expression vector of claim 1, comprising a vector backbone comprising one or more of ColE, an origin of replication (ori), a tet promoter, and one or more antibiotic resistance genes.

8. The recombinant expression vector of claim 1, comprising a bacterial vector backbone or a modified bacterial vector backbone.

9. The recombinant expression vector of claim 1, comprising a T7 promoter adjacent to the 5′ end of the 5′ UTR.

10. The recombinant expression vector of claim 1, comprising a nucleotide sequence at least 85% identical to SEQ ID NO: 1.

11. The recombinant expression vector of claim 1, comprising a nucleotide sequence at least 95% identical to SEQ ID NO: 1.

12. The recombinant expression vector of claim 1, comprising the nucleotide sequence of SEQ ID NO: 1.

13. The recombinant expression vector of claim 1, further comprising the nucleotide sequence of any one of SEQ ID NO: 2-7 inserted at the engineered MCS.

14. The recombinant expression vector of claim 1, further comprising a bicistronic gene element inserted at the engineered MCS, wherein the bicistronic gene element comprises (i) the nucleotide sequence of any one of SEQ ID NO: 2-7 and (ii) the nucleotide sequence of SEQ ID NO: 9.

15. The recombinant expression vector of claim 1, further comprising a bicistronic gene element inserted at the engineered MCS, wherein the bicistronic gene element comprises (i) the nucleotide sequence encoding a SARS-COV-2 spike protein amino acid sequence or a modified SARS-COV-2 spike protein amino acid sequence and (ii) a nucleotide sequence encoding a leader sequence.

16. The recombinant expression vector of claim 15, wherein the nucleotide sequence encoding the leader sequence comprises the nucleotide sequence of SEQ ID NO: 8 or 11.

17. The recombinant expression vector of claim 1, further comprising a bicistronic gene element inserted at the engineered MCS, wherein the bicistronic gene element comprises (i) the nucleotide sequence encoding a SARS-COV-2 spike protein amino acid sequence or a modified SARS-COV-2 spike protein amino acid sequence and (ii) a 3′ untranslated region (UTR).

18. The recombinant expression vector of claim 17, wherein the 3′ UTR comprises the nucleotide sequence of SEQ ID NO: 12 or SEQ ID NO: 13.

19. A pharmaceutical composition comprising the recombinant expression vector of claim 1 and a pharmaceutically acceptable carrier.

20. The pharmaceutical composition of claim 19, wherein the pharmaceutically acceptable carrier is lipid nanoparticles.

21. The pharmaceutical composition of claim 20, wherein the lipid nanoparticle comprises:
(a) an ionizable cationic lipid;
(b) a structural lipid;
(c) a stabilizer; and
(d) a sterol.

22. The pharmaceutical composition of claim 20, wherein the lipid nanoparticle comprises from about 20 mol % to about 70 mol % ionizable cationic lipid.

23. The pharmaceutical composition of claim 20, wherein the lipid nanoparticle comprises from about 5 mol % to about 45 mol % structural lipid.

24. The pharmaceutical composition of any claim 20, wherein the lipid nanoparticle comprises from about 15 mol % to about 45 mol % sterol.

25. The pharmaceutical composition of any claim 20, wherein the lipid nanoparticle comprises from about 0.2 mol % to about 5 mol % stabilizer.

26. The pharmaceutical composition of claim 20, wherein the nanoparticles have a size from about 50 nm to about 130 nm.
